(19) United States
(12) Patent Application Publication
(10) Pub. No.: US 2019/0000846 A1
(43) Pub. Date: Jan. 3, 2019

(54) Pharmaceutical Products and Drug Combinations for Treating Atherosclerosis by Stabilizing Atherosclerotic Plaques and Promoting Plaque Regression

(71) Applicants: Mayo Foundation for Medical Education and Research, Rochester, MN (US); Buck Institute for Research on Aging, Novato, CA (US); Unity Biotechnology, Inc., Brisbane, CA (US)

(72) Inventors: Jan M.A. van Deursen, Rochester, MN (US); Bennett G. Childs, Rochester, MN (US); Darren J. Baker, Rochester, MN (US); James L. Kirkland, Rochester, MN (US); Tamar Tchkonia, Rochester, MN (US); Yi Zhu, Rochester, MN (US); Nathaniel David, Brisbane, CA (US); Remi-Martin Laberge, San Francisco, CA (US); Judith Campisi, Berkeley, CA (US); Marco Demaria, Groningen (NL)

(21) Appl. No.: 16/025,238

(22) Filed: Jul. 2, 2018

Related U.S. Application Data
(63) Continuation-in-part of application No. 15/792,593, filed on Oct. 24, 2017.
Application No. 15/792,593 is a continuation-in-part of application No. 15/114,762, filed on Jul. 27, 2016, now Pat. No. 9,993,472.
(60) Provisional application No. 61/932,704, filed on Jan. 28, 2014.
(60) Provisional application No. 61/932,711, filed on Jan. 28, 2014.
(60) Provisional application No. 61/979,911, filed on Apr. 15, 2014.
(60) Provisional application No. 62/002,709, filed on May 23, 2014.
(60) Provisional application No. 62/042,708, filed on Aug. 27, 2014.
(60) Provisional application No. 62/044,664, filed on Sep. 2, 2014.
(60) Provisional application No. 62/057,820, filed on Sep. 30, 2014.
(60) Provisional application No. 62/057,825, filed on Sep. 30, 2014.
(60) Provisional application No. 62/057,828, filed on Sep. 30, 2014.
(60) Provisional application No. 62/061,627, filed on Oct. 8, 2014.
(60) Provisional application No. 62/061,629, filed on Oct. 8, 2014.
(60) Provisional application No. 62/412,223, filed on Oct. 24, 2016.
(60) Provisional application No. 62/412,605, filed on Oct. 25, 2016.

(51) Int. Cl. A61K 31/506; A61P 9/14; A61K 31/40; A61K 31/22

(52) U.S. Cl. CPC A61K 31/506; A61P 9/14; A61K 45/06; A61K 31/22; A61K 31/40

(57) ABSTRACT

Foamy macrophages with senescence markers accumulate in the subendothelial space at the onset of atherosclerosis where they drive pathology by increasing expression of key atherogenic and inflammatory cytokines and chemokines. This invention provides senolytic agents that remove senescent cells that are present in or around atherosclerotic plaques. The agents inhibit or reverse thinning of the fibrous cap on atherosclerotic plaques. This has the effect of stabilizing the plaques, inhibiting rupture and preventing pathological sequelae that manifest as coronary artery disease. Senolytic agents used in this way complement the action of statins and other drugs that cause plaque regression. Thus, senolytic agents and lipid lowering drugs can be used in combination for enhanced therapeutic effect.

### CROSS-REFERENCE TO RELATED APPLICATIONS

This application is a continuation-in-part of U.S. patent application Ser. No. 15/792,593 (pending), filed Oct. 24, 2017, which is a continuation-in-part of U.S. patent application Ser. No. 15/114,762, filed Jul. 27, 2016, now U.S. Pat. No. 9,993,472, which is the U.S. National Stage of PCT/US2015/013387, international filing date Jan. 28, 2015, which claims the priority benefit of provisional applications 61/932,704, filed Jan. 28, 2014; 61/932,711, filed Jan. 28, 2014; 61/979,911, filed Apr. 15, 2014; 62/002,709, filed May 23, 2014; 62/042,708, filed Aug. 27, 2014, 62/044,664, filed Sep. 2, 2014; 62/057,820, filed Sep. 30, 2014; 62/057,825, filed Sep. 30, 2014; 62/057,828, filed Sep. 30, 2014; 62/061,627, filed Oct. 8, 2014; and 62/061,629, filed Oct. 8, 2014. Priority application Ser. No. 15/792,593 also claims priority benefit of provisional applications 62/412,223, filed Oct. 24, 2016 and 62/412,605, filed Oct. 25, 2016.

The aforelisted applications are all hereby incorporated herein by reference in their entirety for all purposes, including but not limited to the preparation and use of senolytic agents to treat atherosclerosis.

### STATEMENT OF FEDERALLY SPONSORED RESEARCH

Certain aspects of this invention were made with government support under Grant No. AG009909. AG017242, AG41122 and AG046061 awarded by the National Institutes of Health. The government has certain rights in this invention.

### BACKGROUND

Atherosclerosis is often referred to as a hardening or furring of the arteries and is caused by the formation of multiple atheromatous plaques within the arteries. Atherosclerosis (also called arteriosclerotic vascular disease or ASVD herein and in the art) is a form of arteriosclerosis in which an artery wall thickens. Symptoms develop when growth or rupture of the plaque reduces or obstructs blood flow; and the symptoms may vary depending on which artery is affected. Atherosclerotic plaques may be stable or unstable. Stable plaques regress, remain static, or grow slowly, sometimes over several decades, until they may cause stenosis or occlusion.

Unstable plaques are vulnerable to spontaneous erosion, fissure, or rupture, causing acute thrombosis, occlusion, and infarction long before they cause hemodynamically significant stenosis. Most clinical events result from unstable plaques, which do not appear severe on angiography; thus, plaque stabilization may be a way to reduce morbidity and mortality. Plaque rupture or erosion can lead to major cardiovascular events such as acute coronary syndrome and stroke (see, e.g., Du et al., BMC Cardiovascular Disorders 14:83 (2014); Grimm et al., Journal of Cardiovascular Magnetic Resonance 14:80 (2012)). Disrupted plaques were found to have a greater content of lipid, macrophages, and had a thinner fibrous cap than intact plaques (see, e.g., Felton et al., Arteriosclerosis, Thrombosis, and Vascular Biology 17:1337-45 (1997)).

The methods and materials provided in this disclosure constitute a new and improved approach to the management and care of subjects having this dangerous condition.

### INCORPORATION BY REFERENCE

Aspects of this work were disclosed previously in U.S. patent application Ser. No. 15/792,593, published as US 2018/0104222 A1, of which this is a continuation in part, and in U.S. patent application Ser. No. 15/114,762, published as US 2016/0339019 A1, of which the Ser. No. 15/792,593 application is a continuation-in-part. A related academic publication by B. G. Childs et al. appeared in Science 354(6311):472-477, 2016. These and all other publications, patents, and patent applications mentioned in this specification are herein incorporated by reference in their entirety for all purposes, to the same extent as if each individual publication, patent, or patent application was specifically and individually indicated to be incorporated by reference.

### BRIEF DESCRIPTION OF THE DRAWINGS

FIG. 1A-1D: p16^{Ink4a}-positive senescent cells drive formation of atherosclerotic plaques. The left of FIG. 1A shows examples of three types of senescent cells observed by Gal-EM in plaques of Ldlr^{-/-} mice on a HFD for 88 days. Cell outlines are traced in dashed green. Endothelial-like cells are elongated and adjacent to the lumen. VSMC-like cells are elongated spindle shaped cells or irregularly shaped cells ramified cells. Macrophage-like cells are highly vacuolated, circular cells. On the right FIG. 1A shows senescent cell quantification in plaques with and without clearance. FIG. 1B illustrates experimental design for testing the effect of senescent cell clearance on atherogenesis (left), and Sudan IV-stained descending aortas (not including the arch)(right). FIG. 1C illustrates quantification of total descending aorta plaque burden, number, and lesion size. FIG. 1D illustrates analysis by qRT-PCR of senescence markers in aortic arches of indicated genotypes and treatments. Treatment in FIG. 1B and FIG. 1D: 5 mg/kg GCV or PBS daily for 5 days followed by 14 days off on a repeating cycle for 88 days. Scale bars: 2 μm (FIG. 1A) and 500 nm (FIG. 1A, insets). Bar graphs represent mean±SEM. *P<0.05; **P<0.01; ***, P<0.001 (unpaired two-tailed t-tests with Welch's correction).

FIG. 2A-2F: Intimal senescent foamy macrophages form during early atherogenesis and foster production of proatherogenic factors. FIG. 2A, Schematic of inner curvature (left) with examples of SA-β-Gal stained 9-day fatty streaks with and without senescent cell clearance and quantification (middle) and measurements of streak size (right). Treatment, 25 mg/kg GCV 1× daily. FIG. 2B, TEM images of Ldlr^{-/-} mice after 9-day HFD feeding showing fatty streak foci with X-Gal-positive foam cell macrophages (artificial coloring articulates cell boundaries in the multilayer). FIG. 2C, Quantification of multilayer foci in day-9 fatty streaks with and without senescent cells. FIG. 2D, Quantification of foam cell macrophages with X-Gal crystal-containing vesicles without and with clearance. FIG. 2E. Left, representative SA-β-Gal stained 12-day fatty streaks without and with GCV treatment for the last 3 days (25 mg/kg GCV 3× daily). Right, quantification of lesion burden. FIG. 2F, qRT-PCR analysis of senescence marker expression in fatty streaks collected from Ldlr^{-/-} and Ldlr^{-/-};3MR mice on a 12-day HFD and treated with GCV for the last 3 days. Scale bars: 1 mm (FIG. 2A, FIG. 2E); 2 μm (FIG. 2B) and 500 nm (FIG. 2B, insets) Bar graphs represent mean±SEM. *P<0.05; **P<0.01; ***P<0.001 (unpaired two-tailed t-tests with Welch's correction).

FIG. 3A-3D: Removal of p16^{Ink4a+} cells in established plaques perturbs the proatherogenic microenvironment. FIG. 3A, Left, experimental design for testing the effect of senescent cell clearance on established plaques. Middle, Sudan IV-stained descending aortas (not including the arch). Right, quantification of Sudan IV⁺ areas and plaque number. FIG. 3B, Left, experimental design for LFD switching. Middle, Sudan IV-stained descending aortas (not including the arch). Right, quantification of Sudan IV⁺ and abnormal intimal areas. FIG. 3C, SA-β-Gal staining of whole aortas (experimental design as in FIG. 3B). Insets correlate to color-matched boxes on low-power view. FIG. 3D, qRT-PCR for senescence markers in aortic arches from indicated cohorts. Aortic arches from Ldlr^{-/-};3MR females fed LFD until 258 days of age and treated with Veh for the last 100 days were used to assess baseline expression levels. Treatments in FIGS. 3A-D, 5 mg/kg GCV (or Veh) daily for 5 days followed by 14 days off on a repeating cycle for 100 days. Bar graphs represent mean±SEM. *P<0.05; **P<0.01; ***P<0.001 (FIG. 3A and FIG. 3B, ANOVA with Sidak's post-hoc correction for familywise error; FIG. 3D, unpaired two-tailed t-test with Welch's correction).

FIG. 4A-4C: Senescent cells promote plaque instability by elevating metalloprotease production. FIG. 4A, Representative sections from descending aorta plaques of mice with the indicated genotypes, treatments, diets and histological stainings. Red dashed lines trace the fibrous cap and red arrowheads indicated ruptured aortic elastic fibers. FIG. 4B, Quantification of fibrous cap thickness in plaques from FIG. 4A. FIG. 4C: top, experimental overview, bottom, qRT-PCR analysis of senescence markers in GFP⁺ and GFP⁻ cells. Bar graphs represent mean±SEM. *P<0.05; **P<0.01; ***P<0.001 (FIG. 4B, ANOVA with Sidak's post-hoc correction for familywise error; FIG. 4C, unpaired two-tailed 1-test with Welch's correction).

FIG. 5A-5C: Senescent cells accumulate in atherosclerotic plaques and are cleared by p16-3MR. FIG. 5A, Scheme of plaque induction protocol as well as schematic of heart and aorta alongside SA-β-Gal-stained aortas. LV, left ventricle; BCA, brachiocephalic artery. Inset corresponds to boxed region on low-power view. FIG. 5B, Senescence marker expression in aortic arches from Ldlr^{-/-} mice fed a HFD for 88 days versus LFD-fed controls. C, SA-β-Gal-stained descending aorta plaques from Ldlr^{-/-} and Ldlr^{-/-};3MR mice fed HFD for 88 days, followed by 5 weeks of LFD and GCV treatment. Scale bars: 100 pun (FIG. 5A, inset) and 500 μm (FIG. 5C) Bar graphs represent mean±SEM. *, P<0.05; **P<0.01; ***P<0.001 (unpaired t-test with Welch's correction).

FIG. 6A-6E: Senescent cell clearance is athero-protective in the brachiocephalic artery. FIG. 6A, Representative sections of plaque in brachiocephalic arteries. Plaque is traced with red dashed lines which, for clarity, extend through the vascular wall although plaque is only measured above the most superficial elastic fiber. Arrowheads in insets indicate broken elastic fibers. FIG. 6B, Quantification of average cross-sectional area of plaque in brachiocephalic arteries. FIG. 6C. Quantification of fragmented aortic elastic fibers from brachiocephalic plaques in FIG. 6A. FIG. 6D, SA-β-Gal staining of whole aortas undergoing senescent cell clearance compared to controls. Scale bars, 100 μm (FIG. 6A) and 20 μm (FIG. 6A, insets). Bar graphs represent mean±SEM. *, P<0.05; **P<0.01; ***P<0.001 (unpaired t-test with Welch's correction).

FIG. 7A-7I: Parameters that modulate atherosclerosis are not impacted by 16^{Ink4a}+ cell killing or GCV. FIG. 7A, Total body weight of mice with indicated diets, treatments, and genotypes enrolled in constitutive p16^{Ink4a+} cell clearance study. FIG. 7B, Body fat percentage measured by MRI. FIG. 7C. Mesentric fat mass measurements. FIG. 7D, Inguinal white adipose tissue (iWAT) mass measurements. FIGS. 7E-H. Circulating cell counts for eosinophils (FIG. 7E), lymphocytes (FIG. 7F), monocytes (FIG. 7G), and platelets (FIG. 7H) as measured by Hemavet blood analyzer. FIG. 7I, Plasma lipid profile. Bar graphs represent mean±SEM. *P<0.05; **P<0.01; ***P<0.001 (ANOVA with Sidak's post-hoc correction for familywise error).

FIG. 8A-8B: INK_ATTAC-mediated senescent cell killing blunts atherogenesis. A, sudan IV-stained descending aortas of the indicated mice. Experimental design: Ldlr^{-/-} female with or without the INK-ATTAC transgene were fed a LFD between 21 and 70 days of age and then switched to a HFD until aortas were dissected and analyzed at 172 days of age. AP20187 (AP) treatment (twice weekly) was started when animals were switched to a HFD. FIG. 8B, Quantification of Sudan IV⁺ area, plaque number, and individual plaque size in mice from FIG. 8A. Bar graphs represent mean±SEM. *P<0.05 (unpaired two-tailed t-tests with Welch's correction).

FIG. 9A-9C: Senescent cell killing by INK-NTR attenuates plaque initiation and growth. FIG. 9A, Schematic of the INK-NTR transgene. FIG. 9B, Experimental design and Sudan IV-stained whole aortas. Ldlr^{-/-} females with or without the INK-NTR transgene were fed a LFD between 21 and 70 days of age and then switched to a HFD and given ad libitum access to drinking water containing metronidazole until aortas were dissected and analyzed at 158 days of age. FIG. 9C, Quantification of total Sudan IV⁺ area, plaque number, and individual plaque size in mice from FIG. 9B. Bar graphs represent mean±SEM. *P<0.05 (unpaired two-tailed t-tests with Welch's correction).

FIG. 10A-10B: ABT263-mediated senescent cell clearance inhibits atherogenesis. FIG. 10A, Experimental design and representative Sudan-IV-stained descending aortas from Ldlr^{-/-} mice fed a HFD for 88 days and treated with either vehicle or ABT263. FIG. 10B, Total plaque burden, plaque number, and individual plaque size in mice from FIG. 10A. Bar graphs represent mean±SEM. *, P<0.05; ***P<0.001 (unpaired t-test with Welch's correction).

FIG. 11A-11D: Transgenic and pharmacological elimination of senescent cells inhibits fatty streak formation. FIG. 11A, qRT-PCR analysis of senescence markers, monocyte chemotactic factors, cytokines, and proteases in the inner curvature of the aortic arch of Ldlr^{-/-} mice fed LFD or HFD for 9 days. FIG. 11B, Left. SA-β-Gal stained fatty streaks from Ldlr^{-/-} mice fed a HFD for 9 days with either vehicle or ABT263 treatment. Right, quantification of fatty streak burden in the aortic arch. FIG. 11C, Quantification of percentage of X-Gal-positive foam cell macrophages in inner curvatures from Ldlr^{-/-} or Ldlr^{-/-};3MR mice with 9-day fatty streaks treated with GCV for 3 days. FIG. 1D, Representative TEM images of plaques from Ldlr^{-/-} or Ldlr^{-/-};3MR mice with 9-day fatty streaks treated with GCV for 3 days. Basement membrane is traced in dashed white line for clarity, and subendothelial contents are false-colored in red. In the Ldlr^{-/-} panel, individual macrophage foam cells are different shades of red. In the Ldlr^{-/-};3MR, insets demonstrate the diffuse acellular debris retained in the subendothelium following three days of senescent cell killing. Scale bars: 1 mm (FIG. 11B), 2 μm (FIG. 11D), 500 nm and (FIG. 11D, inset). Bar graphs represent mean±SEM. *, P<0.05; ***P<0.001 (unpaired t-test with Welch's correction).

FIG. 12A-12C: Senescent cell killing in advanced plaques reduces monocyte chemotactic factors without impacting plasma lipids. FIG. 12A, Lipid profile from mice fed a LFD or HFD for the indicated durations, genotypes, and drug treatments. FIG. 12B, qRT-PCR analysis of CD11b expression in aortic arches with advanced plaques from FIG. 12A. FIG. 12C, qRT-PCR analysis of monocyte chemotactic factors in aortic arches with advanced plaques, with and without senescent cell clearance. Bar graphs represent mean±SEM. *P<0.05; **P<0.01; ***P<0.001 (ANOVA with Sidak's post-hoc correction for familywise error).

FIG. 13A-13B: Senescent cells reduce collagen and elastin content in advanced plaques. FIG. 13A, Quantification of collagen (blue colored regions) in paraffin sections of advanced plaques stained with Masson's trichrome. FIG. 13B. Quantification of aortic elastic fibers underlying the plaque neointima with one or more interruptions.

FIG. 14A-14B: Senescent cell clearance increases fibrous cap thickness and collagen content in brachiocephalic artery plaques. FIG. 14A, Representative images of brachiocephalic artery plaques. A, Representative images of brachiocephalic artery plaques from Ldlr^{-/-} or Ldlr^{-/-};3MR mice given the indicated diets or treatments and stained with Masson's trichrome (top) or H-E (bottom). FIG. 14B, Quantification of fibrous cap thickness from mice in FIG. 14A. FIG. 14C, Quantification of blue-stained collagen in advanced plaques from A visualized using Masson's trichrome. Bar graphs represent mean±SEM. *, P<0.05 (unpaired t-test with Welch's correction).

FIG. 15A-15G: Established plaques are stabilized by short-term senescent cell removal. FIG. 15A, Schematic showing the design of the short-term senescent cell killing experiment. FIG. 15B, Examples of fibrous caps (indicated between red dashed lines) from plaques undergoing high-intensity clearance. FIG. 15C-E, Quantification of fibrous cap thickness (FIG. 15C), VSMC-like cell density (FIG. 15D), and macrophage-like cell density (FIG. 15E) in plaques. FIG. 15F, Calculation of the VSMC- to macrophage-like cell ratio in plaques. FIG. 15G, Representative image of a monocyte bound to endothelium adjacent to a plaque. FIG. 15H, Quantification of adherent monocytes in plaque sections from FIG. 15B. Scale bar, 10 μm (FIG. 15B) and 2 μm (FIG. 15G). Bar graphs represent mean±SEM. *P<0.05 (unpaired two-tailed t-tests with Welch's correction).

FIG. 16: Pharmacological senolysis blocks fibrous cap thinning in aortic arch lesions. Ldlr^{-/-} mice were fed a high-fat diet (HFD) for 3 months to develop mature, thick-cap fibroatheromas in the aortic arch and brachiocephalic artery, and then switched to a low-fat diet (LFD) to imitate lipid-normalizing statin treatment. ABT263 (navitoclax) or vehicle was administered once-per day intraperitoneally at a dose of 100 mg/kg for 9 weeks (3 cycles consisting of 7 days of treatment followed by 14 days of rest. In vehicle treated mice, the aortic arch fibrous cap thins by ˜25% during the 9-week LFD feeding interval despite plaque cross-sectional area remaining the same. Treatment with ABT263 completely blocks fibrous cap thinning.

FIG. 17: Pharmacological senolysis blocks fibrous cap thinning in brachiocephalic artery lesions.

FIG. 18: Pharmacological senolysis does not block diet-induced regression of plaque. Treatment with ABT263 during the 9-week feeding interval completely blocked fibrous cap thinning and, importantly, did not disrupt plaque debulking that occurred as a result of lipid normalization.

FIG. 19: Pharmacological senolysis thickens maximally thinned fibrous caps. If ABT263 is administered during the 9 weeks of LFD feeding, the fibrous cap increases in thickness by ˜29%. This result indicates that pharmacological senolysis is not only capable of blocking fibrous cap thinning, but can, in extremely thin-capped fibroatheromas, actually thicken a thin fibrous cap.

### DETAILED DESCRIPTION OF THE INVENTION

Foamy macrophages with senescence markers accumulate in the subendothelial space at the onset of atherosclerosis where they drive pathology by increasing expression of key atherogenic and inflammatory cytokines and chemokines. This invention provides senolytic agents that remove senescent cells that are present in or around atherosclerotic plaques. The agents inhibit or reverse thinning of the fibrous cap on atherosclerotic plaques. This has the effect of stabilizing the plaques, inhibiting rupture and preventing pathological sequelae that manifest as coronary artery disease. Senolytic agents used in this way complement the action of statins and other drugs that cause plaque regression. Thus, senolytic agents and lipid lowering drugs can be used in combination for enhanced therapeutic effect.

### Features of Atherosclerotic Plaques

Atherosclerosis is characterized by patchy intimal plaques (atheromas) that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and its branches, and major arteries of the extremities. Atherosclerosis is characterized by patchy intimal plaques (atheromas) that encroach on the lumen of medium-sized and large arteries; the plaques contain lipids, inflammatory cells, smooth muscle cells, and connective tissue.

Atherosclerosis is a syndrome affecting arterial blood vessels due in significant part to a chronic inflammatory response of white blood cells in the walls of arteries. This is promoted by low-density lipoproteins (LDL, plasma proteins that carry cholesterol and triglycerides) in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic plaque, which is an evolution of the fatty streak and has three major components: lipids (e.g., cholesterol and triglycerides); inflammatory cells and smooth muscle cells; and a connective tissue matrix that may contain thrombi in various stages of organization and calcium deposits.

Within the outer-most and oldest plaque, calcium and other crystallized components (e.g., microcalcification) from dead cells can be found. Microcalcification and properties related thereto are also thought to contribute to plaque instability by increasing plaque stress (see, e.g., Bluestein et al., J. Biomech. 41(5): 1111-18 (2008); Cilla et al., Journal of Engineering in Medicine 227:588-99 (2013)). Fatty streaks reduce the elasticity of the artery walls, but may not affect blood flow for years because the artery muscular wall accommodates by enlarging at the locations of plaque. Lipid-rich atheromas are at increased risk for plaque rupture and thrombosis (see, e.g., Felton et al., supra; Fuster et al., J. Am. Coll. Cardiol. 46:1209-18 (2005)). Reports have found that of all plaque components, the lipid core exhibits the highest thrombogenic activity (see, e.g., Fernandez-Ortiz et al., J. Am. Coll. Cardiol. 23:1562-69 (1994)). Within major arteries in advanced disease, the wall stiffening may also eventually increase pulse pressure.

A vulnerable plaque that may lead to a thrombotic event (stroke or MI) and is sometimes described as a large, soft lipid pool covered by a thin fibrous cap (see, e.g., Li et al., Stroke 37:1195-99 (2006); Trivedi et al., Neuroradiology 46:738-43 (2004)). An advanced characteristic feature of advance atherosclerotic plaque is irregular thickening of the arterial intima by inflammatory cells, extracellular lipid (atheroma) and fibrous tissue (sclerosis) (see, e.g., Newby et al., Cardiovasc. Res. 345-60 (1999)). Fibrous cap formation is believe to occur from the migration and proliferation of vascular smooth muscle cells and from matrix deposition (see, e.g., Ross, Nature 362:801-809 (1993); Sullivan et al., J. Angiology at dx.doi.org/10.1155/2013/592815 (2013)). A thin fibrous cap contributes instability of the plaque and to increased risk for rupture (see, e.g., Li et al., supra).

Both proinflammatory macrophages (M1) and anti-inflammatory macrophages (M2) can be found in arteriosclerotic plaque. The contribution of both types to plaque instability is a subject of active investigation, with results suggesting that an increased level of the M1 type versus the M2 type correlates with increased instability of plaque (see, e.g., Medbury et al., Int. Angiol. 32:74-84 (2013); Lee et al., Am. J. Clin. Pathol. 139:317-22 (2013); Martinet et al., Cir. Res. 751-53 (2007)).

Generally, diagnosis of atherosclerosis and other cardiovascular disease is based on symptoms (e.g., chest pain or pressure (angina), numbness or weakness in arms or legs, difficulty speaking or slurred speech, drooping muscles in face, leg pain, high blood pressure, kidney failure and/or erectile dysfunction), medical history, and/or physical examination of a patient. Diagnosis may be confirmed by angiography, ultrasonography, or other imaging tests. Subjects at risk of developing cardiovascular disease include those having any one or more of predisposing factors, such as a family history of cardiovascular disease and those having other risk factors (i.e., predisposing factors) such as high blood pressure, dyslipidemia, high cholesterol, diabetes, obesity and cigarette smoking, sedentary lifestyle, and hypertension. In a certain embodiment, the cardiovascular disease that is a senescence cell associated disease/disorder is atherosclerosis.

### Some Aspects of the Invention

The methods of the invention include administering to a subject in need thereof a therapeutically-effective amount of a small molecule senolytic agent that selectively kills senescent cells over non-senescent cells; wherein the senescence-associated disease or disorder is not a cancer, wherein the senolytic agent is administered in at least two treatment cycles, wherein each treatment cycle independently comprises a treatment course of from 1 day to 3 months followed by a non-treatment interval of at least 2 weeks; provided that if the senolytic agent is an MDM2 inhibitor, the MDM2 inhibitor is administered as a monotherapy, and each treatment course is at least 5 days long during which the MDM2 inhibitor is administered on at least 5 days. In certain embodiments, the senolytic agent is selected from an MDM2 inhibitor; an inhibitor of one or more Bcl-2 anti-apoptotic protein family members wherein the inhibitor inhibits at least Bcl-xL; and an Akt specific inhibitor. In a specific embodiment, the MDM2 inhibitor is a cis-imidazoline compound, a spiro-oxindole compound, or a benzodiazepine compound. In a specific embodiment, the cis-imidazoline compound is a nutlin compound. In a specific embodiment, the senolytic agent is an MDM2 inhibitor and is Nutlin-3a or RG-1172. In a specific embodiment, the nutlin compound is Nutlin-3a.

In a specific embodiment, the cis-imidazoline compound is RG-7112, RG7388, RO5503781, or is a dihydroimidazothiazole compound. In a specific embodiment, the dihydroimidazothiazole compound is DS-3032b. In a specific embodiment, the MDM2 inhibitor is a spiro-oxindole compound selected from MI-63, MI-126, MI-122, MI-142, MI-147, MI-18, MI-219, MI-220, MI-221, MI-773, and 3-(4-chlorophenyl)-3-((1-(hydroxymethyl)cyclopropyl)methoxy)-2-(4-nitrobenzyl)isoindolin-1-one. In a specific embodiment, the MDM2 inhibitor is Serdemetan; a piperidinone compound; CGM097; or an MDM2 inhibitor that also inhibits MDMX and which is selected from RO-2443 and RO-5963.

In a specific embodiment, the piperidinone compound is AM-8553. In a specific embodiment, the inhibitor of one or more Bcl-2 anti-apoptotic protein family members is a Bcl-2/Bcl-xL inhibitor; a Bcl-2/Bcl-xL/Bcl-w inhibitor; or a Bcl-xL selective inhibitor. In a specific embodiment, the senolytic agent is an inhibitor of one or more Bcl-2 anti-apoptotic protein family members wherein the inhibitor inhibits at least Bcl-xL and is selected from ABT-263, ABT-737, WEHI-539, and A-1155463. In a specific embodiment, the Bcl-xL selective inhibitor is a benzothiazole-hydrazone compound, an aminopyridine compound, a benzimidazole compound, a tetrahydroquinolin compound, or a phenoxyl compound. In a specific embodiment, the benzothiazole-hydrazone compound is a WEHI-539. In a specific embodiment, the inhibitor of the one or more Bcl-2 anti-apoptotic protein family members is A-1155463. ABT-263, or ABT-737. In a specific embodiment, the Akt inhibitor is MK-2206.

A pharmaceutical composition may be delivered to a subject in need thereof by any one of several routes known to a person skilled in the art. By way of non-limiting example, the composition may be delivered orally, intravenously, intraperitoneally, by infusion (e.g., a bolus infusion), subcutaneously, enteral, rectal, intranasal, by inhalation, buccal, sublingual, intramuscular, transdermal, intradermal, topically, intraocular, vaginal, rectal, or by intracranial injection, or any combination thereof. In certain particular embodiments, administration of a dose, as described above, is via intravenous, intraperitoneal, directly into the target tissue or organ, or subcutaneous route. In certain embodiments, a delivery method includes drug-coated or permeated stents for which the drug is the senolytic agent. Formulations suitable for such delivery methods are described in greater detail herein.

A senolytic agent (which may be combined with at least one pharmaceutically acceptable excipient to form a pharmaceutical composition) can be administered directly to the target tissue or organ comprising senescent cells that contribute to manifestation of the disease or disorder. Methods are provided herein for treating a cardiovascular disease or disorder associated with arteriosclerosis, such as atherosclerosis by administering directly into an artery. In another particular embodiment, a senolytic agent (which may be combined with at least one pharmaceutically acceptable excipient to form a pharmaceutical composition) for treating a senescent-associated pulmonary disease or disorder may be administered by inhalation, intranasally, by intubation, or intracheally, for example, to provide the senolytic agent more directly to the affected pulmonary tissue. By way of another non-limiting example, the senolytic agent (or pharmaceutical composition comprising the senolytic agent) may be delivered directly to the eye either by injection (e.g., intraocular or intravitreal) or by conjunctival application underneath an eyelid of a cream, ointment, gel, or eye drops. In more particular embodiments, the senolytic agent or pharmaceutical composition comprising the senolytic agent may be formulated as a timed release (also called sustained release, controlled release) composition or may be administered as a bolus infusion.

A pharmaceutical composition (e.g., for oral administration or for injection, infusion, subcutaneous delivery, intramuscular delivery, intraperitoneal delivery or other method) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an opthalmological condition or disease, a liquid pharmaceutical composition may be applied to the eye in the form of eye drops. A liquid pharmaceutical composition may be delivered orally.

In certain embodiments of a method described herein for treating a cardiovascular disease associated with or caused by arteriosclerosis, one or more senolytic agents may be delivered directly into a blood vessel (e.g., an artery) via a stent. In a particular embodiment, a stent is used for delivering a senolytic agent to an atherosclerotic blood vessel (an artery). A stent is typically a tubular metallic device, which has thin-metal screen-like scaffold, and which is inserted in a compressed form and then expanded at the target site. Stents are intended to provide long-term support for the expanded vessel. Several methods are described in the art for preparing drug-coated and drug-embedded stents. For example, a senolytic agent may be incorporated into polymeric layers applied to a stent. A single type of polymer may be used, and one or more layers of the senolytic agent permeated polymer may be applied to a bare metal stent to form the senolytic agent-coated stent. The senolytic agent may also be incorporated into pores in the metal stent itself, which may also be referred to herein as a senolytic agent-permeated stent or senolytic agent-embedded stent.

A senolytic agent may be formulated within liposomes and applied to a stent; in other particular embodiments, for example, when the senolytic agent is ABT-263, the ABT-263 is not formulated in liposome. Placement of stents in an atherosclerotic artery is performed by a person skilled in the medical art. A senolytic agent-coated or -embedded stent not only expands the affected blood vessel (e.g., an artery) but also may be effective for one or more of (1) reducing the amount of plaque, (2) inhibiting formation of plaque, and (3) increasing stability of plaque (e.g., by decreasing lipid content of the plaque; and/or causing an increase in fibrous cap thickness), particularly with respect to plaque proximal to the agent coated or agent embedded stent.

Kits with unit doses of one or more of the agents described herein, usually in oral or injectable doses, are provided. Such kits may include a container containing the unit dose, an informational package insert describing the use and attendant benefits of the drugs in treating the senescent cell associated disease, and optionally an appliance or device for delivery of the composition.

### Empirical Observations Showing the Role of Senescent Foam Cells in Formation of Atherosclerotic Plaques

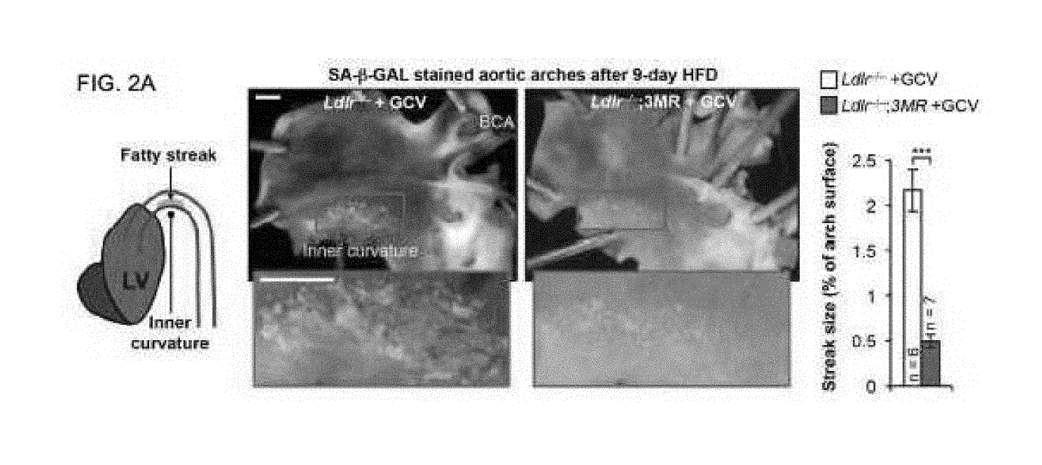

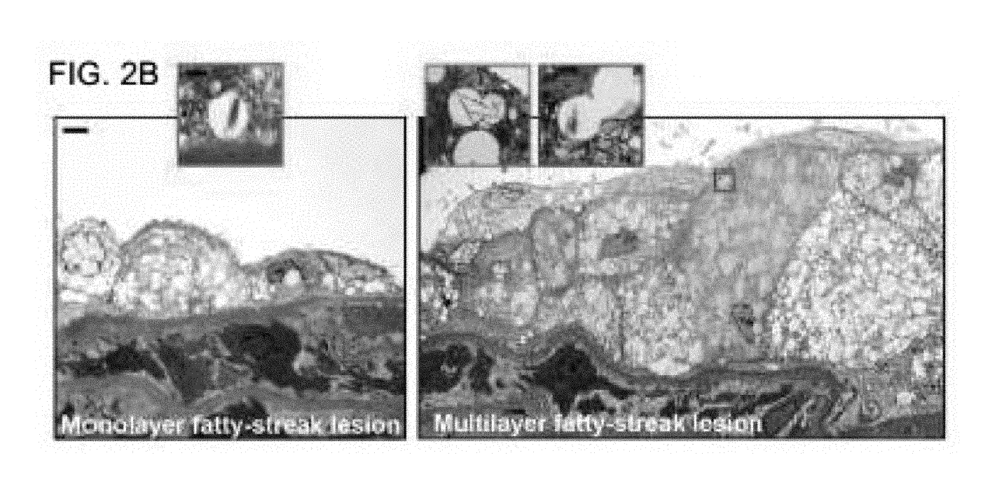

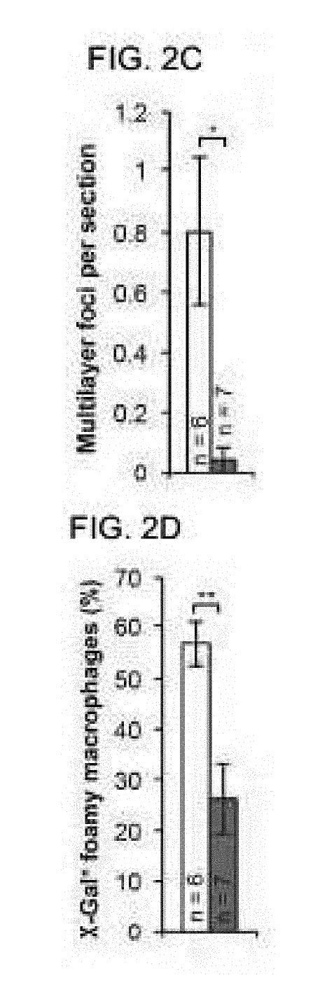

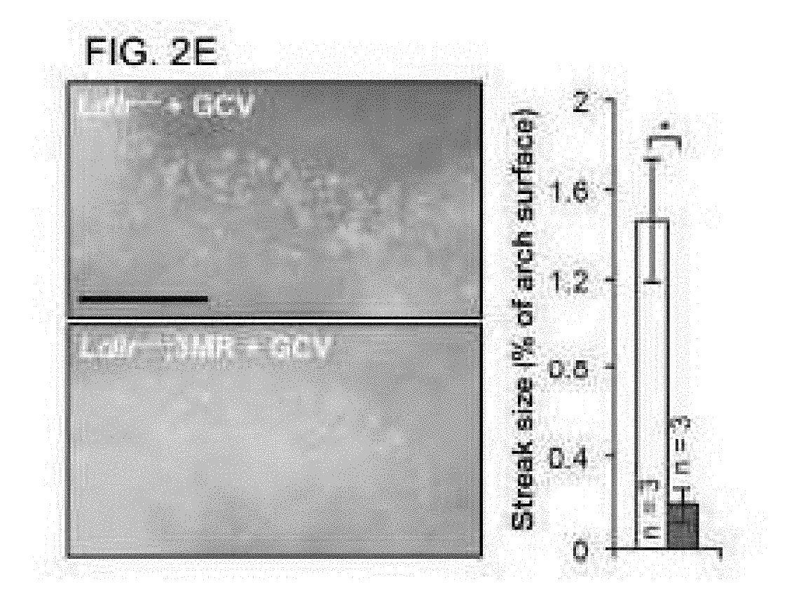

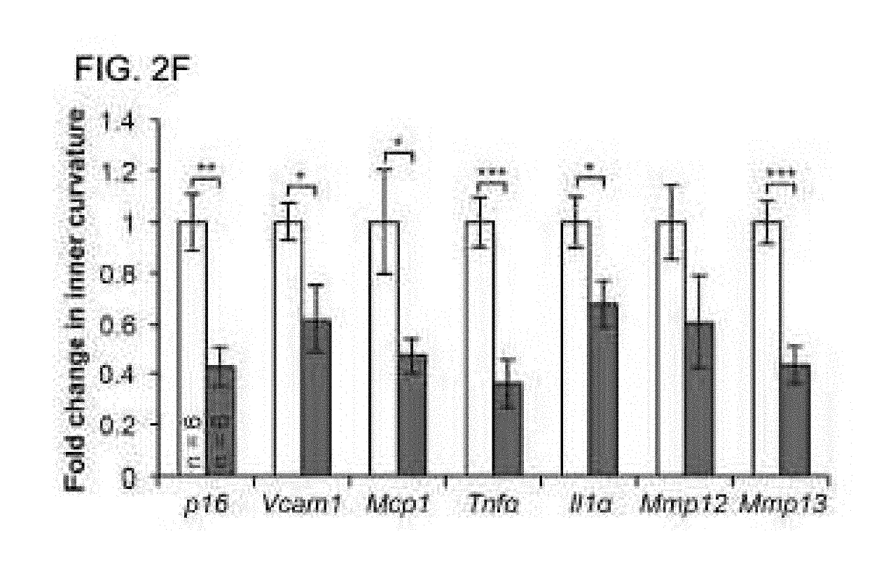

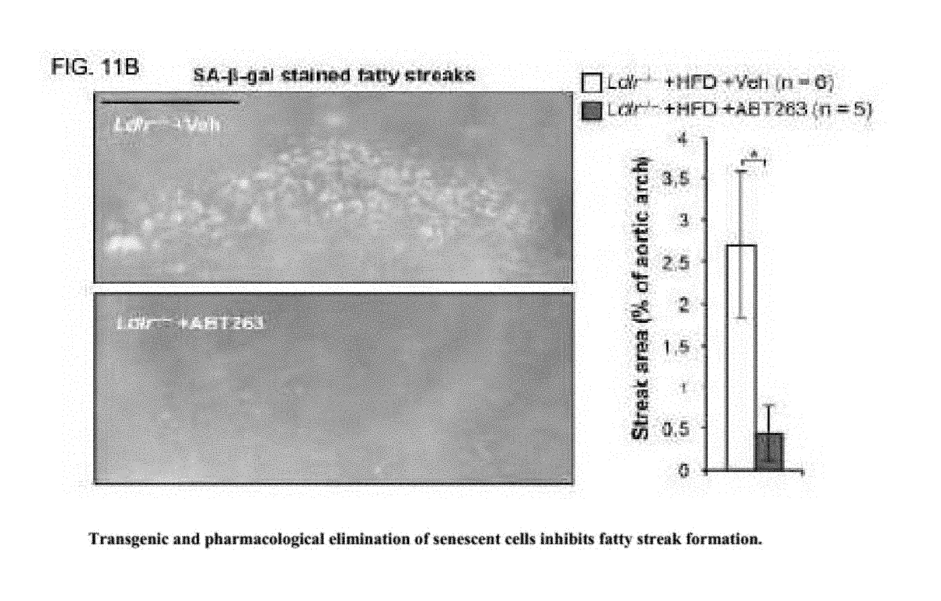

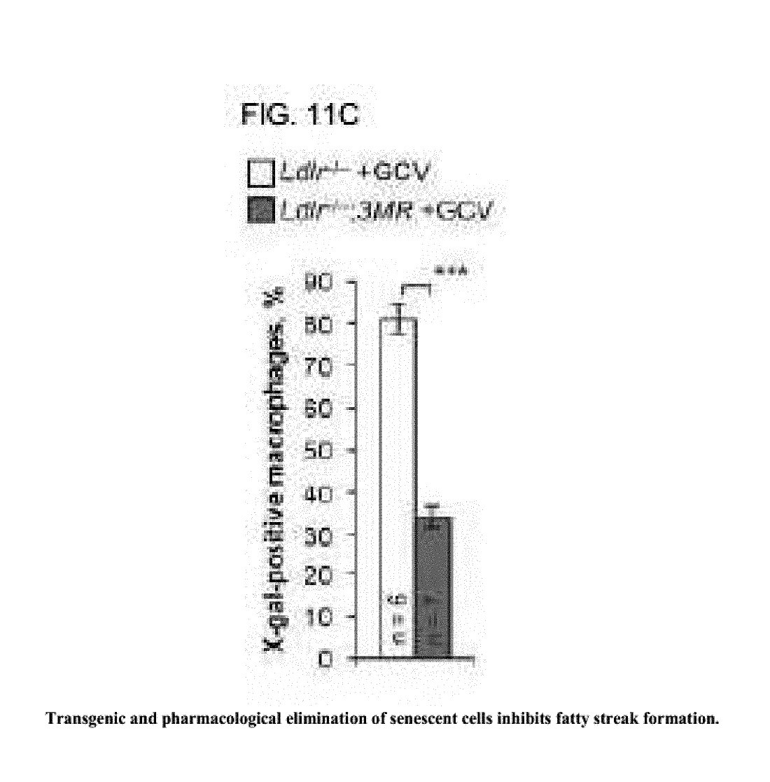

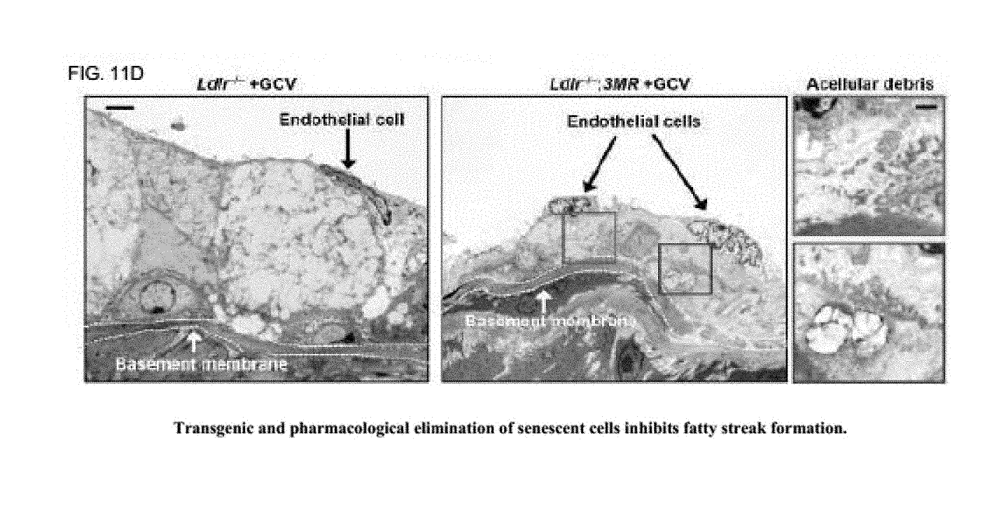

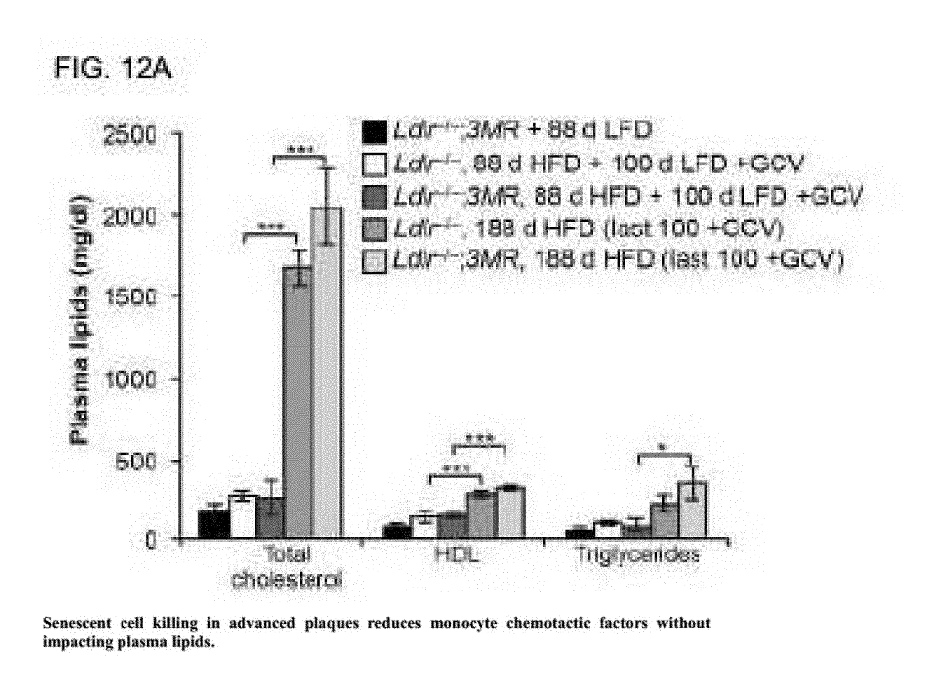

Using low-density lipoprotein knockout (LDLr−/−) mice on a high-fat diet as a model for human atherosclerosis, it is seen that that senescent foamy macrophages populate the subendothelial space within days after induction of hvpercholesterolemia (FIG. 2A and FIG. 2B). Clearance of senescent cells from early stage lesions using genetic (p16-3MR+ganciclovir FIGS. 2C-E) or pharmacological (ABT263; FIG. 11B) approaches, resulted in near complete lesion regression (FIG. 11D).

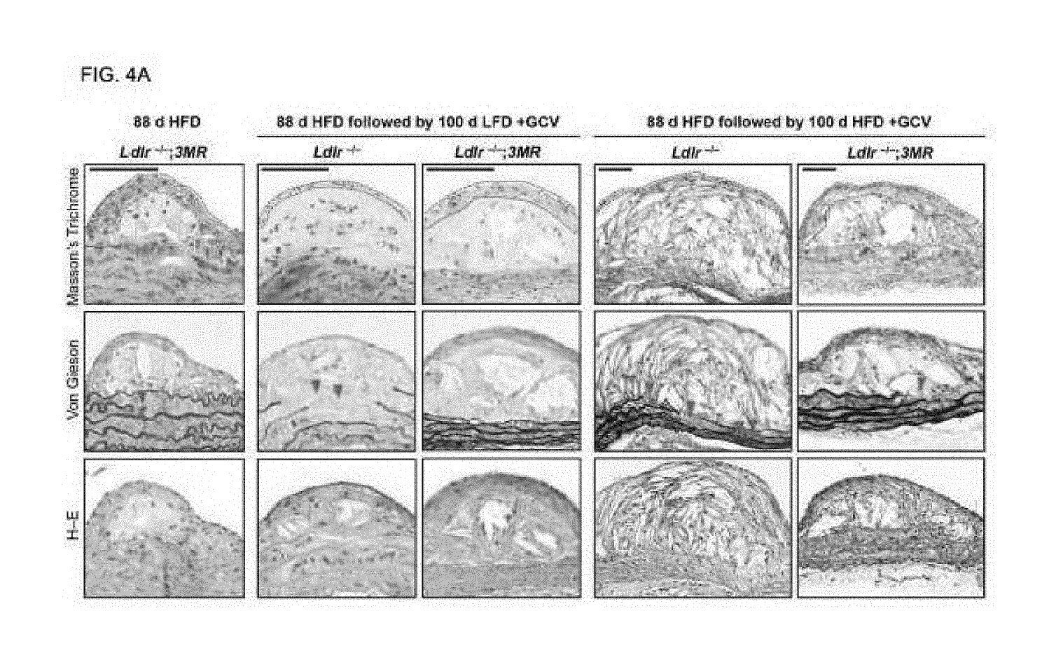

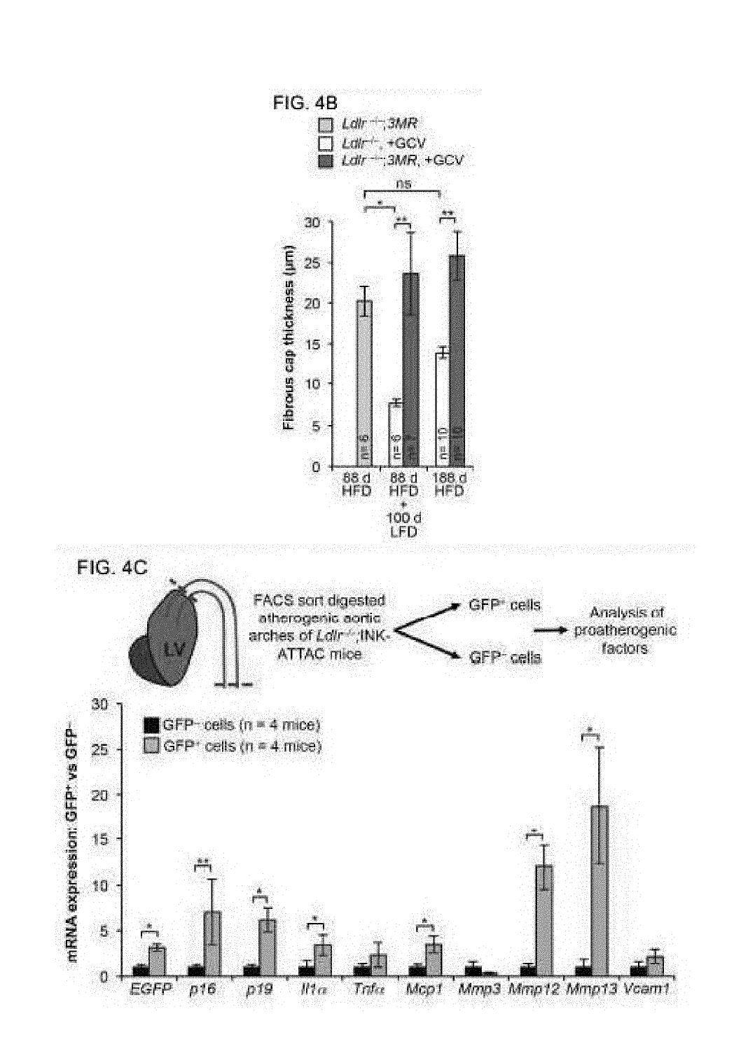

Mechanistically, senescent cells in early lesions are the main drivers of VMAC1 and MCP1 expression, two key monocyte recruitment factors that drive plaque growth by escalating foamy macrophage accumulation in the subendothelial space (FIG. 2F). Purification of senescent cells from atherogenic plaques reveals that senescent cells express both VCAM1 and MCP1, the latter in high abundance (FIG. 4C).

Thus, the elimination of senescent cells inhibits the growth of atherogenic lesions by blunting recruitment of circulating monocytes.

Additionally, senescent cells from mature plaques produce high levels of two matrix metalloproteinases. MMP12 and MMP13, that digest the fibrous cap that provides mature plaques with stability, thereby preventing plaque rupture, a major determining factor in the catastrophic consequences of atherosclerotic diseases, such as acute heart attacks and strokes (FIG. 4C). These findings explain why clearance of senescent cells from advanced plaques preserves plaque stability (fibrous cap thickness and elastic fiber integrity) (FIG. 4A, 4B).

p16^{Ink4a}-positive senescent foam cells accumulate throughout atherogenesis, where they are causally implicated in the formation of fatty streaks and their progression to large, vulnerable plaques by enhancing monocyte recruitment factors, inflammation, and matrix metalloprotease production. Atherosclerosis initiates when oxidized lipoprotein infiltrates the subendothelial space of arteries, often due to aberrantly elevated levels of apolipoprotein B-containing lipoproteins in the blood (1). Chemotactic signals arising from activated endothelium and vascular smooth muscle attract circulating monocytes that develop into lipid-loaded foamy macrophages, a subset of which adopt a proinflammatory phenotype through a mechanism that is not fully understood (2). The proinflammatory signals lead to additional rounds of monocyte recruitment and accumulation of other inflammatory cells including T and B cells, dendritic cells and mast cells, allowing initial lesions, often termed fatty streaks, to increase in size and develop into plaques (3). Plaque stability, rather than absolute size determines whether atherosclerosis is clinically silent or pathogenic because unstable plaques can rupture and produce vessel-occluding thrombosis and end-organ damage (4). Stable plaques have a relatively thick fibrous cap consisting largely of vascular smooth muscle cells (VSMCs) and extracellular matrix components, partitioning soluble clotting factors in the blood from thrombogenic molecules in the plaque (5). In advanced disease, plaques destabilize when elevated local matrix metalloprotease production degrades the fibrous cap, increasing the risk of lesion rupture and subsequent thrombosis.

Advanced plaques contain cells with markers of senescence, a stress response that entails a permanent growth arrest coupled to the robust secretion of numerous biologically active molecules, referred to as the senescence-associated secretory phenotype (SASP). The senescence markers include elevated senescence-associated β-galactosidase (SA-β Gal) activity and p16^{Ink4a}, p53 and p21 expression (6, 7). Human plaques contain cells with shortened telomeres, which predisposes cells to undergo senescence (10). Consistent with a proatherogenic role of senescence is the observation that expression of a loss-of-function telomere binding protein (Trf2) in VSMCs accelerates plaque growth in the ApoE^{-/-} mouse model of atherosclerosis, although evidence for increased in vivo senescence was not provided (10). On the other hand, mice lacking core components of senescence pathways, such as p53, p21 or p19^{Arf} (9, 11-13), show accelerated atherosclerosis, implying a protective role for senescence. Studies showing that human and mouse polymorphisms that reduce expression of p16^{Ink4a} and p14/19^{Arf} correlate with increased atheroma risk support this conclusion (9, 14, 15).

In the development of this invention, the role of naturally occurring senescent cells at different stages of atherogenesis was examined using genetic and pharmacological methods of eliminating such cells.

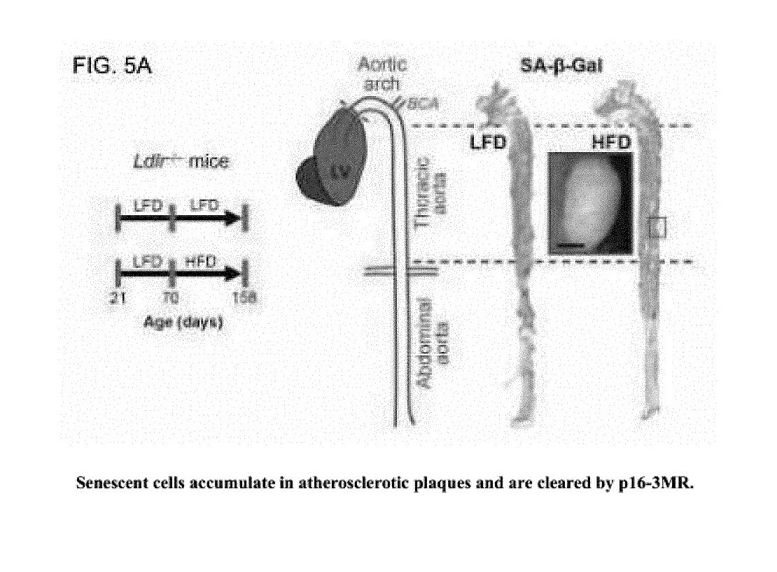

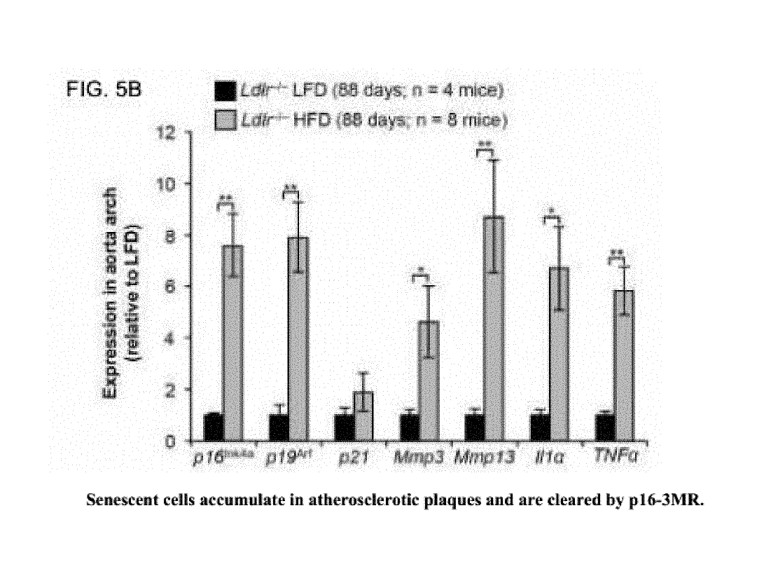

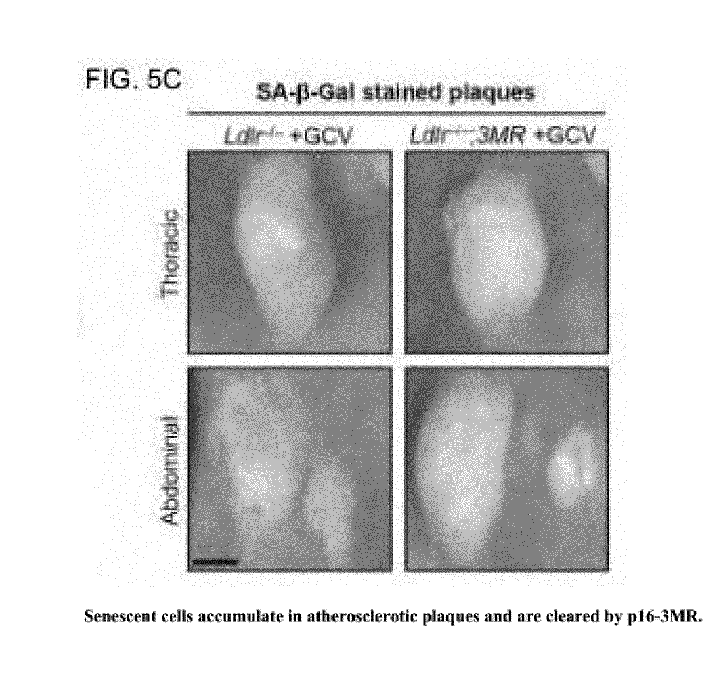

First, it was verified that senescent cells accumulate in LDL-receptor knockout (Ldlr^{-/-}) mice, a model of atherogenesis. To this end, 10-week-old Ldlr^{-/-} mice were fed a high-fat diet (HFD) for 88 days. Indeed, SA-β-Gal staining occurred in atherosclerotic lesions but not in the normal adjacent vasculature or aortas of low-fat diet (LFD)-fed Ldlr^{-/-} mice (FIG. 5A). In addition, plaque-rich aortic arches had elevated transcript levels of p16^{Ink4a}, p19^{Arf} and various canonical SASP components, including the matrix metalloproteases Mmp³ and Mmp¹³ and the inflammatory cytokines Il1α and Tnfα (FIG. 5B). To eliminate senescent cells from plaques p16-3MR mice (16) were used, a transgenic model that expresses the herpes simplex virus thymidine kinase (HSV-TK) under the control of the Cdkn2a promoter and kills p16^{Ink4a}-positive senescent cells upon administration of ganciclovir (GCV).

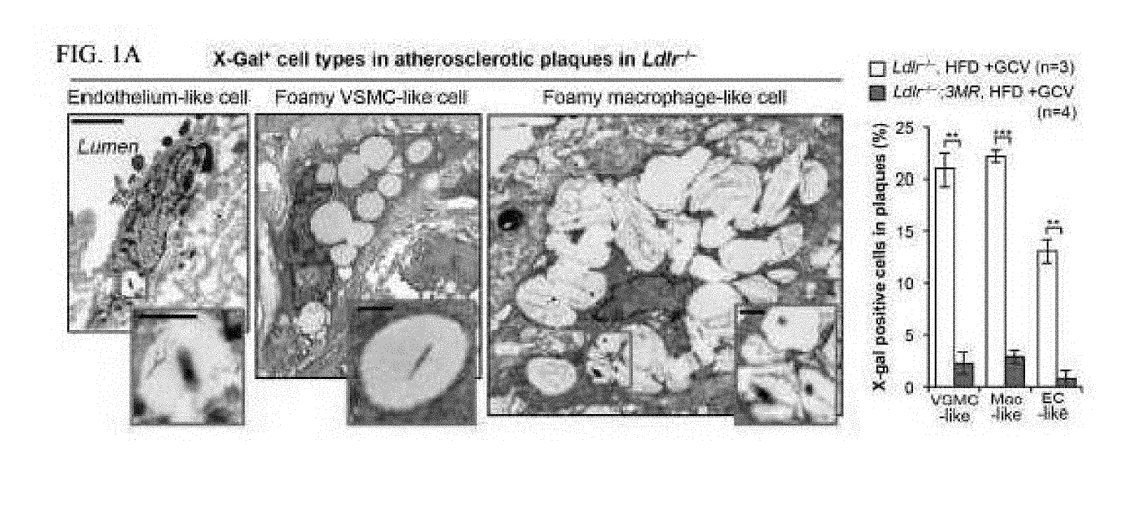

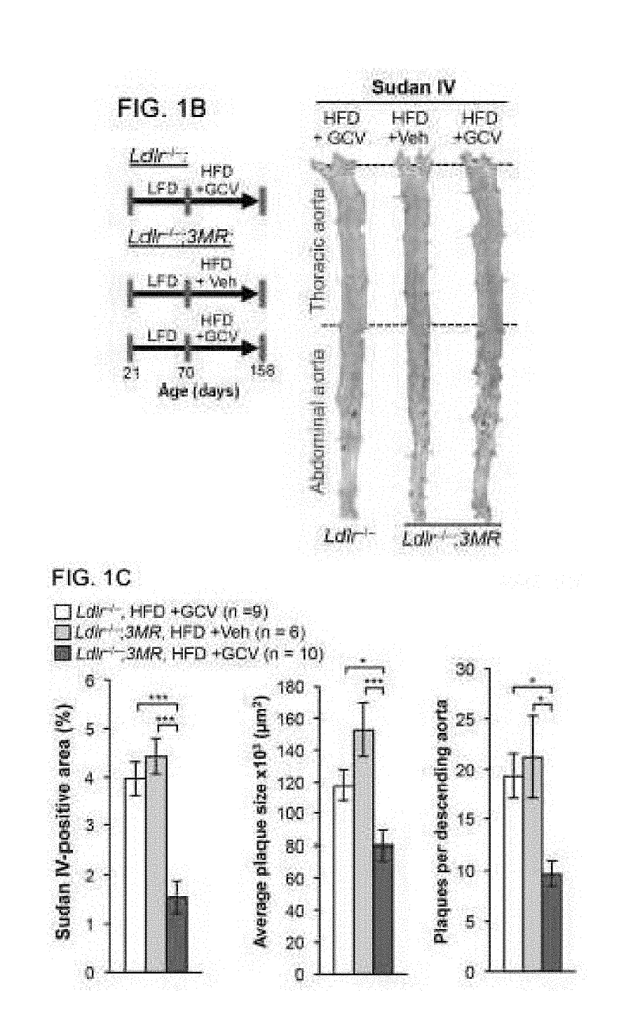

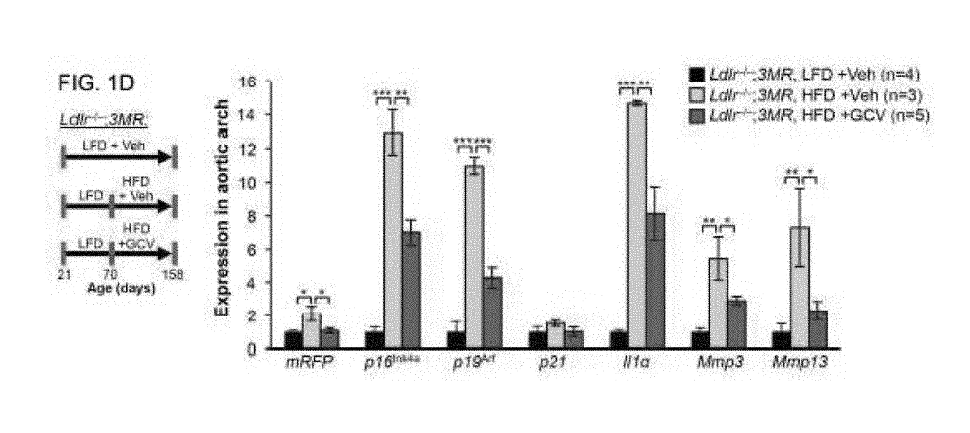

Plaques of Ldlr^{-/-};3MR mice fed a HFD for 88 days and then treated short term with GCV had low SA-β-Gal activity compared to those of Ldlr^{-/-} mice (FIG. 5C), indicating efficient clearance of senescent cells. Examination of the plaques by transmission electron microscopy (TEM) revealed that three morphologically distinct cell types produced X-Gal crystals: elongated, vacuolated cells located in the endothelial layer, spindly foam cells with histological properties of VSMCs, and large foamy cells resembling lipid-loaded macrophages (FIG. 1A). These cells were referred to as endothelial-, foamy VSMC- and foamy macrophage-like cells, respectively, because cells polymorphisms that reduce expression of p16^{Ink4a} and p14/19^{Arf} correlate with increased atheroma risk support this conclusion (9, 14, 15).

The role of naturally occurring senescent cells at different stages of atherogenesis was examined using genetic and pharmacological methods of eliminating senescent cells. First, it was verified that senescent cells accumulate in Ldl-receptor knockout (Ldlr^{-/-}) mice, a model of atherogenesis. To this end, 10-week-old Ldlr^{-/-} mice were fed a high-fat diet (HFD) for 88 days. Indeed, SA-β-Gal staining occurred in atherosclerotic lesions but not in the normal adjacent vasculature or aortas of low-fat diet (LFD)-fed Ldlr^{-/-} mice (FIG. 5A). In addition, plaque-rich aortic arches had elevated transcript levels of p16^{Ink4a}, p19^{Arf} and various canonical SASP components, including the matrix metalloproteases Mmp3 and Mmp13 and the inflammatory cytokines Il1α and Tnfα (FIG. 5B).

To eliminate senescent cells from plaques p16-3MR mice (16) were used, which are a transgenic model that expresses the herpes simplex virus thymidine kinase (HSV-TK) under the control of the Cdkn2a promoter and kills p16I^{nk4a}-positive senescent cells upon administration of ganciclovir (GCV). Plaques of Ldlr^{-/-};3MR mice fed a HFD for 88 days and then treated short term with GCV had low SA-j-Gal activity compared to those of Ldlr^{-/-} mice (FIG. 5C), indicating efficient clearance of senescent cells. Examination of the plaques by transmission electron microscopy (TEM) revealed that three morphologically distinct cell types produced X-Gal crystals: elongated, vacuolated cells located in the endothelial layer, spindly foam cells with histological properties of VSMCs, and large foamy cells resembling lipid-loaded macrophages (FIG. 1A). These cells were referred to as endothelial-, foamy VSMC- and foamy macrophage-like cells, respectively, because cells within plaques change shape and lineage markers, precluding accurate assessment of cell origin (2). All three senescent cell types were efficiently eliminated by GCV (FIG. 1A).

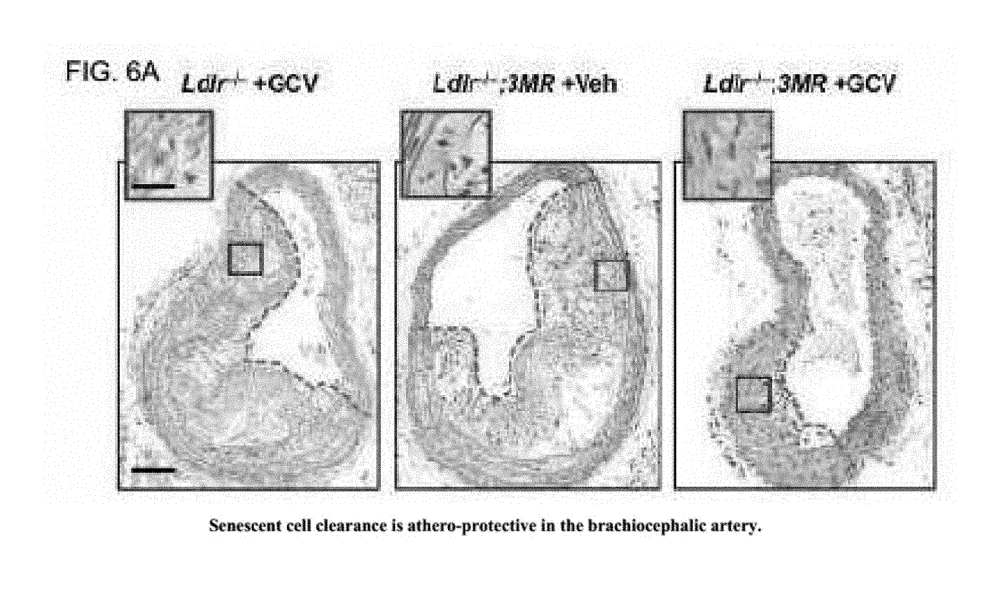

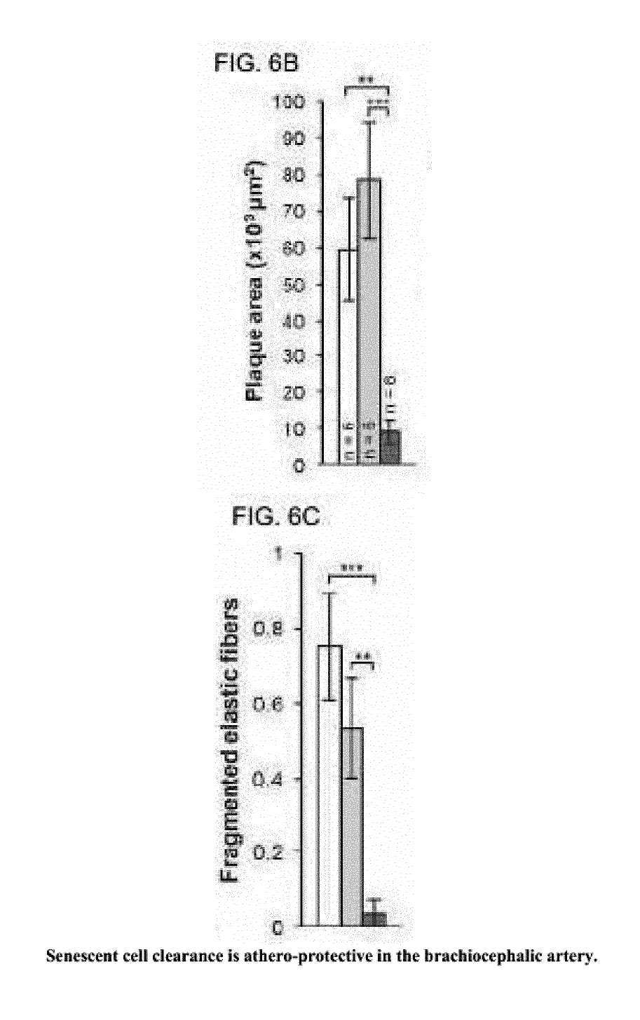

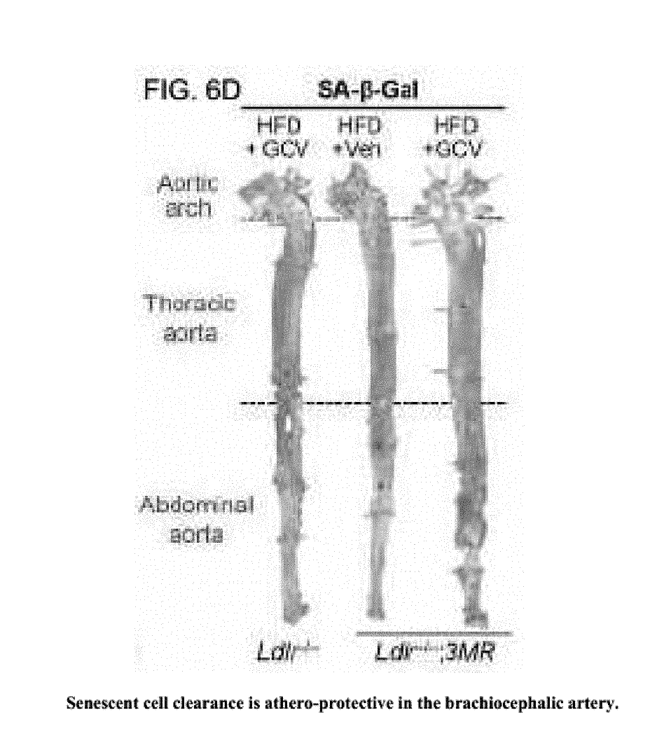

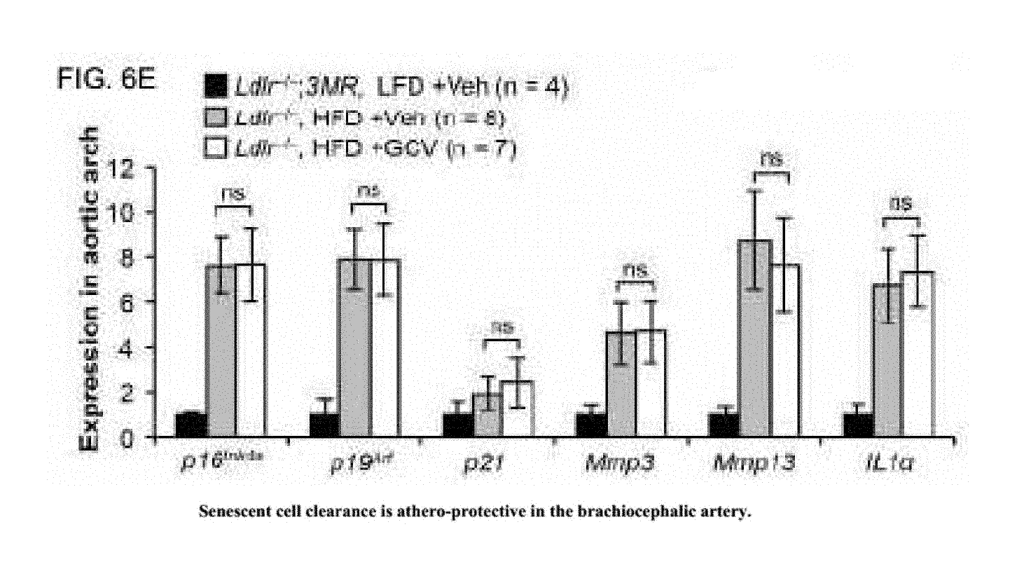

To assess the impact senescent cells have on plaque development, 10-week-old Ldlr^{-/-};3MR mice were placed on a HFD for 88 days and simultaneously treated them with GCV or vehicle during this period (FIG. 1B) to intermittently remove p16^{Ink4a}-positive cells. Ldlr^{-/-} mice on a HFD treated with GCV were included to control for potential effects of GCV independent of 3MR expression. En face staining of descending aortas with Sudan IV revealed that plaque burden was ˜60% lower in GCV-treated Ldlr^{-/-};3MR mice than in vehicle-treated Ldlr^{-/-};3MR or GCV-treated Ldlr^{-/-} mice owing to decreases in both plaque number and size (FIG. 1C). Similarly, GCV-treated Ldlr^{-/-};3MR mice showed reduced plaque burden and destruction of aortic elastic fibers beneath the neointima in the brachiocephalic artery (FIG. 6A-C), a site that rapidly develops advanced atherosclerotic plaques (17).

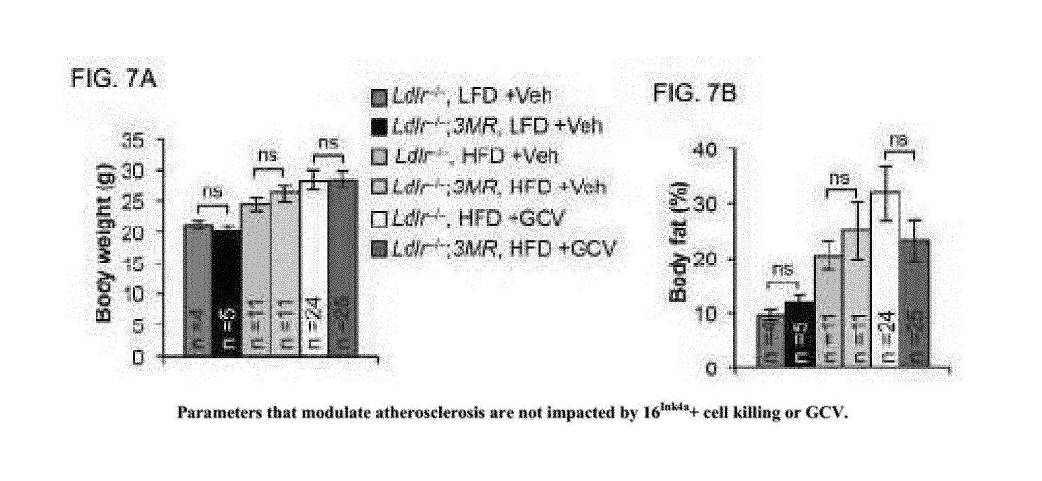

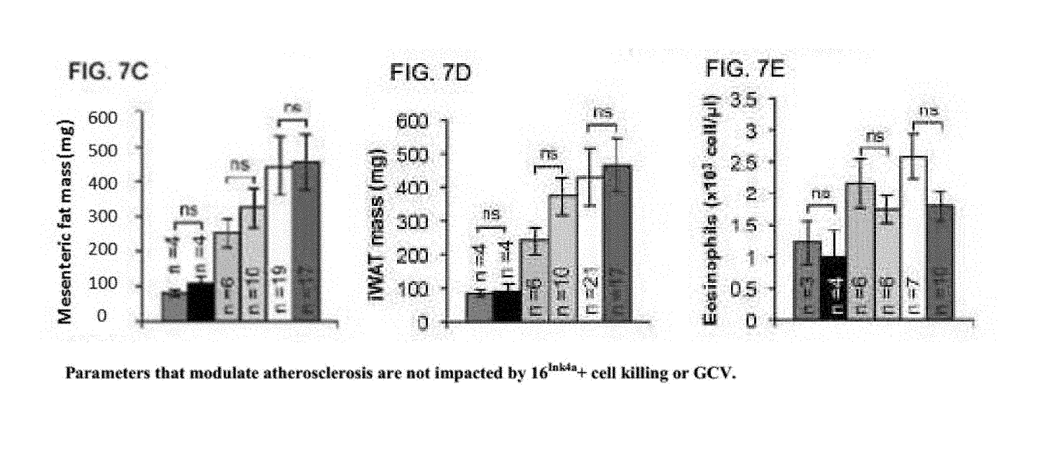

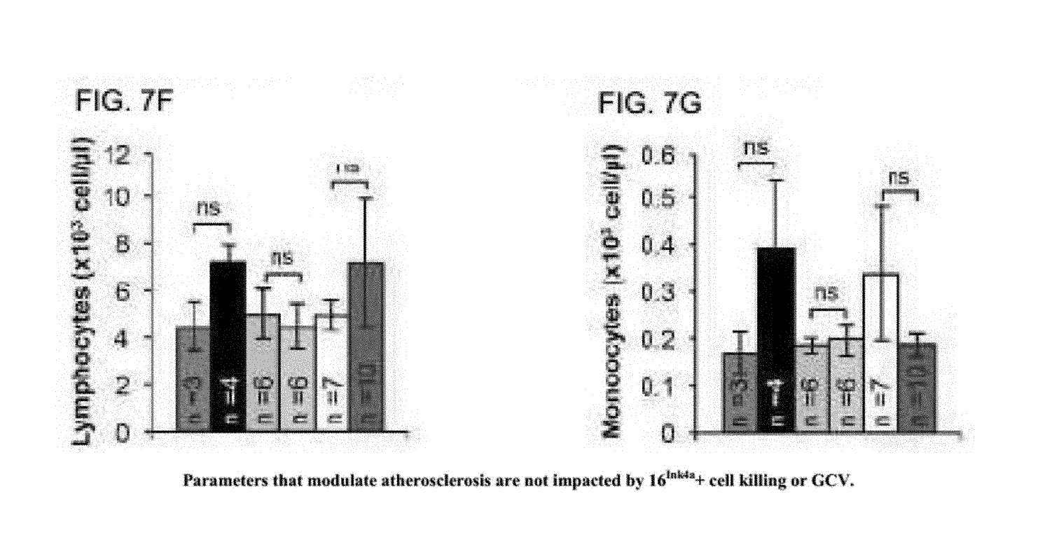

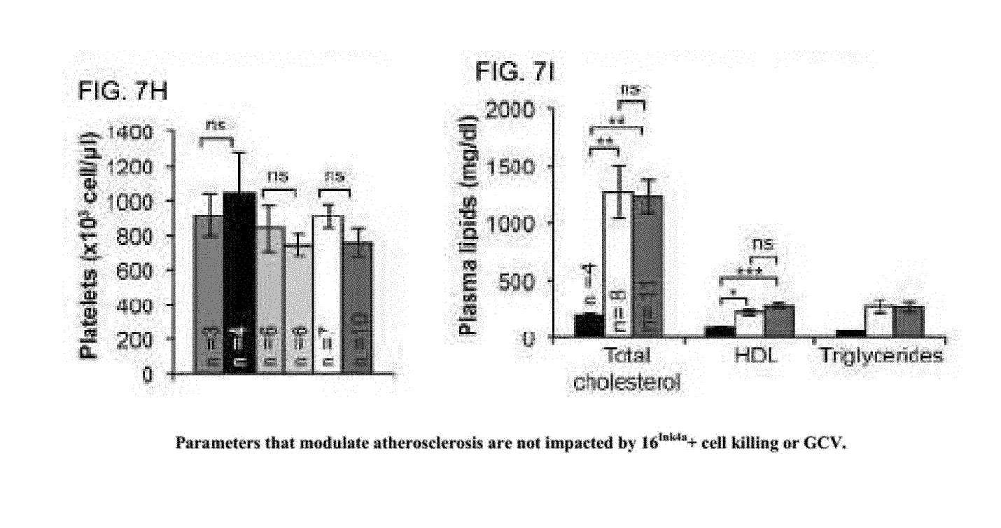

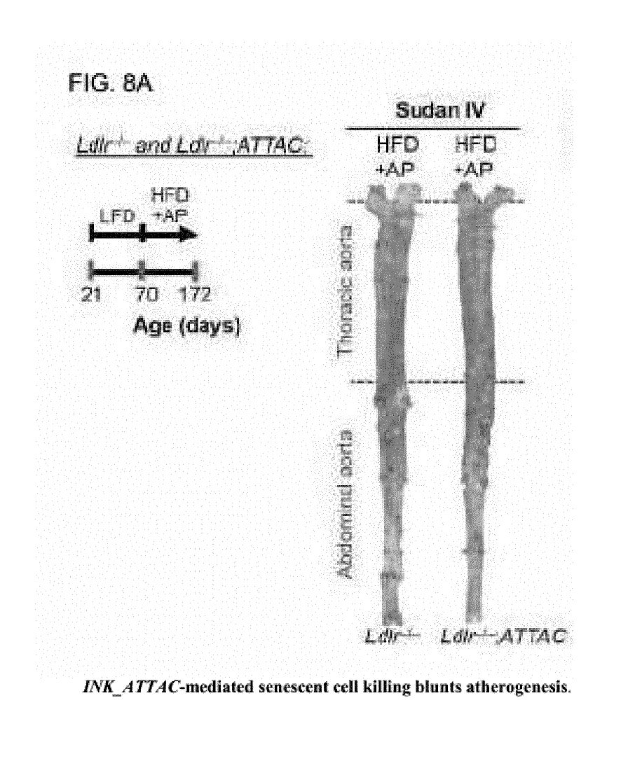

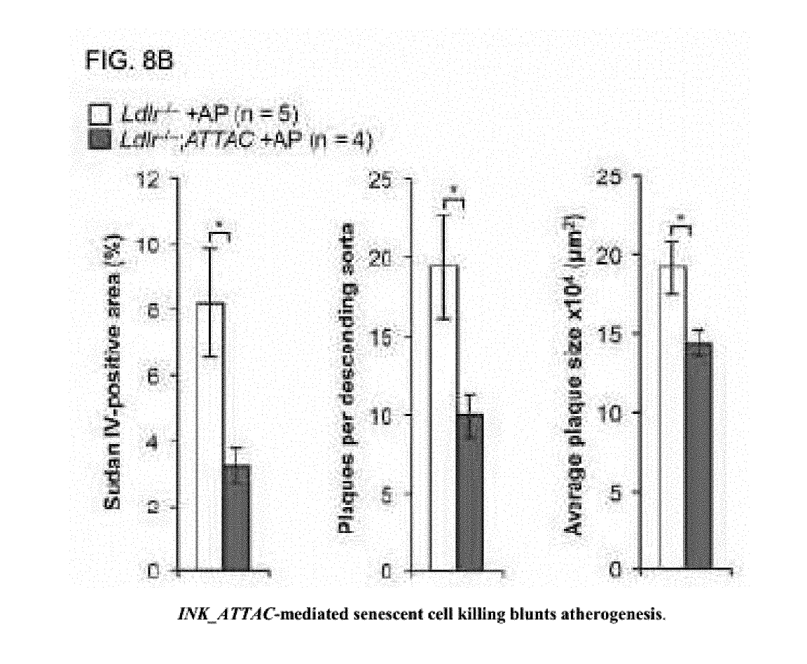

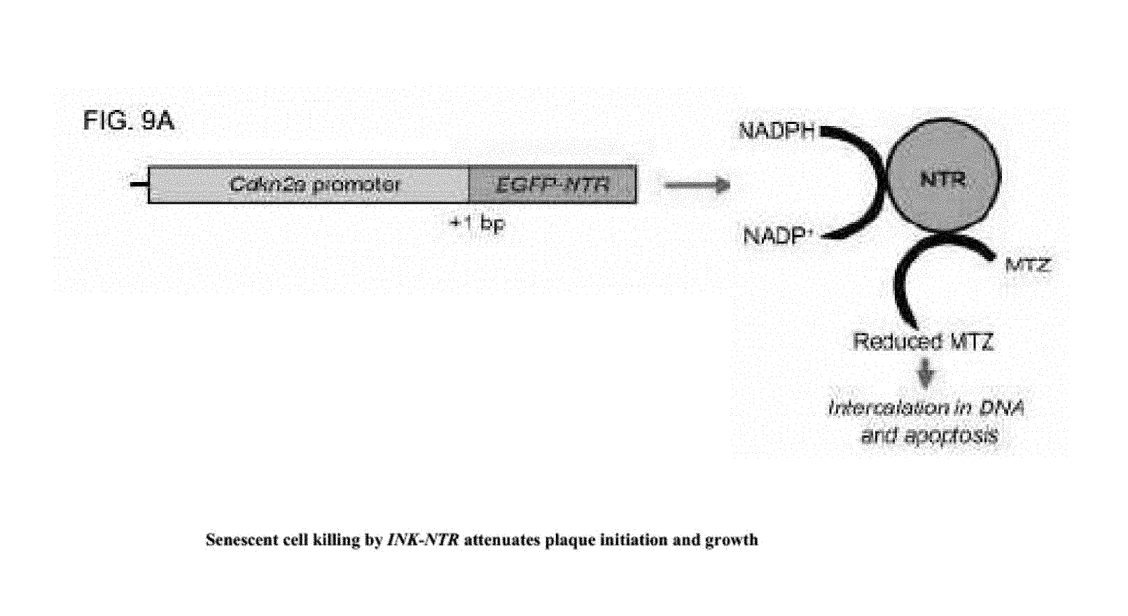

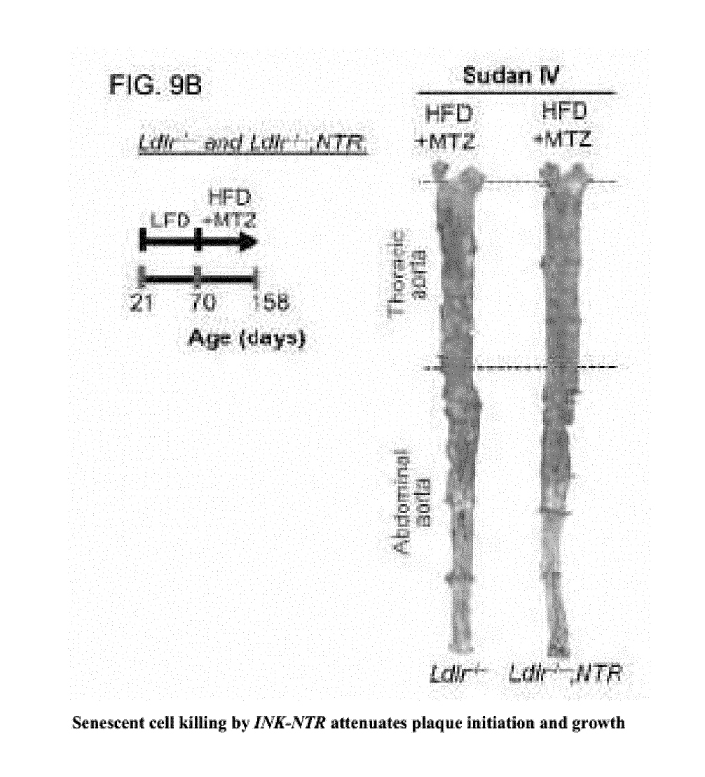

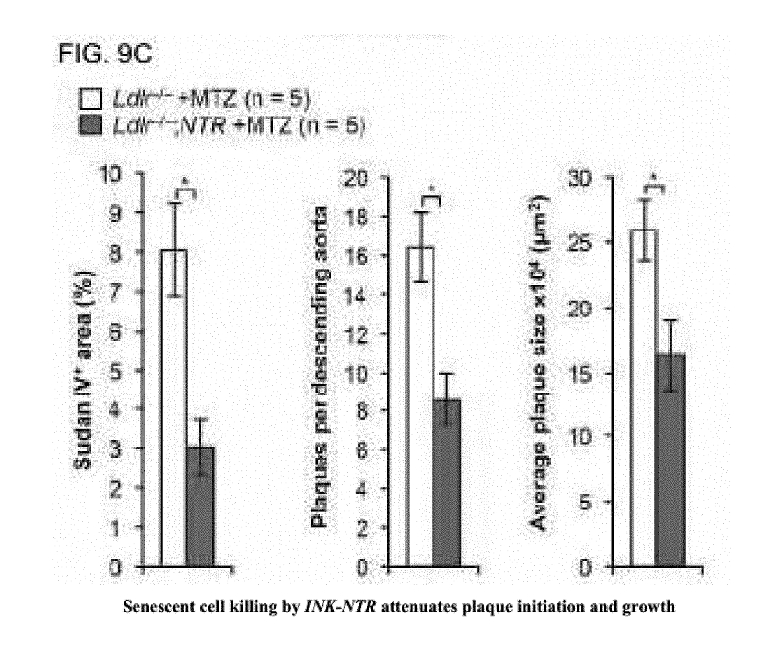

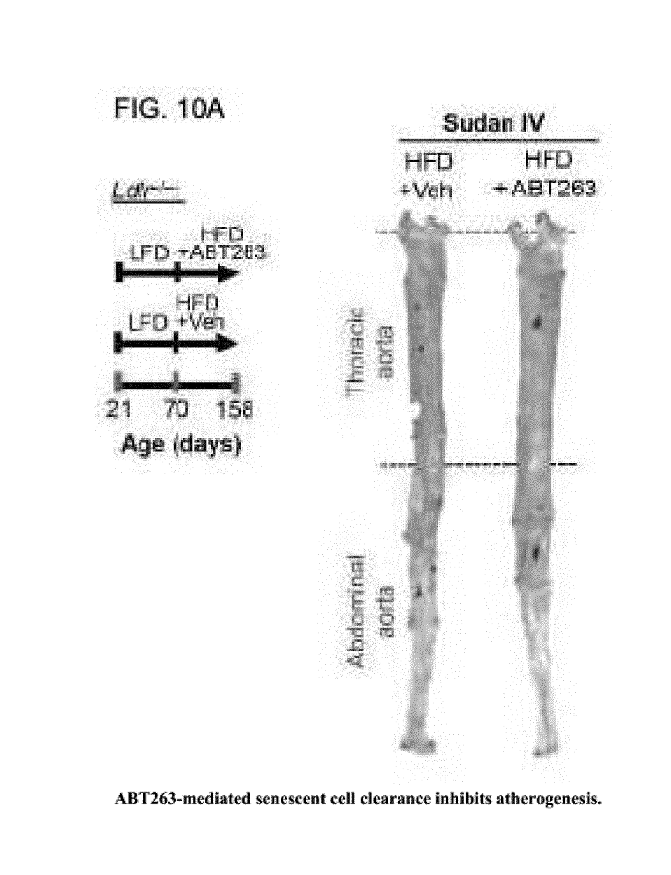

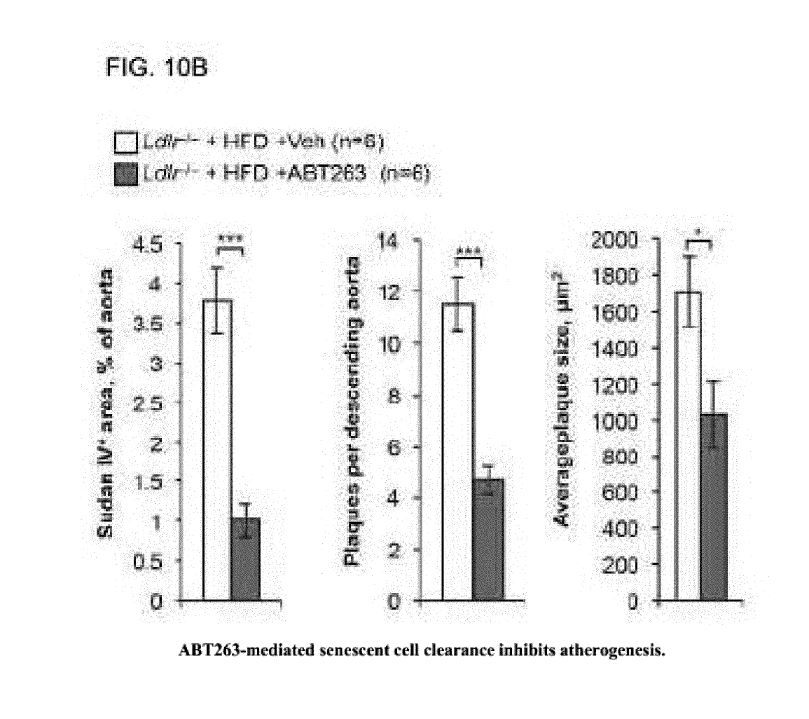

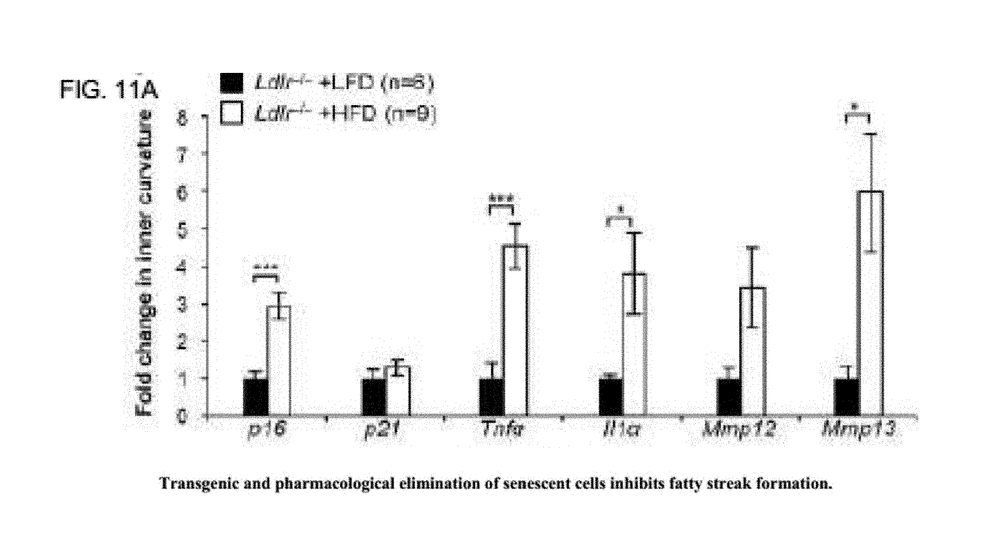

GCV-treated Ldlr^{-/-};3MR mice expressed lower amounts of p16^{Ink4a} mRNA and other senescence marker mRNAs in aortic arches than vehicle-treated Ldlr^{-/-};3MR mice, confirming that p16^{Ink4a+} senescent cells were efficiently cleared by GCV (FIG. 1D). Importantly, 3MR expression, as measured by qRT-PCR analysis of mRFP transcripts, increased in HFD fed mice, but remained at baseline levels with GCV treatment. Complementary en face SA-β-Gal staining of aortas confirmed that p16^{Ink4a}-positive senescent cells were effectively cleared (FIG. 6D). GCV-treatment of Ldlr^{-/-} mice did not alter SA-β-Gal staining or other senescence markers (FIG. 6D, E). GCV-treated Ldlr^{-/-} and Ldlr^{-/-};3MR mice did not differ in body weight, fat mass, and fat deposit weight (FIG. 7A-D). Circulating monocytes, lymphocytes, platelets, and neutrophils, all of which are involved in atherogenesis, were unaffected (FIG. 7E-H). Atherogenic lipids in the blood of GCV-treated Ldlr^{-/-} and Ldlr^{-/-};3MR mice and vehicle-treated Ldlr^{-/-};3MR mice were all highly elevated compared to LFD-fed controls, with no differences between the distinct HFD-fed cohorts (FIG. 7I). Thus, the athero-protective effect in GCV-treated Ldlr^{-/-};3MR mice is due to the killing of p16^{Ink4a+} senescent cells rather than changes in feeding habits, blood lipids or circulating immunocytes. Importantly, reductions in plaque burden, number and size observed with p16-3MR were reproducible with two independent transgenic systems designed to kill p16^{Ink4a}-positive senescent cells through distinct mechanisms, INK-ATTAC (FIG. 8) (18, 19) and INK-NTR (FIG. 9), as well as the senolytic drug ABT263 (FIG. 10) (20).

To investigate how senescent cells drive plaque initiation and growth, focus was placed on atherogenesis onset at lesion-prone sites of the vasculature (21). After just nine days on an atherogenic diet, Ldlr^{-/-} mice had overtly detectable fatty streak lesions solely in the inner curvature of the aortic arch (FIG. 2A). Surprisingly, these early lesions were highly positive for SA-β-Gal activity (FIG. 2A). By contrast, Ldlr^{-/-} mice containing 3MR and treated daily with GCV during the 9-day HFD-feeding period had low SA-β-Gal-activity and much smaller fatty streaks (FIG. 2A). Histological examination by TEM of the SA-β-Gal-stained samples revealed that fatty streaks of HFD-fed Ldlr−/− mice consisted of foci of foam cell macrophages arranged in mono- or multilayers (FIG. 2B, C). The lesions had intact elastic fibers and no fibrous cap. X-Gal crystals were detectable exclusively in foam cell macrophages, irrespective of lesion size (FIG. 2B, D). Foam cell macrophages in foci of 9-day lesions of Ldlr^{-/-};3MR mice receiving daily, high-dose GCV were rarely arranged in multilayers and had a much lower incidence of crystals (FIG. 2C. D). High SA-β-Gal activity in fatty streaks correlated with increased levels of p16^{Ink4a} and various other senescence markers, including Mmp3, Mmp13, Il1α and Tnfα (FIG. S 7A). Nine-day treatment of HFD fed Ldlr^{-/-} mice with ABT263 confirmed that senolysis reduces atherogenesis onset (FIG. 11B).

To determine how senescent foamy macrophages contribute to early atherogenesis, 9-day fatty streaks were established in Ldlr^{-/-} and Ldlr^{-/-};3MR mice and then administered high-dose GCV for 3 days while continuing HFD feeding. Short-term clearance of senescent cells markedly reduced streak size and SA-β-Gal positivity (FIG. 2E). TEM showed that cleared foci were drastically remodeled, with acellular debris retained in the subendothelium and few foamy macrophages containing X-Gal crystals (FIG. 11C, D). qRT-PCR revealed a stark reduction in p^{16Ink4a} and SASP components, including Mmp3, Mmp13, Il1α and Tnfα, as well as two key molecular drivers of monocyte recruitment, the chemokine Mcp1 and the leukocyte receptor Vcam1, whose expression is driven in part by Tnfα (FIG. 2F). These data suggests that subendothelial senescent foamy macrophages arising in early lesions enhance Tnfα-mediated Vcam1 expression as well as the Mcp1 gradient to perpetuate monocyte recruitment from the blood. Next, the role of senescent cells in the maturation of benign plaques to volatile lesions was examined. Although mouse models for atherosclerosis do not develop clinical symptoms associated with plaque ruptures, plaque maturation in these mice can be studied using surrogate markers of plaque instability, including fibrous cap thinning (22, 23), decreased collagen deposition, elastic fiber degradation and plaque calcification (24).

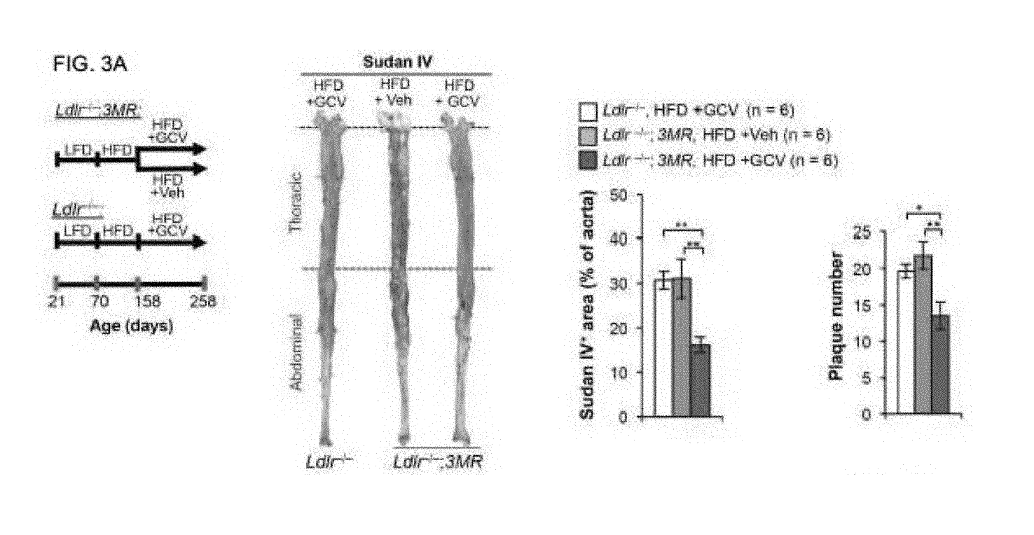

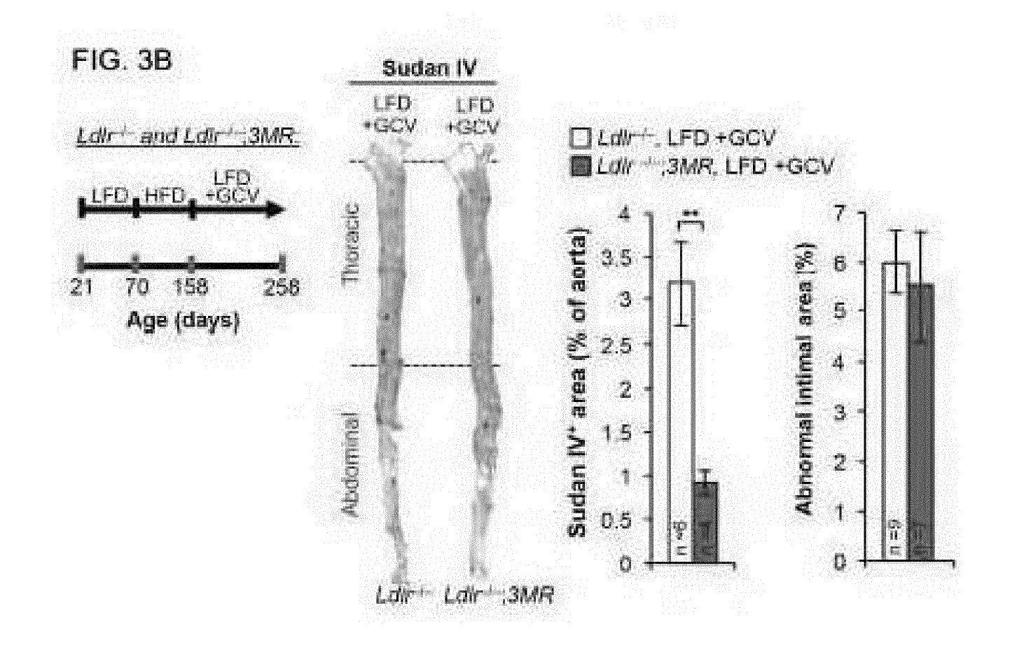

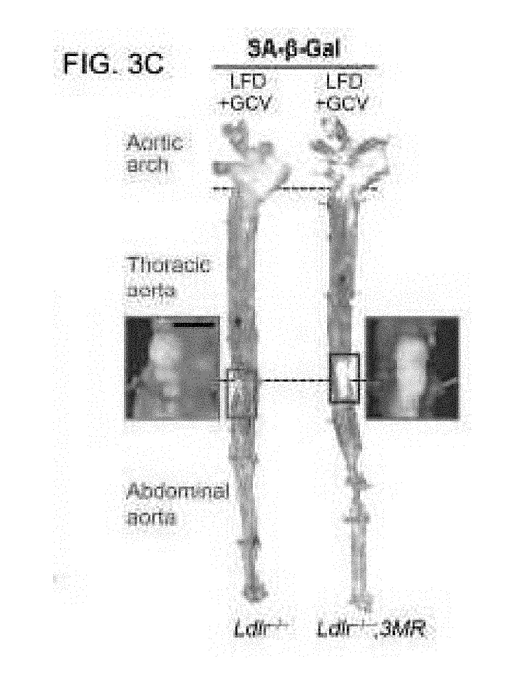

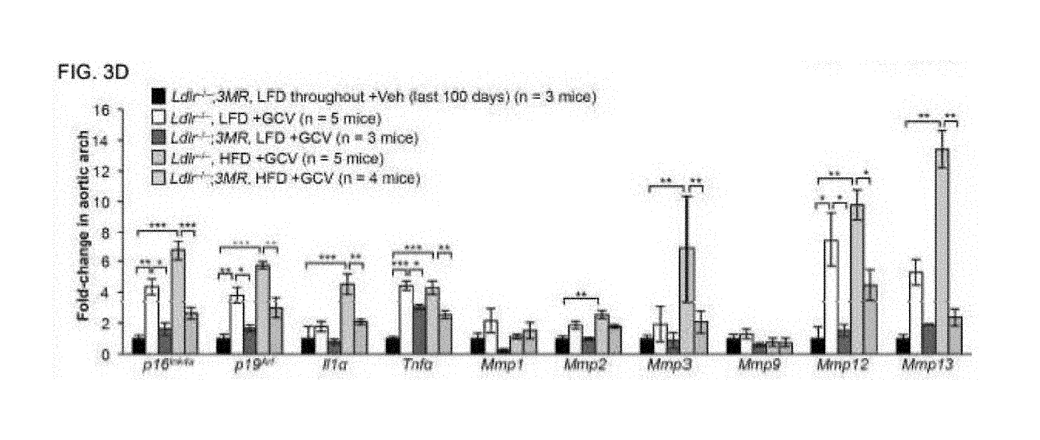

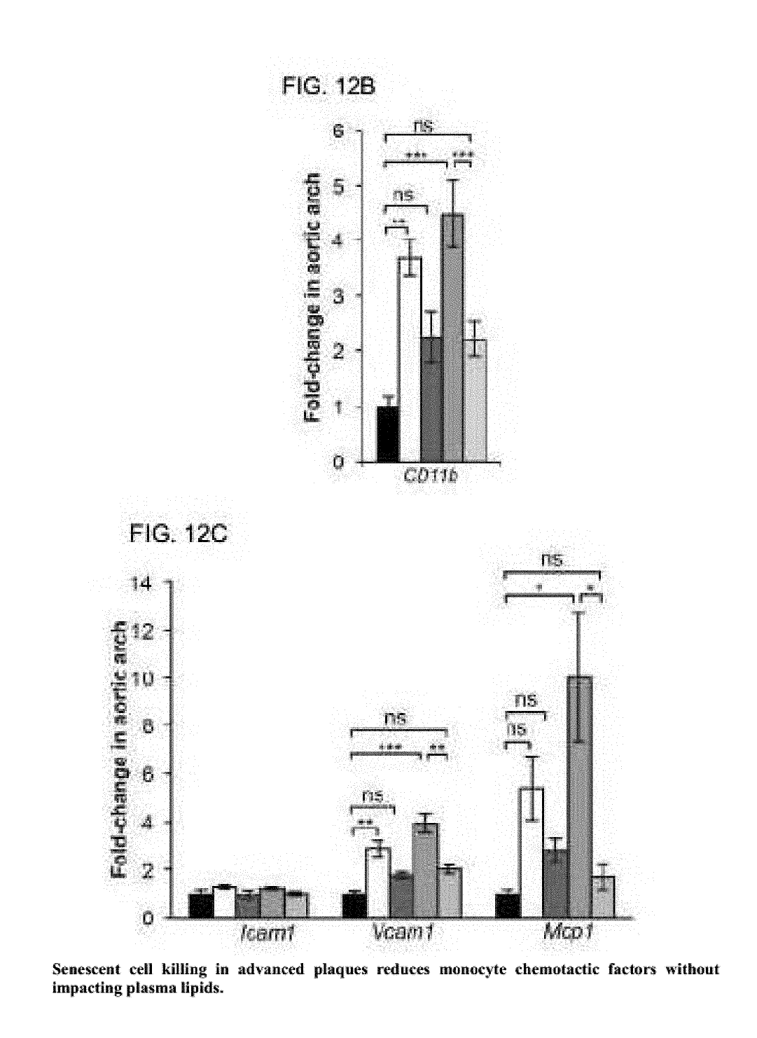

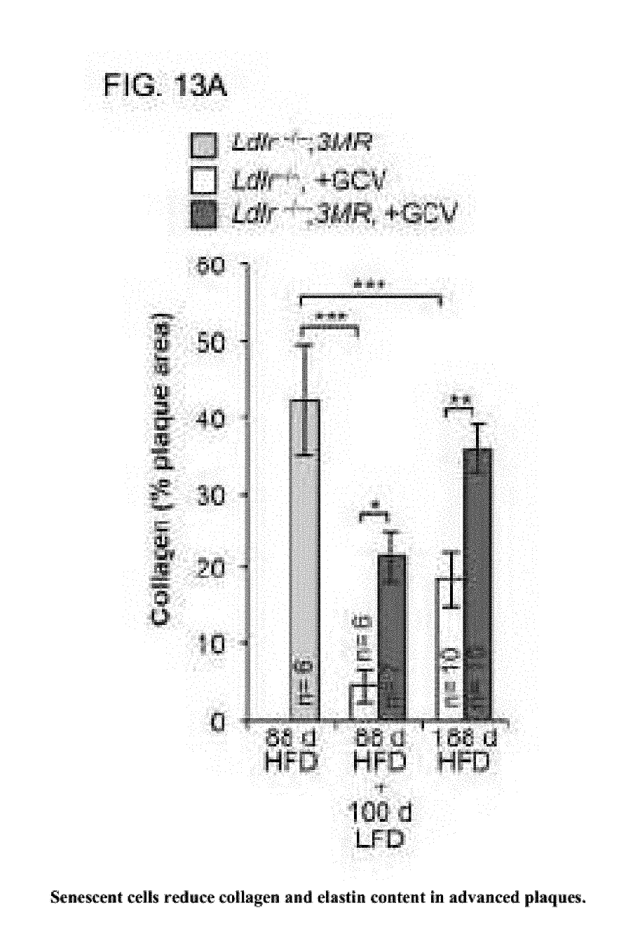

To assess the effect of senescent cell clearance on the maturation of existing plaques, late-disease senescent cell clearance protocols were employed in which Ldlr^{-/-} and Ldlr^{-/-};3MR mice were placed on HFD for 88 days to create established plaques, followed by 100 days of GCV treatment. During GCV treatment, mice were fed a HFD or LFD to promote continued plaque advancement or stasis, respectively (FIG. 3A and FIG. 12A). Ldlr^{-/-};3MR mice maintained on the HFD and receiving GCV showed attenuated disease progression, as evidenced by a lower plaque number and size compared to GCV-treated Ldlr^{-/-} or vehicle-treated Ldlr^{-/-};3MR controls (FIG. 3A). While plaques of GCV-treated Ldlr^{-/-};3MR mice on a LFD had markedly reduced Sudan IV staining compared to plaques of control mice, the lesion-covered aortic area did not change (FIG. 3B), even though 3MR-mediated senescent cell killing was confirmed by SA-β-Gal staining and qRT-PCR for senescence markers (FIG. 3C, D). Senescent cell clearance reduced expression of the inflammatory cytokines (FIG. 3D) and monocyte recruitment factors, irrespective of diet (FIG. 12B, C). Importantly, GCV treatment decreased expression of matrix metalloproteases linked to plaque destabilization, including Mmp3, Mmp12 and Mmp13 (25, 26) (FIG. 3D), suggesting that senescent cell elimination stabilizes the fibrous cap.

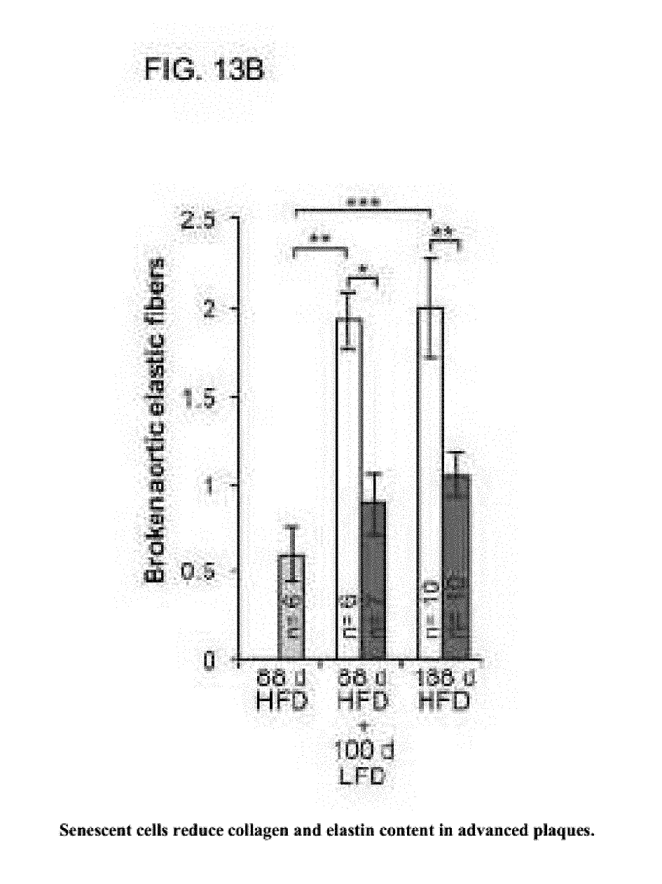

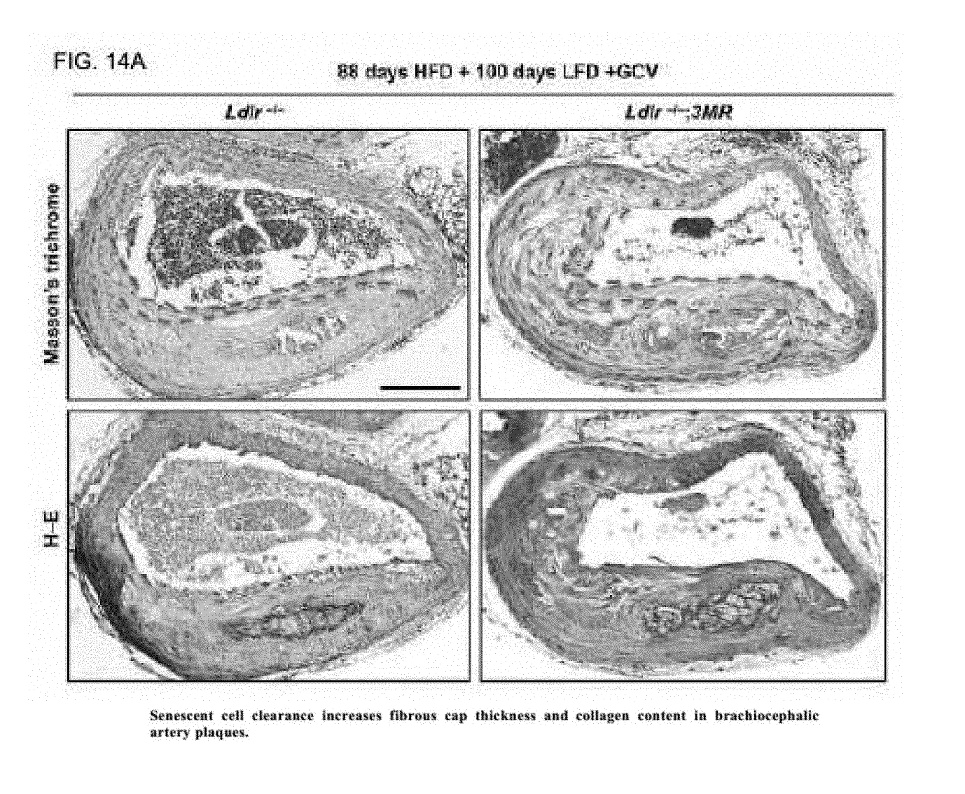

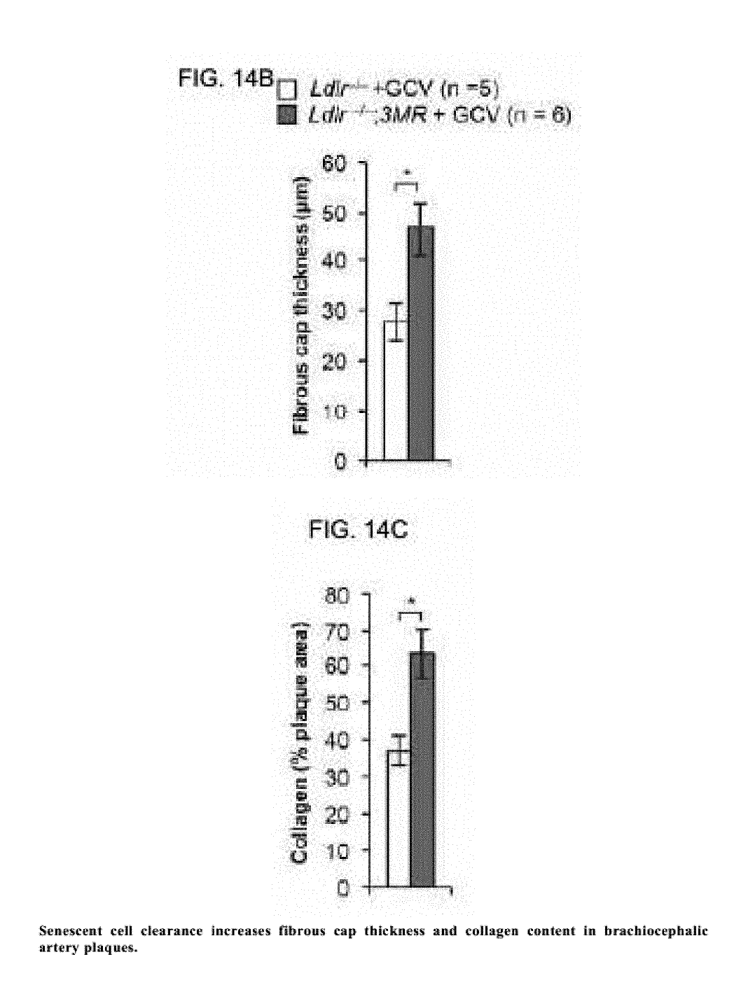

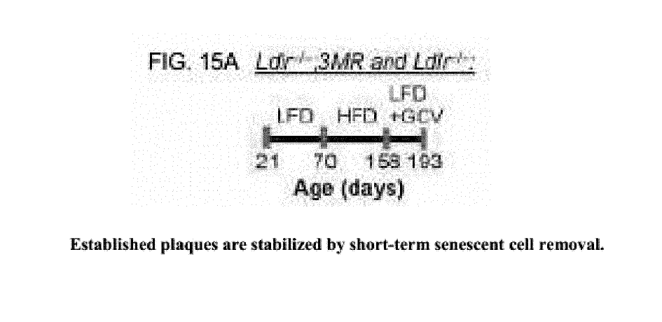

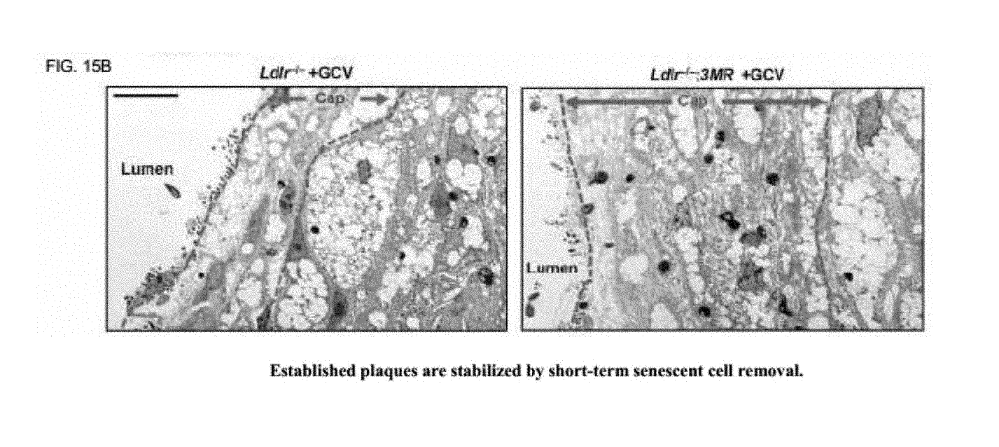

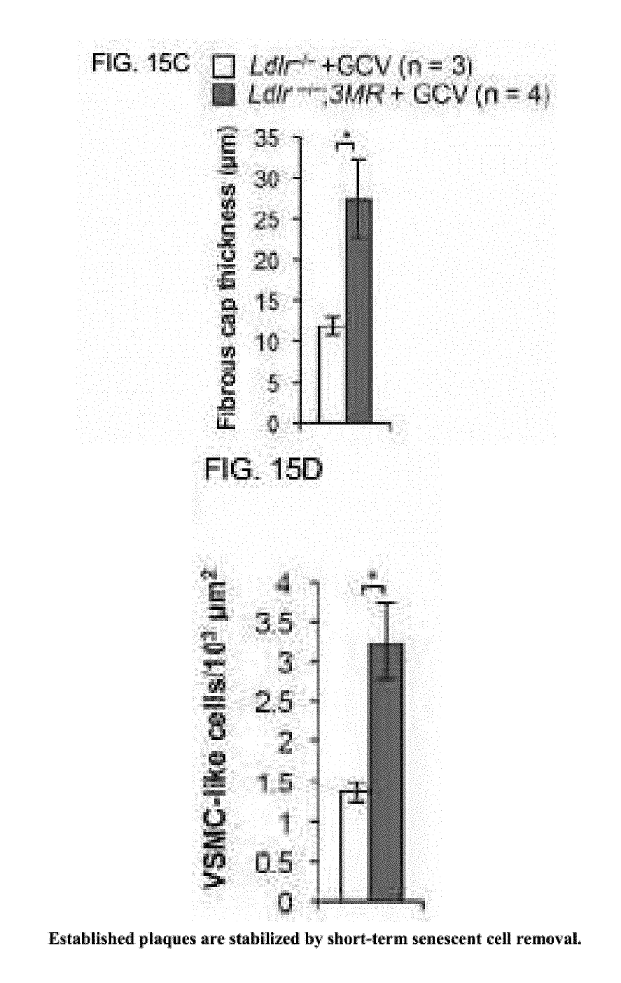

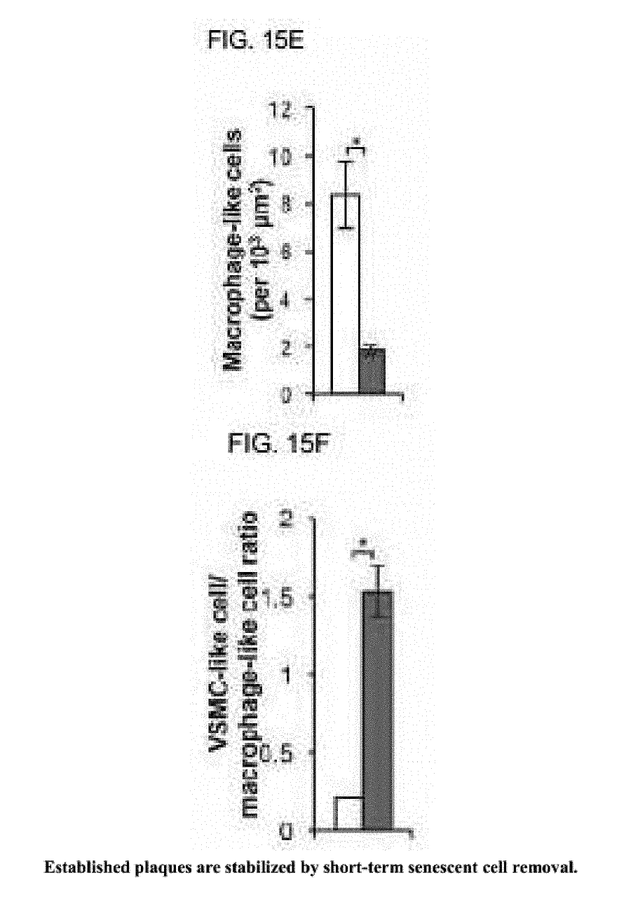

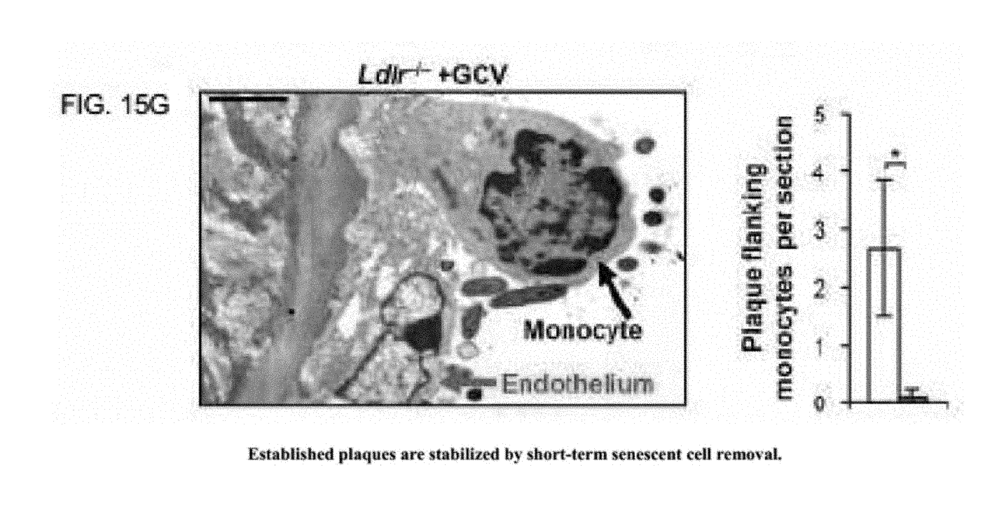

To investigate this and other features of plaque maturation, histopathology was conducted on plaques collected from the above cohorts. Descending aorta plaques of Ldlr^{-/-} mice fed a HFD for 88 days showed reduced cap thickness, diminished collagen content by Masson's trichrome staining, and more disrupted aortic elastic fibers (by Voerhoffvon Gieson-staining) when mice were left for an additional 100 days on HFD, compared to LFD (FIG. 4A, B and FIG. 13). In contrast, all these markers of plaque instability were decreased with clearance of p16^{Ink4a+} cells regardless of diet during the 100-day GVC treatment period. Similarly, clearance of p16^{Ink4a+} cells increased cap thickness and collagen content in brachiocephalic arteries from mice reverted to LFD (FIG. 14). These studies were extended by switching Ldlr^{-/-};3MR and Ldlr^{-/-} mice after 88 days of HFD feeding to a LFD with GVC injections for 35 days (FIG. 15A). In this experiment, senescent cell elimination preserved fibrous cap thickness (FIG. 15B, C). Furthermore, lesional foamy macrophage-like cell content was reduced while VSMC-like cell content increased (FIG. 15D, E), resulting in plaques with a higher VSMC-like/macrophage-like cell ratio, a marker for greater stability (FIG. 15F) (27). Numbers of circulating monocytes adhering to plaque-adjacent endothelium were substantially reduced upon clearance (FIG. 15G), supporting our conclusion from early fatty streak experiments that enhanced monocyte chemotaxis may partially explain the proatherogenic nature of senescent cells. These results strongly suggest that eliminating p16^{Ink4a+} cells promotes plaque stability.

To further investigate the mechanism by which senescent cells drive atherogenesis, the possibility that senescent cells in plaques express proatherogenic factors was tested. Lesion bearing tissue from HFD-fed Ldlr^{-/-};ATTAC mice was dissected and single cell suspensions were prepared. p16^{Ink4a}-dependent expression of GFP by ATTAC was exploited to collect GFP⁺ senescent and GFP non-senescent cell populations for analysis by qRT-PCR (FIG. 4C). Indeed, senescent cells expressed a broad spectrum of proatherogenic factors, including Il1α, Tnfα, Mmp3, Mmp12, and Mmp13, Mcp1 and Vcam1 (FIG. 4C). A subset of these factors was expressed at markedly elevated levels compared to non-senescent cells, including Il1α, Mmp12, Mmp13 and Mcp1.

Using both transgenic and pharmacological approaches to clear p16^{Ink4a}-positive cells without interfering with the senescence program, it was shown that senescent cells are uniformly deleterious throughout atherogenesis. Very early fatty streaks contain abundant senescent foam cell macrophages, which create an environment conducive to further lesion growth by upregulating inflammatory cytokines and monocyte chemotactic factors. Removing p16^{Ink4a}-positive foamy macrophages from fatty streaks led to marked lesion regression. In contrast, advanced plaques contain three morphologically distinct senescent cell types that not only drive lesion maturation through inflammation and monocyte chemotaxis, but also promote extracellular matrix degradation. While clearing senescent cells did not regress advanced lesions, it does arrest maladaptive plaque remodeling processes including fibrous cap thinning, a risk factor for plaque instability. Furthermore, senescent cells in lesions show heightened expression of key SASP factors and effectors of inflammation, monocyte chemotaxis, and proteolysis, including Il1α, Mcp1, Mmp12 and Mmp13. These data suggest that senescent cells can directly influence core proatherogenic processes through specific secreted factors.

By comparison, other factors such as Mmp3, Tnfα, and Vcam1 are reduced with senescent cell clearance but not significantly enriched in p16^{Ink4a}-positive cells, implying that senescent cells also can influence the proatherogenic milieu indirectly. Collectively, our results show that senescent cells drive atherosclerosis at all stages through paracrine activity and raise the possibility that removal of these cells could contribute to therapeutically managing atherosclerosis.

Demonstration that Treatment with a Senolytic Agent Stabilizes or Increases Fibrous Cap Thickness in Advanced Atheromas

Atherosclerosis secondary to dyslipidemia is the primary risk factor for complications of cardiovascular disease, including strokes, myocardial infarction, and other ischemic end-organ damage. Previously, senescent intimal foam cells have been shown to accumulate from the earliest stages of atherogenesis and drive disease progression, including plaque growth and destabilization. The transformation of benign fibroatheromas into clinically unstable lesions is caused by thinning of the protective fibrous cap, a vascular smooth muscle cell (VSMC) and extracellular matric (ECM)-rich layer that overlays the plaque and separates pro-coagulant plaque contents from the circulation. Enzymes, including metalloproteases, produced in the senescent cell secretome (senescence-associated secretory phenotype, or SASP) degrade collagen and elastin in the fibrous cap, resulting in fibrous cap thinning and plaque destabilization. Data provided elsewhere in this disclosure show that use of a transgenic model of senescent cell clearance (p16-3MR) in a mouse model of atherosclerosis (low-density lipoprotein receptor knockout mice; Ldlr−/− fed a high-fat diet) blocks fibrous cap thinning in descending aorta plaques. The following study was done to determine whether the use of a pharmacological senescent cell killing (senolytic) drug would recapitulate these results or add benefit to the current standard of care in treating heart disease, namely, lipid normalization through statin treatment.

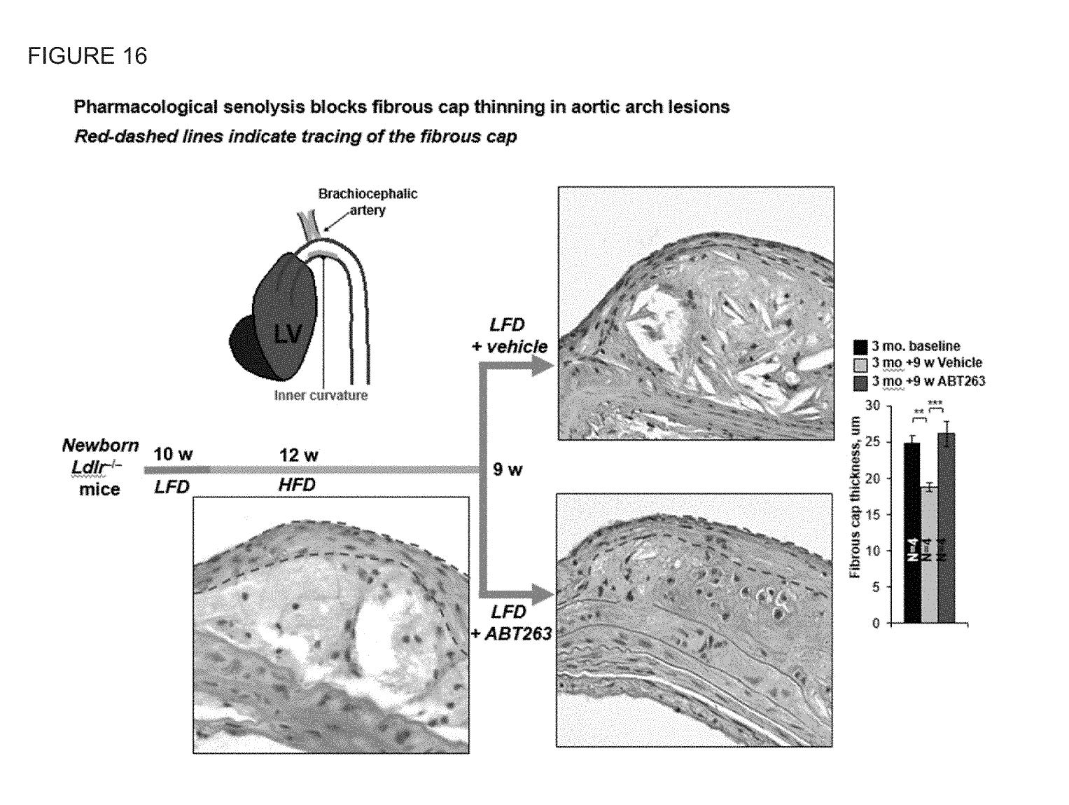

Ldlr−/− mice were placed on a high-fat diet (HFD) for 3 months to develop mature, thick-cap fibroatheromas in the aortic arch and brachiocephalic artery. Then, we concurrently switched the mice to a low-fat diet (LFD) to imitate lipid-normalizing statin treatment (standard of care in treating heart disease caused by dyslipidemia) and treated these mice with the Bcl-2/Bcl-X1 inhibitor ABT-263 (navitoclax) in order to kill senescent intimal foam cells in the atheromas or a vehicle control. Specifically, we administered ABT263 or vehicle once-per day intraperitoneally at a dose of 100 mg/kg for 9 weeks (3 cycles consisting of 7 days of treatment followed by 14 days of rest). At the end of this regimen, mice were euthanized and the vascular tree dissected for histological analysis. Baseline lesions from Ldlr−/− mice fed the HFD for 3 months were also collected. We found that the aortic arch fibrous cap thins by ˜25% during the 9-week LFD feeding interval despite plaque cross-sectional area remaining the same, indicating that even when disease is effectively stabilized due to lipid normalization, plaque destabilization still occurs (FIG. 16). In contrast, treatment with ABT263 completely blocks fibrous cap thinning. Staining with the senescence marker SA β-gal confirmed a significant removal of senescent cells versus vehicle control, as well as a significant reduction in total foam cell macrophages, one of the major intimal foam cell types that undergoes senescence during atherogenesis.

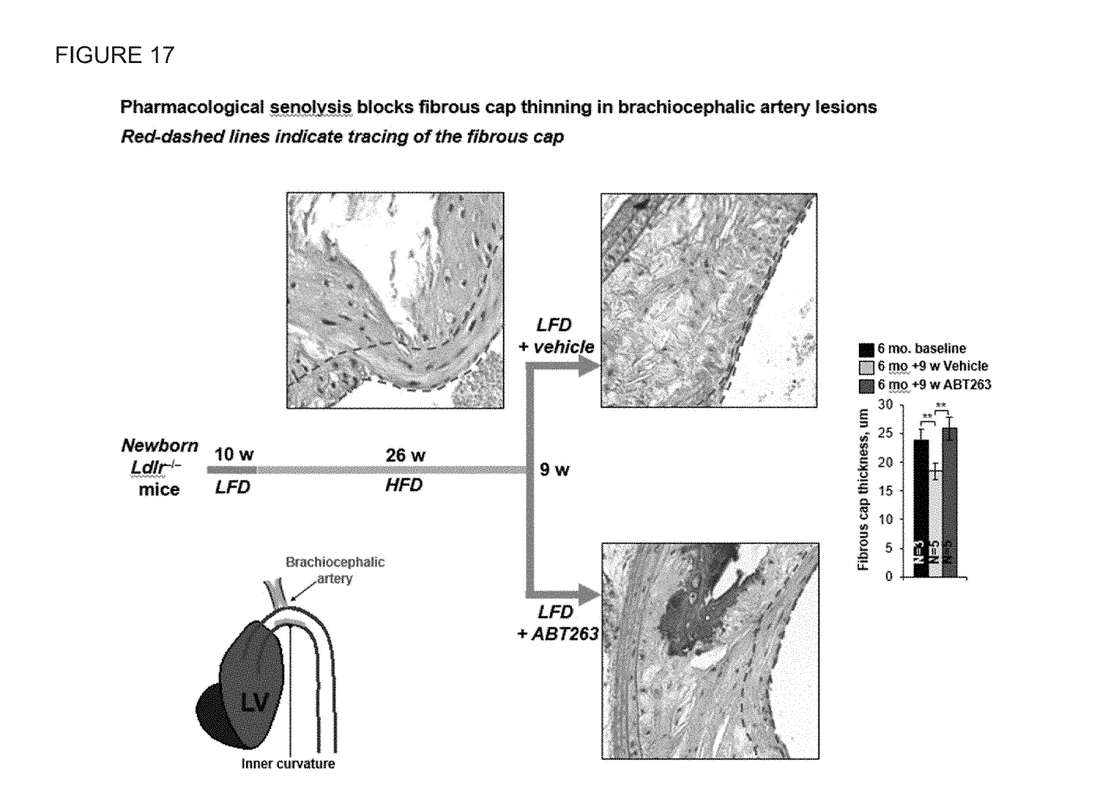

We further tested the ability of ABT263 to stabilize the fibrous cap through a similar experiment in which we produced extremely advanced, thin-cap fibroatheromas in the aortic arch of Ldlr−/− mice with 6 months of HFD feeding followed by 9 weeks of LFD to arrest lesion growth. From 3 to 6 months of HFD feeding, the aortic arch fibrous cap thins by ˜42% and does not thin further during an additional 9 weeks of LFD with vehicle administration (FIG. 17). In contrast, if ABT263 is administered during the 9 weeks of LFD feeding, the fibrous cap increases in thickness by ˜29%. This result indicates that pharmacological senolysis is not only capable of blocking fibrous cap thinning, but can, in extremely thin-capped fibroatheromas, actually thicken a thin fibrous cap.

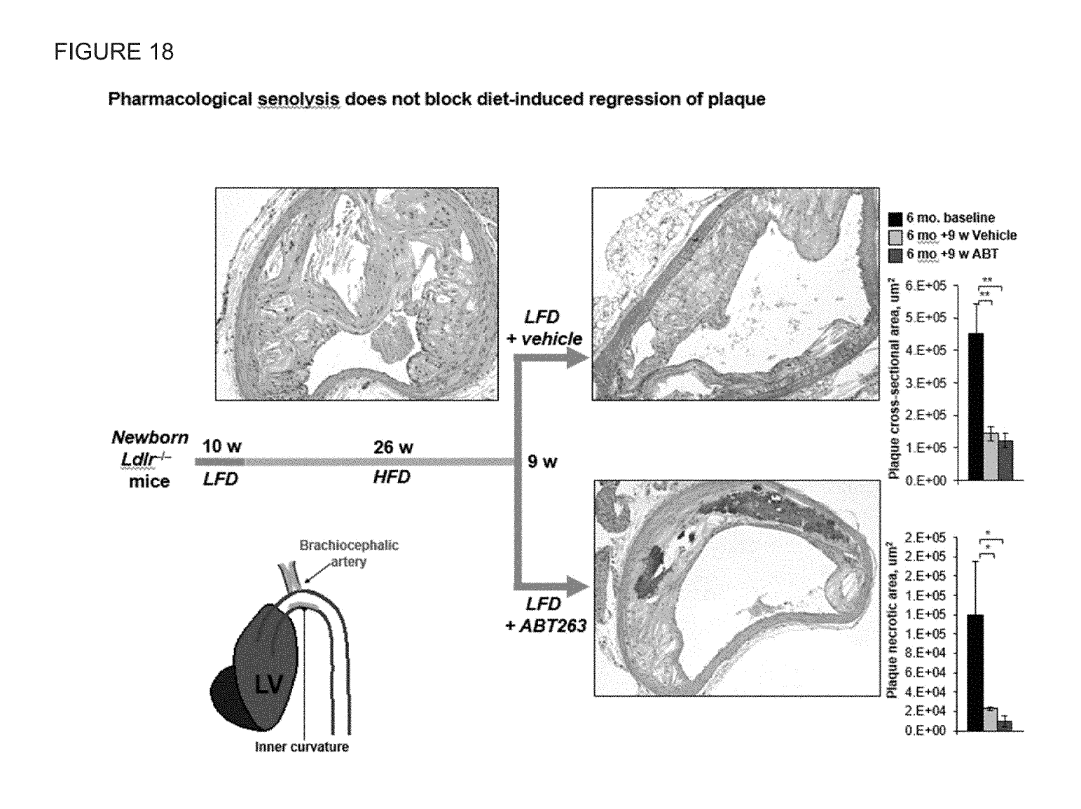

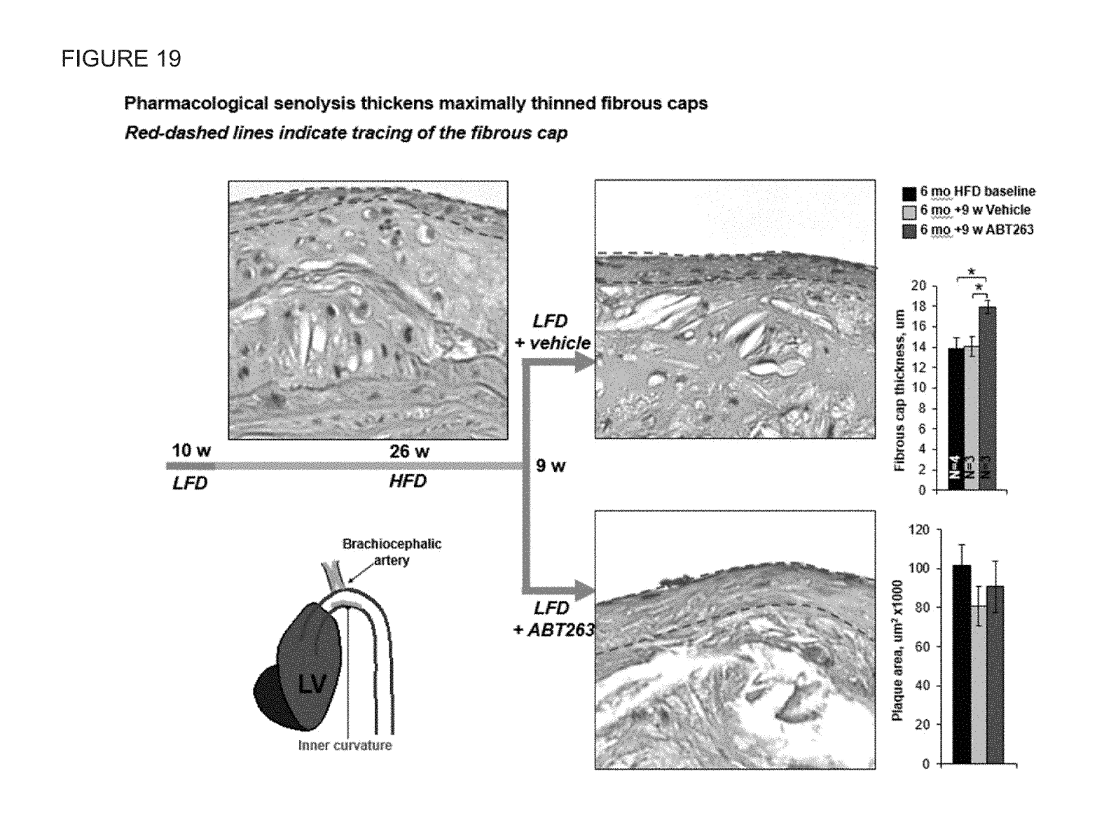

We additionally examined fibrous cap thickness at the brachiocephalic artery in these 6-month HFD mice. Here, the fibrous cap thins by ˜25% during the 9-week LFD feeding interval despite an impressive ˜66% reduction in plaque cross-sectional area (FIGS. 18 and 19). This result indicates that the fibrous cap continues to thin even in lesions that are beneficially remodeled (i.e. shrink) due to lipid normalization. In contrast, treatment with ABT263 during the 9-week feeding interval completely blocked fibrous cap thinning and, importantly, did not disrupt plaque debulking that occurred as a result of lipid normalization.

Collectively, these results reveal that senescent cell killing via the pharmacological agent ABT263 results in either blockade of fibrous cap thinning or thickening of an already thinned fibrous cap, depending on choice of anatomical site or stage of atherogenesis.

### Further Illustrations of the Action of Senolytic Agents

### Determining Senolytic Activity of Nutlin-3a

Foreskin fibroblast cell lines HCA2 and BJ, lung fibroblast cell line IMR90, and mouse embryonic fibroblasts were seeded in six-well plates and induced to senesce with 10 Gy of ionizing radiation (IR) or a 24 hr treatment with doxorubicin (Doxo). Senescent phenotype was allowed to develop for at least 7 days, at which point a cell count was made to determine the baseline number of cells. Nutlin-3a treatment was then initiated for a period of at least 9 days. Media alone or media with drug as appropriate was refreshed at least every three days. At the end of the assay time period, cells are counted. Each condition was seeded in three plate wells and counted independently. Initial cell count serves as a control to determine the induction of senescence, as compared to the last day count without nutlin treatment. Initial non-senescent cell count serves as a proxy to determine Nutlin-3a toxicity.

Foreskin fibroblast cell lines HCA2 and BJ, lung fibroblast cell line IMR90, and mouse embryonic fibroblasts were exposed to 10 Gy of ionizing radiation (IR) to induce senescence. Seven days following irradiation, the cell were treated with varying concentrations of Nutlin-3a (0, 2.5 μM, and 10 μM) for a period of 9 days, with the drug refreshed at least every 3 days. Percent survival was calculated as [cell count on day 9 of Nutlin-3a treatment/initial cell count on first day of Nutlin-3a treatment]. The results showed that Nutlin-3a reduced cell survival of senescent foreskin fibroblasts (HCA2 and BJ), lung fibroblasts (IMR90), and mouse embryonic fibroblasts (MEF), indicating Nutlin-3a is a senolytic agent.

Foreskin fibroblasts (HCA2) and aortic endothelial cells (Endo Aort) were treated with doxorubicin (250 nM) for one day (24 hours) to induce senescence. Eight days following doxorubicin treatment, Nutlin-3a treatment was initiated. HCA2 cells were exposed to Nutlin-3a for 9 days, and aortic endothelial cells were exposed to Nutlin-3a for 11 days. Media containing the compound or control media was refreshed at least every 3 days. Percent survival was calculated as [cell count on the last day of Nutlin-3a treatment/initial cell count on first day of Nutlin-3a treatment]. The results show that doxorubicin-induced senescent cells are sensitive to Nutlin-3a.

Non-senescent foreskin fibroblasts (HCA2), lung fibroblasts (IMR90), and mouse embryonic fibroblasts (MEF) were treated with varying concentrations (0, 2.5 μM, and 10 μM) of Nutlin-3a for a period of 9 days to assess Nutlin-3a toxicity. Cell counts were taken at the start (NS start) and end of Nutlin-3a treatment. The difference between counts of cells not treated with Nutlin-3a on day 9 (NS 0) and cell counts determined at day zero (NS start) reflects the cell growth over the indicated time period. The results show that Nutlin-3a treatment reduces proliferation but is non-toxic to non-senescent cells. Nutlin-3a treatment did not decrease the number of cells below the starting level, indicating an absence of toxicity. Decrease in apparent survival between NS 0 and NS 2.5 and between NS 0 and NS 10 reflects a decrease in cell growth. Without wishing to be bound by theory, Nutlin-3a may stabilize p53, leading to cell cycle growth arrest.

Non-senescent aortic endothelial (Endo Aort) cells and pre-adipocytes (Pread) were also treated with varying concentrations (0, 2.5 μM, and 10 μM) of Nutlin-3a for a period of 11 days to assess Nutlin-3a toxicity, as described above. Cell counts were taken at the start at Day 0 (NS start) and at the end of Nutlin-3a treatment (NS 0). The difference between counts of cells not treated with Nutlin-3a on day 11 (NS 0) and cell counts from NS start reflects the cell growth over the indicated time period. The results illustrate that Nutlin-3a treatment reduces proliferation but is non-toxic to non-senescent cells. As observed with fibroblasts, Nutlin-3a treatment does not decrease the number of cells below the starting level, indicating an absence of toxicity. Decrease in apparent survival between NS 0 and NS 2.5 and between NS 0 and NS 10 reflects a decrease in cell growth.

### Effect of Removal of Senescent Cells in Animal Models of Atherosclerosis

A study was done to assess the extent to which clearance of senescent cells from plaques in LDLR−/− mice with Nutlin-3A reduces plaque load. Two groups of LDLR−/− mice (10 weeks) are fed a high fat diet (HFD) (Harlan Teklad TD.88137) having 42%/o calories from fat, beginning at Week 0 and throughout the study. Two groups of LDLR−/− mice (10 weeks) are fed normal chow (−HFD). From weeks 0-2, one group of HFD mice and −HFD mice are treated with Nutlin-3A (25 mg/kg, intraperitoneally). One treatment cycle is 14 days treatment, 14 days off. Vehicle is administered to one group of HFD mice and one group of −HFD mice. At week 4 (timepoint 1), one group of mice are sacrificed and to assess presence of senescent cells in the plaques. For the some of the remaining mice, Nutlin-3A and vehicle administration is repeated from weeks 4-6. At week 8 (timepoint 2), the mice are sacrificed and to assess presence of senescent cells in the plaques. The remaining mice are treated with Nutlin-3A or vehicle from weeks 8-10. At week 12 (timepoint 3), the mice are sacrificed and to assess the level of plaque and the number of senescent cells in the plaques.

Plasma lipid levels were measured in LDLR−/− mice fed a HFD and treated with Nutlin-3A or vehicle at timepoint 1 as compared with mice fed a −HFD (n=3 per group). Plasma was collected mid-afternoon and analyzed for circulating lipids and lipoproteins.

At the end of timepoint 1, LDLR−/− mice fed a HFD and treated with Nutlin-3A or vehicle were sacrificed (n=3, all groups), and the aortic arches were dissected for RT-PCR analysis of SASP factors and senescent cell markers. Values were normalized to GAPDH and expressed as fold-change versus age-matched, vehicle-treated LDLR−/− mice on a normal diet. The data show that clearance of senescent cells with Nutlin-3A in LDLR−/− mice fed a HFD reduced expression of several SASP factors and senescent cell markers, MMP3, MMP13, PAI1, p21, IGFBP2, IL-1A, and IL-1B after 1 treatment cycle.

At the end of timepoint 2, LDLR−/− mice fed a HFD and treated with Nutlin-3A or vehicle (n=3 for all groups) were sacrificed, and aortic arches were dissected for RT-PCR analysis of SASP factors and senescent cell markers. Values were normalized to GAPDH and expressed as fold-change versus age-matched, vehicle-treated LDLR−/− mice on a normal diet. The data show expression of some SASP factors and senescent cell markers in the aortic arch within HFD mice). Clearance of senescent cells with multiple treatment cycles of Nutlin-3A in LDLR−/− mice fed a HFD reduced expression of most markers (FIGS. 24A-B).

At the end of timepoint 3, LDLR−/− mice fed a HFD and treated with Nutlin-3A or vehicle (n=3 for all groups) were sacrificed, and aortas were dissected and stained with Sudan IV to detect the presence of lipid. Body composition of the mice was analyzed by MRI, and circulating blood cells were counted by Hemavet. The data show that treatment with Nutlin-3A reduces plaques in the descending aorta by ˜45% (FIGS. 25A-C). The platelet and lymphocyte counts were equivalent between the Nutlin-3A and vehicle treated mice. Treatment with Nutlin-3A also decreased mass and body fat composition in mice fed a HFD.

The study assessed the extent to which acyclovir based clearance of senescent cells from LDLR−/−/3MR double transgenic mice improves pre-existing atherogenic disease. LDLR−/−/3MR double transgenic mice (10 weeks) and LDLR−/− single transgenic mice (10 weeks) are fed a high fat diet beginning at Week 0 until Week 12. Gancyclovir is administered to both groups of mice (25 mg/kg intraperitoneally) from weeks 12-13 and weeks 14-15. At week 16, the level of plaque and the number of senescent cells in the plaques are determined. Clearance of senescent cells with GCV in LDLR−/−/3MR double transgenic mice fed a HFD (n=10) reduces the % of the aorta covered with plaque as compared to LDLR−/− mice/HFD controls (n=−9). Clearance of senescent cells with GCV also reduced the plaque cross-sectional area in in LDLR−/−/3MR double transgenic mice fed a HFD (n=3) as compared to LDLR^{-/-} mice/HFD controls (n=5).

### Clearance of Senescent Cells Ameliorates Atherosclerosis in LDLR−/−/3MR Mice

The impact of clearance of senescent cells on the stability and size of mature atherosclerotic plaques was studied in LDLR−/−/3MR double transgenic mice. From 10 weeks of age, LDLR−/−/3MR double transgenic mice (10 weeks) and LDLR−/− single transgenic mice (control) were fed a high fat diet (Harlan Teklad TD.88137) having 42% calories from fat beginning at Week 0 until Week 12.5, when the mice were switched to normal chow diet. Both groups of mice were treated with ganciclovir from week 12.5 over the next 100 days, with each treatment cycle comprising 5 days of ganciclovir (25 mg/kg intraperitoneally daily) and 14 days off. At the end of the 100 day treatment period, the mice were sacrificed, plasma and tissues were collected, and atherosclerosis was quantitated.

Descending aortas were dissected and stained with Sudan IV to visualize the plaque lipids. Ganciclovir-treated LDLR−/−/3MR double transgenic mice had fewer atherosclerotic plaques with less intense staining than the LDLR−/− control mice fed a HFD. The % of the aorta covered in plaques as measured by area of Sudan IV staining was also significantly lower in the ganciclovir-treated LDLR−/−/3MR mice as compared to the LDLR−/− control mice.

Plaques from ganciclovir-treated LDLR−/− control and LDLR−/−/3MR mice were harvested and cut into cross-sections and stained with to characterize the general architecture of the atherosclerotic plaques. “#” indicates fat located on the outside of the aorta.

Clearance of senescent cells in ganciclovir-treated LDLR−/−/3MR mice has an effect on plaque morphology as compared to LDLR−/− control mice. The plaque from the control mice has identifiable “lipid pockets” accumulating within. The plaque from the ganciclovir treated LDLR−/−/3MR mice shows the presence of a thick fibrin cap and the absence of lipid pockets. Disruption or tear in the cap of a lipid-rich plaque is a trigger for coronary events through exposure of plaque thrombogenic components to platelets and clotting components of the blood. Plaques that grow more rapidly as a result of rapid lipid deposition and have thin fibrin caps are prone to rupture. Slowly growing plaques with mature fibrin caps tend to stabilize and are not prone to rupture. Taken together, these experiments indicate that removal of senescent cells may affect atherosclerotic plaque architecture and have a stabilizing effect.

Tissue sections of atherosclerotic aortas were prepared and stained to detect SA-β-GAL. X-GAL crystals were located in the lysosomes of lipid-bearing macrophage foam cells and smooth muscle foam cells.

### Source Materials and Techniques

C57BL/6 Ldlr−/− mice were purchased from the Jackson Laboratory (stock number 002207), crossed with previously described C57BL/6 3MR mice (15) to generate Ldlr+/−;3MR mice, which were then bred to C57BL/6 Ldlr−/− mice to produce Ldlr−/−;3MR males. Female mice used in experiments were generated by breeding Ldlr−/−;3MR males to C57BL/6 Ldlr−/− females. Experimental mice contained a single copy of the 3MR transgene. INK-ATTAC transgenic mice on a C57BL/6 background were established as described (27). These mice contain ˜13 tandem copies of the INK-ATTAC transgene integrated into a single genomic locus (27). Breeding the INK-ATTAC transgene onto the Ldlr−/− background and experimental cohort production was performed as described for Ldlr−/−;3MR.

Experimental mice were hemizygous for INK-ATTAC. INK-NTR mice were generated by replacing the FKBP-Casp8-IRES-EGFP segment of the INK-ATTAC transgene cassette with an EGFP-NTR fusion gene (NTR was amplified from E. coli BL21) (28). The transgene was injected into FVB fertilized eggs yielding 14 transgenic founders of which eight were bred onto a BubR1 progeroid background (29). BubR1H/H;INK-NTR and BubR1H/H littermates for each founder line were given ad libitum access to drinking water containing 4.5 g/l MTZ (Sigma-Aldrich) and 90 g/l sugar beginning at weaning age and were subsequently monitored for the time to onset of cataracts, kyphosis and lipodystrophy as described (18). Two transgenic lines markedly attenuate these features and one was selected for breeding to Ldlr^{-/-} mice (line 18; these mice were of a FVB×129Sv/E×C57BL/6 mixed genetic background). Experimental mice were generated by breeding Ldlr−/−;INK-NTR males to C57BL/6 Ldlr−/− females. Ldlr−/−;INK-NTR females used in experiments were hemizygous for INK-NTR and had been backcrossed to C57BL/6 for at least 3 generations.

### Atherosclerosis Induction and Senescent Cell Clearance

To induce atherosclerosis, female mice were fed an atherogenic diet consisting of 42% calories from fat (Harlan-Teklad, TD.88137) starting from 10 weeks of age. Progression studies in FIG. 1 and FIG. 5-10 used 88 days (12.5 weeks) of HFD feeding prior to sacrifice, with the exception of FIG. 9, which used 102 days of HFD. For studies using the 3MR system, 5 mg/kg ganciclovir (GCV) in PBS was delivered intraperitoneally (IP) to Ldlr−/− controls and Ldlr−/−;3MR experimental mice once daily for 5 days, followed by 14 days off on a repeating cycle for the duration of the study. PBS-injected Ldlr−/−;3MR were also put on HFD as controls for transgene-insertion effects. For studies using metronidazole, Ldlr−/− controls and Ldlr−/−;NTR experimental mice were given ad libitum drinking water containing 4.5 g/l metronidazole (Sigma-Aldrich) and 90 g/l sugar. For studies using AP20187, Ldlr−/− controls and Ldlr−/−;ATTAC experimental mice were given 2 mg/kg AP20187 via IP injection twice-weekly as described (27) for the duration of the study.

To induce fatty streaks in FIG. 2 and FIG. 11, Ldlr−/− and Ldlr−/−;3MR mice were fed HFD for 9 days with once-daily injections of 25 mg/kg GCV in PBS (referred to as high-dose GCV in the main text) or 100 mg/kg ABT263 in vehicle (PBS with 15% DMSO/7% Tween-20). To regress fatty streaks, mice were pre-fed HFD for 9 days, followed by a further 3 days of HFD with 25 mg/kg GCV in PBS delivered 3× daily.

Late-stage progression in FIG. 3-4 and FIG. 12-10 was studied by feeding HFD to Ldlr−/− and Ldlr−/−;3MR mice for 188 days, where treating (PBS vehicle or 5 mg/kg GCV) during the last 100 days on a 5 days on, 14 days off cycle. Late-stage regression in FIG. 3-4 and FIG. 12-10 was studied used 88 days of HFD followed by a switch back to non-atherogenic standard irradiated pelleted chow diet (LabDiet #5053, 13.205% calories from fat) with vehicle or GCV treatment as above. Non-atherosclerotic controls were fed this same non-atherogenic diet lifelong and treated with PBS vehicle for the last 100 days of the study.

### Blood Profiling

Prior to sacrifice, blood was collected by retro-orbital puncture using heparinized capillary tubes. Gross hematology for circulating cells was assessed by analyzing EDTA-treated whole blood using a Hemavet 950 (Drew Scientific Inc., Miami Lakes, Fla., USA). Plasma was prepared by EDTA treating whole blood followed by centrifugation at 4° C. for 15 min at 3500 g. Lipid analysis was performed by the Mayo Clinic Immunochemical Core Laboratory (ICL) using high-performance liquid chromatography (HPLC).

### SA-β-Gal Staining and Gal-EM

SA-β-Gal staining on mouse aortas was performed using a kit according to the manufacturers instructions (Cell Signaling). Whole mouse aortas were excised and stored in PBS on ice until fixation. Aortas were fixed for 15 min at RT, washed twice in PBS, and developed in staining solution for 12 h at 37° C. Electron microscopy on SA-β-Gal-stained plaques (Gal-EM) was performed as described (27). Briefly, following SA-β-Gal staining, plaques were post-fixed in Trump's fixative for 4 h at RT, followed by standard EM processing (dehydration through xylene-alcohol series, followed by osmium tetroxide staining and embedding in Epon resin). For quantification of SA-β-Gal-positive cells in FIG. 1, two non-adjacent thin sections were analyzed per descending aorta plaque, with cells bearing one or more cuboidal or needle-shaped X-Gal crystals considered to be X-Gal+. Foamy macrophage-like cells, VSMC-like cells, endothelial-like cells, and adherent monocytes were identified morphologically. Briefly, cells with roughly circular shape and the entire cytoplasm occupied with vacuoles are considered macrophage-like.

Spindle-shaped or highly ramified cells with electron-dense, largely unvacuolated cytoplasm rich in Golgi/endoplasmic reticulum are considered vascular smooth muscle-like cells. Cells localized to the plaque surface with elongated nuclei and long, thin cytoplasm are considered endothelium. These morphological assessments fully disregard cellular origin given cell-type interconversion prevalent in lesions, and thus describe cells as ‘-like’ in order to capture broad phenotypic categories. For quantification of plaque histological parameters in FIG. 14, one or more thin sections were analyzed per descending aorta plaque. Cap thickness was measured at 15 equally dispersed sites per section on 600× magnification TEM images using ImageJ. VSMC-like and macrophage-like cell content were analyzed on these same sections by manually counting all such cells at 1500× magnification and normalizing to plaque cross-sectional area. Adherent monocytes, which were only found on endothelium adjacent to plaques, were counted in two non-adjacent sections for each plaque and are presented without normalization to endothelial cell numbers because quantity of adjacent vessel wall was approximately equal between groups.

### En Face Staining for Plaques

Whole aortas were dissected clean of adventitial fat, opened, and pinned flat in 4% paraformaldehyde (PFA) for 12 h at RT as previously described (30). Staining was conducted by washing pinned aortas for 5 min with 70% ethanol, incubating in Sudan IV working solution (0.5% Sudan IV in 1:1 acetone:ethanol) for 5 min, followed by differentiating three times for 30 sec with 80% ethanol. For all experiments, control and experimental aortas were stained simultaneously. Quantification of total Sudan IV+ area was done using ImageJ and plaques were counted at 40× magnification.

### Flow Cytometry

Lesion bearing aortic arches and abdominal aortas from Ldlr−/−;ATTAC and Ldlr−/− fed a HFD for 6 months were isolated into ice-cold PBS and washed three times, before being finely minced in Hank's balanced salt solution (HBSS) with 1 mg/ml Liberase™ (Roche Life Science). Samples were incubated at 37° C. for 1 hr with inversion every ten min, and 10× trituration through a fire-polished glass pipette at 30 min and the end of digestion to disrupt the tissue. Samples were passed over a 70 μm nylon cell strainer and the filter was rinsed with 2 ml HBSS with 5% normal goat serum (NGS) to collect cells, which were pelleted at 300 g for 4 min at 4° C., and resuspended in 0.75 ml HBSS with 5% NGS. Samples were stored on ice until flow sorting. Gating against autofluorescence in the GFP channel was accomplished using Ldlr−/− lesional cells as a negative control. Cells were sorted on a FACS Aria 5 (non-sterile, 4° C.) directly into RNeasy Microkit lysis buffer (RLT with 1% β-mercaptoethanol). Samples were stored on ice until RNA isolation according to the manufacturer's protocol, after which RNA was stored at −80° C.

### Quantitative Real Time PCR

Total RNA was extracted from ground aortic arches as described (27) or from flow-sorted cells as described above. cDNA was prepared using Superscript III first-strand cDNA synthesis kits according to manufacturer's protocol. qRT-PCR was performed using Sybr Green (Life Technologies) according to manufacturer's recommendations and expression of target genes was normalized to individual sample GAPDH levels. Primers used to amplify p16^{Ink4a}, p19Arf, p21, Mmp3, Mmp13, Il1α, Tnfα and mRFP transcripts were previously described (15, 27).

### Histological Assessment of Plaques

Individual descending aorta plaques or intact brachiocephalic arteries were processed following a 12-h RT fixation in 4% PFA or 10% neutral buffered formaldehyde, respectively. All sections were 5 μm thick. For descending aorta plaques, at least 2 plaque-bearing sections obtained 250 μm apart were scored for all parameters. For brachiocephalic arteries, scoring was performed on sections collected in unbiased fashion 200 μm apart beginning at the brachiocephalic root and ending at the bifurcation into the right common carotid and subclavian arteries. Routine H&E staining was used in conjunction with Masson's trichrome (Sigma-Aldrich) or Voerhoff von Gieson (Polyscientific R&D) stains to measure fibrous cap thickness and broken elastic fibers, respectively. The fibrous cap was defined as an cosinophilic, Alcian blue-positive structure overlaying the plaque core, with no more than one macrophage foam cell overlying or interpenetrating the cap. Fifteen equally dispersed measurements of cap thickness were taken for each plaque section. The percentage of collagen was measured using blue-stained area in Masson's trichrome, with the plaque cross sectional area measured only above elastic fiber closest to the lumen.

### General Features of Senolytic Agents

Senolytic agents suitable for use in this invention include but are not limited to the compounds described in this section. Many senolytic agents share the characteristic that, at certain dosages, concentrations, or modes of delivery, the senolytic agents differentially or selectively kill or clear senescent cells in a mammal to which they are administered or in an in vitro assay. Exemplary senolytic agents are explained in the sections that follow.

### MDM2 Inhibitors

In certain embodiments, the senolytic agent may be an MDM2 inhibitor. An MDM2 (murine double minute 2) inhibitor that may be used in the methods for selectively killing senescent cells and treating or preventing (i.e., reducing or decreasing the likelihood of occurrence or development of) a senescence-associated disease or disorder may be a small molecule compound that belongs to any one of the following classes of compounds, for example, a cis-imidazoline compound, a spiro-oxindole compound, a benzodiazepine compound, a piperidinone compound, a tryptamine compound, and CGM097, and related analogs. In certain embodiments, the MDM2 inhibitor is also capable of binding to and inhibiting an activity of MDMX (murine double minute X, which is also known as HDMX in humans). The human homolog of MDM2 is called HDM2 (human double minute 2) in the art. Therefore, when a subject treated by the methods described herein is a human subject, the compounds described herein as MDM2 inhibitors also inhibit binding of HDM2 to one or more of its ligands.

MDM2 is described in the art as an E3 ubiquitin ligase that can promote tumor formation by targeting tumor suppressor proteins, such as p53, for proteasomal degradation through the 26S proteasome (see, e.g., Haupt et al. Nature 387: 296-299 1997; Honda et al., FEBS Lett 420: 25-27 (1997); Kubbutat et al., Nature 387: 299-303 (1997)). MDM2 also affects p53 by directly binding to the N-terminal end of p53, which inhibits the transcriptional activation function of p53 (see, e.g., Momand et al., Cell 69: 1237-1245 (1992); Oliner et al., Nature 362: 857-860 (1993)). Mdm2 is in turn regulated by p53; p53 response elements are located in the promoter of the Mdm2 gene (see, e.g., Barak et al., EMBO J 12:461-68 (1993)); Juven et al., Oncogene 8:3411-16 (1993)); Perry et al., Proc. Natl. Acad. Sci. 90:11623-27 (1993)). The existence of this negative feedback loop between p53 and Mdm2 has been confirmed by single-cell studies (see. e.g., Lahav, Exp. Med. Biol. 641:28-38 (2008)). See also Manfredi, Genes & Development 24:1580-89 (2010). Reports have described several activities and biological functions of MDM2.

These reported activities include the following: acts as a ubiquitin ligase E3 toward itself and ARRB1; permits nuclear export of p53; promotes proteasome-dependent ubiquitin-independent degradation of retinoblastoma RB 1 protein; inhibits DAXX-mediated apoptosis by inducing its ubiquitination and degradation; component of TRIM28/KAP1-MDM2-p53 complex involved in stabilizing p53; component of TRIM28/KAP1-ERBB4-MDM2 complex that links growth factor and DNA damage response pathways; mediates ubiquitination and subsequent proteasome degradation of DYRK2 in the nucleus; ubiquitinates IGF1R and SNAI1 and promotes them to proteasomal degradation. MDM2 has also been reported to induce mono-ubiquitination of the transcription factor FOXO4 (see, e.g., Brenkman et al., PLOS One 3(7):e2819, doi: 10.1371/journal.pone.0002819). The MDM2 inhibitors described herein may disrupt the interaction between MDM2 and any one or more of the aforementioned cellular components.

In one embodiment, a compound useful for the methods described herein is a cis-imidazoline small molecule inhibitor. Cis-imidazoline compounds include those called nutlins in the art. Similar to other MDM2 inhibitors described herein, nutlins are cis-imidazoline small molecule inhibitors of the interaction between MDM2 and p53 (see Vassilev et al., Science 303 (5659): 844-48 (2004)). Exemplary cis-imidazolines compounds that may be used in the methods for selectively killing senescent cells and treating or preventing (i.e., reducing or decreasing the likelihood of occurrence or development of) a senescence-associated disease or disorder are described in U.S. Pat. Nos. 6,734,302; 6,617,346; 7,705,007 and in U.S. Patent Application Publication Nos. 2005/0282803; 2007/0129416; 2013/0225603. In certain embodiments, the methods described herein comprise use of a nutlin compound called Nutlin-1; or a nutlin compound called Nutlin-2; or a Nutlin compound called Nutlin-3 (see CAS Registry No. 675576-98-4 and No. 548472-68-0). The active enantiomer of Nutlin-3 (4-[[4S,5R)-4,5-bis(4-chlorophenyl)-4,5-dihydro-2-[4-methoxy-2-(1-methylethoxy)phenyl]-1H-imidazol-1-yl]carbonyl]-2-piperazinone) is called Nutlin-3a in the art. In certain embodiments, the methods described herein comprise use of Nutlin-3a for selectively killing senescent cells.

Nutlin-3 is described in the art as a nongenotoxic activator of the p53 pathway, and the activation of p53 is controlled by the murine double minute 2 (MDM2) gene. The MDM2 protein is an E3 ubiquitin ligase and controls p53 half-life by way of ubiquitin-dependent degradation. Nutlin-3a has been investigated in pre-clinical studies (e.g., with respect to pediatric cancers) and clinical trials for treatment of certain cancers (e.g., retinoblastoma). To date in vitro and pre-clinical studies with Nutlin-3 have suggested that the compound has variable biological effects on the function of cells exposed to the compound. For example, Nutlin-3 reportedly increases the degree of apoptosis of cancer cells in hematological malignancies including B-cell malignancies (see. e.g., Zauli et al., Clin. Cancer Res. 17:762-70 (2011; online publication on Nov. 24, 2010) and references cited therein) and in combination with other chemotherapeutic drugs, such as dasatinib, the cytotoxic effect appears synergistic (see, e.g., Zauli et al., supra).

More generally, a family of MDM2 inhibitors that includes Nutlin-3 may be represented by Formula (I):

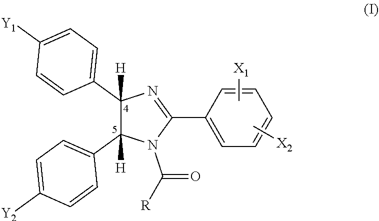

wherein R is selected from saturated and unsaturated 5- and 6-membered rings containing at least one hetero atom, wherein the hetero atom is selected from S. N and O and is optionally substituted with a group selected from lower alkyl, cycloalkyl, —C═O—R₁, hydroxy, lower alkyl substituted with hydroxy, lower alkyl substituted with lower alkoxy, lower alkyl substituted with —NH₂, lower alkyl substituted with —C═O—R₁, N-lower alkyl, —SO₂CH₃, ═O and —CH₂C═OCH₃;

R₁ is selected from hydrogen, lower alkyl, —NH₂, —N-lower alkyl, lower alkyl substituted with hydroxy, lower alkyl substituted with —NH₂, and a 5- or 6-membered saturated ring containing at least one hetero atom selected from S, N and O;

X₁ and X₂ are each independently selected from hydrogen, lower alkoxy, —CH₂OCH₃, —CH₂OCH₂CH₃, —OCH₂CF₃, and —OCH₂CH₂F; and

Y₁ and Y₂ are each independently selected from —Cl, —Br, —NO₂, —C≡N, and —C≡CH;

wherein the composition contains a formulation of the compound suitable for administration to subject who has atherosclerosis; and

wherein the formulation of the composition and the amount of the compound in the unit dose configure the unit dose to be effective in treating the atherosclerosis by eliminating p16 positive senescent cells in or around atherosclerotic plaques in the subject, thereby stabililzing the plaques so as to reduce the risk that the plaques will rupture.

Another exemplary cis-imidazoline small molecule compound useful for selectively killing senescent cells is RG-7112 (Roche) (CAS No: 939981-39-2; IUPAC name: ((4S,5R)-2-(4-(tert-butyl)-2-ethoxyphenyl)-4,5-bis(4-chlorophenyl)-4,5-dimethyl-4,5-dihydro-1H-imidazol-1-yl)(4-(3-(methylsulfonyl)propyl)piperazin-1-yl)methanone. See U.S. Pat. No. 7,851,626; Tovar et al., Cancer Res. 72:2587-97 (2013).

The MDM2 inhibitor may be a cis-imidazoline compound called RG7338 (Roche) (IUPAC Name: 4-((2R,3 S,4R,5 S)-3-(3-chloro-2-fluorophenyl)-4-(4-chloro-2-fluorophenyl)-4-cyano-5-neopentylpyrrolidine-2-carboxamido)-3-methoxybenzoic acid) (CAS 1229705-06-9); Ding et al., J. Med. Chem. 56(14):5979-83. Doi: 10.1021/jm400487c. Epub 2013 Jul. 16; Zhao et al., J. Med. Chem. 56(13):5553-61 (2013) doi: 10.1021/jm4005708. Epub 2013 Jun. 20). Yet another exemplary nutlin compound is RO5503781. Other potent cis-imidazoline small molecule compounds include dihydroimidazothiazole compounds (e.g., DS-3032b; Daiichi Sankyo) described by Miyazaki, (see, e.g., Miyazaki et al., Bioorg. Med. Chem. Lett. 23(3):728-32 (2013) doi: 10.1016/j.bmcl.2012.11.091. Epub 2012 Dec. 1; Miyazaki et al., Bioorg. Med. Chem. Lett. 22(20):6338-42 (2012) doi: 10.1016/j.bmcl.2012.08.086. Epub 2012 Aug. 30; Int'l Patent Appl. Publ. No. WO 2009/151069 (2009)).

Another cis-imidazoline compound that may be used in the methods described herein is a dihydroimidazothiazole compound. Alternatively, the MDM2 small molecule inhibitor is a spiro-oxindole compound. See, for example, compounds described in Ding et al., J. Am. Chem. Soc. 2005:127:10130-31; Shangary et al., Proc Natl Acad Sci USA 2008:105:3933-38; Shangary et al., Mol Cancer Ther 2008:7:1533-42; Shangary et al., Mol Cancer Ther 2008:7:1533-42; Hardcastle et al., Bioorg. Med. Chem. Lett. 15:1515-20 (2005); Hardcastle et al., J. Med. Chem. 49(21):6209-21 (2006), Watson et al., Bioorg. Med. Chem. Lett. 21(19):5916-9 (2011) doi: 10.1016/j.bmcl.2011.07.084. Epub 2011 Aug. 9. Other examples of spiro-oxindole compounds that are MDM2 inhibitors are called in the art MI-63, MI-126; MI-122, MI-142, MI-147, MI-18, MI-219, MI-220, MI-221, and MI-773. Another specific spiro-oxindole compound is 3-(4-chlorophenyl)-3-((1-(hydroxymethyl)cyclopropyl)methoxy)-2-(4-nitrobenzyl)isoindolin-1-one. Another compound is called M1888 (see, e.g., Zhao et al., J. Med. Chem. 56(13):5553-61 (2013); Int'l Patent Appl. Publ. No. WO 2012/065022).

The MDM2 small molecule inhibitor may be a benzodiazepinedione (see, e.g., Grasberger et al., J Med Chem 2005; 48:909-12; Parks et al., Bioorg Med Chem Lett 2005:15:765-70 Raboisson et al., Bioorg. Med. Chem. Lett. 15:1857-61 (2005); Koblish et al., Mol. Cancer Ther. 5:160-69 (2006)). Benzodiazepinedione compounds that may be used in the methods described herein include 1,4-benzodiazepin-2,5-dione compounds. Examples of benzodiazepinedione compounds include 5-[(3S)-3-(4-chlorophenyl)-4-[(R)-1-(4-chlorophenyl)ethyl]-2,5-dioxo-7-phenyl-1,4-diazepin-1-yl]valeric acid and 5-[(3S)-7-(2-bromophenyl)-3-(4-chlorophenyl)-4-[(R)-1-(4-chlorophenyl)ethyl]-2,5-dioxo-1,4-diazepin-1-yl]valeric acid (see, e.g., Raboisson et al., supra). Other benzodiazepinedione compounds are called in the art TDP521252 (IUPAC Name: 5-[(3S)-3-(4-chlorophenyl)-4-[(1R)-1-(4-chlorophenyl)ethyl]-7-ethynyl-2,5-dioxo-3H-1,4-benzodiazepin-1-yl]pentanoic acid) and TDP665759 (IUPAC Name: (3S)-4-[(R)-1-(2-amino-4-chlorophenyl)ethyl]-3-(4-chlorophenyl)-7-iodo-1-[3-(4-methylpiperazin-1-yl)propyl]-3H-1,4-benzodiazepine-2,5-dione) (see, e.g., Parks et al., supra; Koblish et al., supra) (Johnson & Johnson, New Brunswick, N.J.).

In yet another embodiment, the MDM2 small molecule inhibitor is a terphenyl (see, e.g., Yin et al., Angew Chem Int Ed Engl 2005; 44:2704-707; Chen et al., Mol Cancer Ther 2005:4:1019-25). In yet another specific embodiment, the MDM2 inhibitor that may be used in the methods described herein is a quilinol (see, e.g., Lu et al., J Med Chem 2006; 49:3759-62). In yet another certain embodiment, the MDM2 inhibitor is a chalcone (see, e.g., Stoll et al., Biochemistry 2001; 40:336-44). In yet another particular embodiment, the MDM2 inhibitor is a sulfonamide (e.g., NSC279287) (see, e.g., Galatin et al., J Med Chem 2004; 47:4163-65).

In other embodiments, a compound that may be used in the methods described herein is a tryptamine, such as serdemetan (JNJ-26854165; chemical name: N1-(2-(1H-indol-3-yl)ethyl)-N4-(pyridine-4-yl)benzene-1,4-diamine; CAS No. 881202-45-5) (Johnson & Johnson, New Brunswick, N.J.). Serdemetan is a tryptamine derivative that activates p53 and acts as a HDM2 ubiquitin ligase antagonist (see, e.g., Chargari et al., Cancer Lett. 312(2):209-18 (2011) doi: 10.1016/j.canlet.2011.08.011. Epub 2011 Aug. 22; Kojima et al., Mol. Cancer Ther. 9:2545-57 (2010); Yuan et al., J. Hematol. Oncol. 4:16 (2011)).

In other particular embodiments, MDM2 small molecule inhibitors that may be used in the methods described herein include those described in Rew et al., J. Med. Chem. 55:4936-54 (2012): Gonzalez-Lopez de Turiso et al., J. Med. Chem. 56:4053-70 (2013): Sun et al., J. Med. Chem. 57:1454-72 (2014); Gonzalez et al., J. Med. Chem. 2014 Mar. 4 [Epub ahead of print]; Gonzalez et al., J. Med. Chem. 2014 Mar. 6 [Epub ahead of print].

In still other embodiments, the MDM2 inhibitor is a piperidinone compound. An example of a potent MDM2 piperidinone inhibitor is AM-8553 ({(3R,5R,6S)-5-(3-Chlorophenyl)-6-(4-chlorophenyl)-1-[(2S,3S)-2-hydroxy-3-pentanyl]-3-methyl-2-oxo-3-piperidinyl}acetic acid; CAS No. 1352064-70-0) (Amgen. Thousand Oaks, Calif.).

In other particular embodiments, an MDM2 inhibitor that may be used in the methods described herein is a piperidine (Merck, Whitehouse Station, N.J.) (see, e.g., Int'l Patent Appl. Publ. No. WO 2011/046771). In other embodiments, an MDM2 inhibitor that may be used in the methods is an imidazole-indole compound (Novartis) (see. e.g., Int'l Patent Appl. Publ. No. WO 2008/119741).

Examples of compounds that bind to MDM2 and to MDMX and that may be used in the methods described herein include RO-2443 and RO-5963 ((Z)-2-(4-((6-Chloro-7-methyl-1H-indol-3-yl)methylene)-2,5-dioxoimidazolidin-1-yl)-2-(3,4-difluorophenyl)-N-(1,3-dihydroxypropan-2-yl)acetamide) (see, e.g., Graves et al., Proc. Natl. Acad. Sci. USA 109:11788-93 (2012); see also, e.g., Zhao et al., 2013, BioDiscovery, supra). In another specific embodiment, an MDM2 inhibitor referred to in the art as CGM097 may be used in the methods described herein for selectively killing senescent cells and for treating a senescence-associated disease or disorder.

### Inhibitors of Bcl-2 Anti-Apoptotic Family of Proteins

In certain embodiments, the senolytic agent may be an inhibitor of one or more proteins in the Bcl-2 family. In certain embodiments, the at least one senolytic agent is selected from an inhibitor of one or more Bcl-2 anti-apoptotic protein family members wherein the inhibitor inhibits at least Bcl-xL. Inhibitors of Bcl-2 anti-apoptotic family of proteins alter at least a cell survival pathway. Apoptosis activation may occur via an extrinsic pathway triggered by the activation of cell surface death receptors or an intrinsic pathway triggered by developmental cues and diverse intracellular stresses. This intrinsic pathway, also known as the stress pathway or mitochondrial pathway, is primarily regulated by the Bcl-2 family, a class of key regulators of caspase activation consisting of anti-apoptotic (pro-survival) proteins having BH1-BH4 domains (Bcl-2 (i.e., the Bcl-2 protein member of the Bcl-2 anti-apoptotic protein family), Bcl-xL, Bcl-w, A1, MCL-1, and Bcl-B); pro-apoptotic proteins having BH1, BH2, and BH3 domains (BAX, BAK, and BOK); and pro-apoptotic BH3-only proteins (BIK, BAD, BID, BIM, BMF, HRK, NOXA, and PUMA) (see, e.g., Cory et al., Nature Reviews Cancer 2:647-56 (2002): Cory et al., Cancer Cell 8:5-6 (2005); Adams et al., Oncogene 26:1324-1337 (2007)). Bcl-2 anti-apoptotic proteins block activation of pro-apoptotic multi-domain proteins BAX and BAK (see, e.g., Adams et al., Oncogene 26:1324-37 (2007)).

It is hypothesized that BH3-only proteins unleashed by intracellular stress signals bind to anti-apoptotic Bcl-2 like proteins via a BH3 “ligand” to a “receptor” BH3 binding groove formed by BH1-3 regions on anti-apoptotic proteins, thereby neutralizing the anti-apoptotic proteins (see, e.g., Letai et al., Cancer Cell 2:183-92 (2002); Adams et al., Oncogene, supra). BAX and BAK can then form oligomers in mitochondrial membranes, leading to membrane permeabilization, release of cytochrome C, caspase activation, and ultimately apoptosis (see, e.g., Adams et al., Oncogene, supra).

As used herein and unless otherwise stated, a Bcl-2 family member that is inhibited by the agents described herein is a pro-survival (anti-apoptotic) family member. The senolytic agents used in the methods described herein inhibit one or more functions of the Bcl-2 anti-apoptotic protein, Bcl-xL (which may also be written herein and in the art as Bcl-xL, Bcl-XL, Bcl-xl, or Bcl-XL). In certain embodiments, in addition to inhibiting Bcl-xL function, the inhibitor may also interact with and/or inhibit one or more functions of Bcl-2 (i.e., Bcl-xL/Bcl-2 inhibitors). In yet another certain embodiment, senolytic agents used in the methods described herein are classified as inhibitors of each of Bcl-xL and Bcl-w (i.e., Bcl-xL/Bcl-w inhibitors). In still another specific embodiment, senolytic agents used in the methods described herein that inhibit Bcl-xL may also interact with and inhibit one or more functions of each of Bcl-2 (i.e., the Bcl-2 protein) and Bcl-w (i.e., Bcl-xL/Bcl-2/Bcl-w inhibitors), thereby causing selective killing of senescent cells. In certain embodiments, a Bcl-2 anti-apoptotic protein inhibitor interferes with the interaction between the Bcl-2 anti-apoptotic protein family member (which includes at least Bcl-xL) and one or more ligands or receptors to which the Bcl-2 anti-apoptotic protein family member would bind in the absence of the inhibitor. In other particular embodiments, an inhibitor of one or more Bcl-2 anti-apoptotic protein family members wherein the inhibitor inhibits at least Bcl-xL specifically binds only to one or more of Bcl-xL, Bcl-2. Bcl-w and not to other Bcl-2 anti-apoptotic Bcl-2 family members, such as Mcl-1 and Bcl-2A1.

In still another embodiment, the senolytic agent used in the methods described herein is a Bcl-xL selective inhibitor and inhibits one or more functions of Bcl-xL. Such senolytic agents that are Bcl-xL selective inhibitors do not inhibit the function of one or more other Bcl-2 anti-apoptotic proteins in a biologically or statistically significant manner. Bcl-xL may also be called Bcl-2L1, Bcl-2-like 1, Bcl-X, Bcl-2L, Bcl-xL, or Bcl-X herein and in the art. In one embodiment, Bcl-xL selective inhibitors alter (e.g., reduce, inhibit, decrease, suppress) one or more functions of Bcl-xL but do not significantly inhibit one or more functions of other proteins in the Bcl-2 anti-apoptotic protein family (e.g., Bcl-2 or Bcl-w). In certain embodiments, a Bcl-xL selective inhibitor interferes with the interaction between Bcl-xL and one or more ligands or receptors to which Bcl-xL would bind in the absence of the inhibitor. In certain particular embodiments, a senolytic agent that inhibits one or more of the functions of Bcl-xL selectively binds to human Bcl-xL but not to other proteins in the Bcl-2 family, which effects selective killing of senescent cells.

Bcl-xL is an anti-apoptotic member of the Bcl-2 protein family. Bcl-xL also plays an important role in the crosstalk between autophagy and apoptosis (see, e.g., Zhou et al., FEBS J. 278:403-13 (2011)). Bcl-xL also appears to play a role in bioenergetic metabolism, including mitochondrial ATP production, Ca2+ fluxes, and protein acetylation, as well as on several other cellular and organismal processes such as mitosis, platelet aggregation, and synaptic efficiency (see, e.g., Michels et al., International Journal of Cell Biology, vol. 2013, Article ID 705294, 10 pages, 2013. doi: 10.1155/2013/705294). In certain embodiments, the Bcl-xL inhibitors described herein may disrupt the interaction between Bcl-xL and any one or more of the aforementioned BH3-only proteins to promote apoptosis in cells.

In certain embodiments, a Bcl-xL inhibitor is a selective inhibitor, meaning, that it preferentially binds to Bcl-xL over other anti-apoptotic Bcl-2 family members (e.g., Bcl-2, MCL-1, Bcl-w, Bcl-b, and BFL-1/A1).

Methods for measuring binding affinity of a Bcl-xL inhibitor for Bcl-2 family proteins are known in the art. By way of example, binding affinity of a Bcl-xL inhibitor may be determined using a competition fluorescence polarization assay in which a fluorescent BAK BH3 domain peptide is incubated with Bcl-xL protein (or other Bcl-2 family protein) in the presence or absence of increasing concentrations of the Bcl-XL inhibitor as previously described (see, e.g., U.S. Patent Publication 20140005190; Park et al., Cancer Res. 73:5485-96 (2013); Wang et al., Proc. Natl. Acad. Sci USA 97:7124-9 (2000); Zhang et al., Anal. Biochem. 307:70-5 (2002); Bruncko et al., J. Med. Chem. 50:641-62 (2007)). Percent inhibition may be determined by the equation: 1-[(mP value of well−negative control)/range)]×100%. Inhibitory constant (Ki) value is determined by the formula: Ki=[I]50/([L]50/Kd+[P]0/Kd+1) as described in Bruncko et al., J. Med. Chem. 50:641-62 (2007) (see, also, Wang, FEBS Lett. 360:111-114 (1995)).

Agents (e.g., Bcl-xL selective inhibitors, Bcl-xL/Bcl-2 inhibitors, Bcl-xL/Bcl-2/Bcl-w inhibitors, Bcl-xL/Bcl-w inhibitors) used in the methods described herein that selectively kill senescent cells include, by way of example, a small molecule.

In particular embodiments, the Bcl-xL inhibitor is a small molecule compound that belongs to any one of the following classes of compounds, for example, a benzothiazole-hydrazone compound, aminopyridine compound, benzimidazole compound, tetrahydroquinoline compound, and phenoxyl compound and related analogs.

In one embodiment, a Bcl-xL selective inhibitor useful for the methods described herein is a benzothiazole-hydrazone small molecule inhibitor. Benzothiazole-hydrazone compounds include WEHI-539 (5-[3-[4-(aminomethyl)phenoxy]propyl]-2-[(8E)-8-(1,3-benzothiazol-2-ylhydrazinylidene)-6,7-dihydro-5H-naphthalen-2-yl]-1,3-thiazole-4-carboxylic acid), a BH3 peptide mimetic that selectively targets Bcl-xL (see, e.g., Lessene et al., Nature Chemical Biology 9:390-397 (2013)). In certain embodiments, the methods described herein comprise use of WEHI-539 for selectively killing senescent cells.

In other embodiments, the Bcl-xL selective inhibitor is an aminopyridine compound. An aminopyridine compound that may be used as a selective Bcl-xL inhibitor is BXI-61 (3-[(9-amino-7-ethoxyacridin-3-yl)diazenyl]pyridine-2,6-diamine) (see, e.g., Park et al., Cancer Res. 73:5485-96 (2013); U.S. Patent Publ. No. 2009-0118135). In certain embodiments, the methods described herein comprise use of BXI-61 for selectively killing senescent cells.

In still other embodiments, the Bcl-xL selective inhibitor that may be used in the methods described herein is a benzimidazole compound. An example of a benzimidazole compound that may be used as a selective Bcl-XL inhibitor is BXI-72 (2′-(4-Hydroxyphenyl)-5-(4-methyl-1-piperazinyl)-2,5′-bi(1H-benzimidazole) trihydrochloride) (see, e.g., Park et al., supra). In certain embodiments, the methods described herein comprise use of BXI-72 for selectively killing senescent cells.

In yet another embodiment, the Bcl-xL selective inhibitor is a tetrahydroquinoline compound (see, e.g., U.S. Patent Publ. No. 2014-0005190). Examples of tetrahydroquinoline compounds that may be used as selective Bcl-xL inhibitors are shown in Table 1 of U.S. Patent Publ. No. 2014-0005190 and described therein. Other inhibitors described therein may inhibit other Bcl-2 family members (e.g., Bcl-2) in addition to Bcl-xL.

In other embodiments, a Bcl-xL selective inhibitor is a phenoxyl compound. An example of a phenoxyl compound that may be used as a selective Bcl-xL inhibitor is 2[[3-(2,3-dichlorophenoxy) propyl]amino]ethanol (2,3-DCPE) (see, Wu et al., Cancer Res. 64:1110-1113 (2004)). In certain embodiments, the methods described herein comprise use of 2,3-DCPE for selectively killing senescent cells.

In still another embodiment, an inhibitor of a Bcl-2 anti-apoptotic family member that inhibits at least Bcl-xL is described in U.S. Pat. No. 8,232,273. In a particular embodiment, the inhibitor is a Bcl-xL selective inhibitor called A-1155463 (see, e.g., Tao et al., ACS Med. Chem. Lett., 2014, 5(10): 1088-1093).

In other embodiments, a senolytic agent of interest inhibits other Bcl-2 anti-apoptotic family members in addition to Bcl-xL. For example, methods described herein comprise use of Bcl-xL/Bcl-2 inhibitors, Bcl-xL/Bcl-2/Bcl-w inhibitors, and Bcl-xL/Bcl-w inhibitors and analogs thereof. In certain embodiments, the inhibitors include compounds that inhibit Bcl-2 and Bcl-xL, which inhibitors may also inhibit Bcl-w. Examples of these inhibitors include ABT-263 (4-[4-[[2-(4-chlorophenyl)-5,5-dimethylcyclohexen-1-yl]methyl]piperazin-1-yl]-N-4-[[(2R)-4-morpholin-4-yl-1-phenylsulfanylbutan-2-yl]amino]-3-(trifluoromethylsulfonyl)phenyl]sulfonylbenzamide or IUPAC, (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide) (see, e.g., Park et al., 2008, J. Med. Chem. 51:6902; Tse et al., Cancer Res., 2008, 68:3421; Int'l Patent Appl. Pub. No. WO 2009/155386; U.S. Pat. Nos. 7,390,799, 7,709,467, 7,906,505, 8,624,027) and ABT-737 (4-[4-[(4′-Chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]benzamide, Benzamide, 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]- or 4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (see, e.g., Oltersdorf et al., Nature, 2005, 435:677; U.S. Pat. No. 7,973,161; U.S. Pat. No. 7,642,260). In other embodiments, the Bcl-2 anti-apoptotic protein inhibitor is a quinazoline sulfonamide compound (see, e.g., Sleebs et al., 2011. J. Med. Chem. 54:1914). In still another embodiment, the Bcl-2 anti-apoptotic protein inhibitor is a small molecule compound as described in Zhou et al., J. Med. Chem., 2012, 55:4664 (see, e.g., Compound 21 (R)-4-(4-chlorophenyl)-3-(3-(4-(4-(4-((4-(dimethylamino)-1-(phenylthio)butan-2-yl)amino)-3-nitrophenylsulfonamido)phenyl)piperazin-1-yl)phenyl)-5-ethyl-1-methyl-1H-pyrrole-2-carboxylic acid) and Zhou et al., J. Med. Chem. 2012, 55:6149 (see, e.g., Compound 14 (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid; Compound 15 (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-isopropyl-2-methyl-1H-pyrrole-3-carboxylic acid). In other embodiments, the Bcl-2 anti-apoptotic protein inhibitor is a Bcl-2/Bcl-xL inhibitor such as BM-1074 (see, e.g., Aguilar et al., 2013. J. Med. Chem. 56:3048); BM-957 (see, e.g., Chen et al., 2012, J. Med. Chem. 55:8502): BM-1197 (see, e.g., Bai et al., PLoS One 2014 Jun. 5:9(6):e99404. Doi: 10.1371/journal.pone. 009904); U.S. Patent Appl. No. 2014/0199234; N-acylsufonamide compounds (see, e.g., Int'l Patent Appl. Pub. No. WO 2002/024636, Int'l Patent Appl. Pub. No. WO 2005/049593. Int'l Patent Appl. Pub. No. WO 2005/049594, U.S. Pat. No. 7,767,684, U.S. Pat. No. 7,906,505). In still another embodiment, the Bcl-2 anti-apoptotic protein inhibitor is a small molecule macrocyclic compound (see, e.g., Int'l Patent Appl. Pub. No. WO 2006/127364, U.S. Pat. No. 7,777,076). In yet another embodiment, the Bcl-2 anti-apoptotic protein inhibitor is an isoxazolidine compound (see, e.g., Int'l Patent Appl. Pub. No. WO 2008/060569, U.S. Pat. No. 7,851,637, U.S. Pat. No. 7,842,815).

In certain embodiments, the senolytic agent is a compound that is an inhibitor of Bcl-2, Bcl-w, and Bcl-xL, such as ABT-263 (Navitoclax) or ABT-737. In certain specific embodiments, the senolytic agent is a compound or a pharmaceutically acceptable salt, stereoisomer, tautomer, or prodrug thereof as illustrated below.

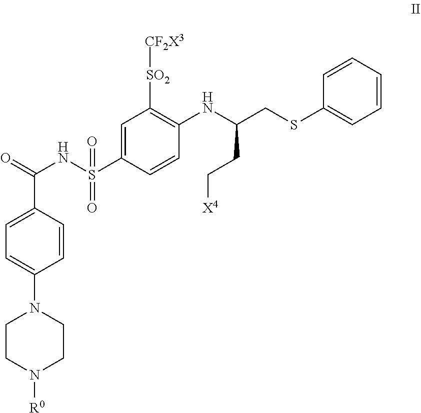

wherein X³ is Cl or F;

X⁴ is azepan-1-yl, morpholin-4-yl, 1,4-oxazepan-4-yl, pyrrolidin-1-yl, N(CH₃)₂, N(CH₃)(CH(CH₃)₂), 7-azabicyclo[2.2.1]heptan-1-yl or 2-oxa-5-azabicyclo[2.2.1]hept-5-yl, and R⁰ is

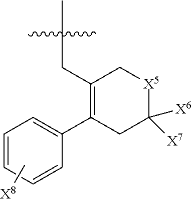

wherein X⁵ is CH₂, C(CH₃)₂, or CH₂CH₂; X⁶ and X⁷ are both hydrogen or are both methyl; and X⁸ is F, Cl, Br or I; or

X⁴ is azepan-1-yl, morpholin-4-yl, pyrrolidin-1-yl, N(CH₃)(CH(CH₃)₂) or 7-azabicyclo[2.2.1]heptan-1-yl, and R⁰ is

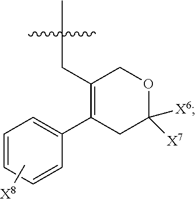

or

X⁴ is N(CH₃)₂ or morpholin-4-yl, and R⁰ is

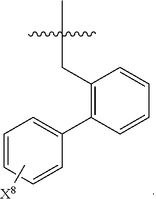

### Akt Kinase Inhibitors

In certain embodiments the senolytic agent is an Akt Kinase inhibitor. For example, a senolytic agent can be a small molecule compound and analogs thereof that inhibits Akt. In some embodiments, the senolytic agent is a compound that selectively inhibits Akt1, Akt2, and Akt3, relative to other protein kinases.

Akt inhibitors (which may also be called Akt kinase inhibitors or AKT kinase inhibitors) can be divided into six major classes based on their mechanisms of action (see, e.g., Bhutani et al., Infectious Agents and Cancer 2013, 8:49 doi: 10.1186/1750-9378-8-49). Akt is also called protein kinase B (PKB) in the art. The first class contains ATP competitive inhibitors of Akt and includes compounds such as CCT128930 and GDC-0068, which inhibit Akt2 and Akt1. This category also includes the pan-Akt kinase inhibitors such as GSK2110183 (afuresertib), GSK690693, and AT7867. The second class contains lipid-based Akt inhibitors that act by inhibiting the generation of PIP3 by PI3K. This mechanism is employed by phosphatidylinositol analogs such as Calbiochem Akt Inhibitors I, II and III or other PI3K inhibitors such as PX-866. This category also includes compounds such as Perifosine (KRX-0401) (Aetema Zentaris/Keryx). The third class contains a group of compounds called pseudosubstrate inhibitors. These include compounds such as AKTide-2 T and FOXO3 hybrid. The fourth class consists of allosteric inhibitors of AKT kinase domain, and include compounds such as MK-2206 (8-[4-(1-aminocyclobutyl)phenyl]-9-phenyl-2H-[1,2,4]triazolo[3,4-f][1,6]naphthyridin-3-one:dihydrochloride) (Merck & Co.) (see, e.g., U.S. Pat. No. 7,576,209). The fifth class consists of antibodies and include molecules such as GST-anti-Akt1-MTS. The last class comprises compounds that interact with the PH domain of Akt, and includes Triciribine and PX-316. Other compounds described in the art that act as AKT inhibitors include, for example, GSK-2141795 (GlaxoSmithKline), VQD-002, miltefosine, AZD5363, GDC-0068, and API-1.

The senolytic agent is a compound may be an Akt kinase inhibitor, which has the structure as shown below (also called MK-2206 herein and in the art), 8-[4-(1-aminocyclobutyl)phenyl]-9-phenyl-2H-[1,2,4]triazolo[3,4-f][1,6]naphthyridin-3-one) or a pharmaceutically acceptable salt, stereoisomer, tautomer, or prodrug thereof

### Drug Combinations

Since treating atherosclerotic plaques with senolytic agents does not prevent plaque regression, this invention provides therapeutic combinations whereby plaques can be stabilized and caused to regress at the same time. Regimens for causing rejection include prescription of a low fat or calorie reduced diet, exercise, and consumption of drugs that reduce circulating lipid levels. These include statins, exemplified by atorvastatin, cerivastatin, fluvastatin, lovastatin, mevastatin, pitavastatin, pravastatin, rosuvastatin, and simvastatin. A senolytic agent can be beneficially administered in combination with any of these.

Thus, the invention includes a method of improving the therapeutic effect of a regimen that is prescribed to a subject to promote regression of atherosclerotic plaques in their arterial vasculature. Such methods comprise administering to a subject in need thereof a senolytic agent as described in this disclosure, in an amount that is effective to inhibit or reverse thinning of fibrous caps on the atherosclerotic plaques, thereby inhibiting rupture of the plaques, without preventing the regimen from promoting regression of the plaques.

This invention also includes senolytic agents and lipid lowering drugs as a drug combination. The drugs can be formulated for administration together (such as a combined tablet). Alternatively, they can be separately formulated but sold together in the same package. Alternatively, they can be sold separately with information about how to combine them for an improved therapeutic effect.

### Definitions

A “senescent cell” is generally thought to be derived from a cell type that typically replicates, but as a result of aging or other event that causes a change in cell state, can no longer replicate. It remains metabolically active and commonly adopts a senescence associated secretory phenotype (SASP) that includes chemokines, cytokines and extracellular matrix and fibrosis modifying proteins and enzymes. The nucleus of senescent cells is often characterized by senescence-associated heterochromatin foci and DNA segments with chromatin alterations reinforcing senescence. Without implying any limitation on the practice of what is claimed in this disclosure that is not explicitly stated or required, the invention is premised on the hypothesis that senescent cells cause or mediate certain conditions associated with tissue damage or aging. For the purpose of practicing aspects of this invention, senescent cells can be identified as expressing at least one marker selected from p16, senescence-associated j-galactosidase, and lipofuscin; sometimes two or more of these markers, and other markers of SASP such as but not limited to interleukin 6, and inflammatory, angiogenic and extracellular matrix modifying proteins.

A “senescence associated” disease, disorder, or condition is a physiological condition that presents with one or more symptoms or signs, wherein a subject having the condition needs or would benefit from a lessening of such symptoms or signs. The condition is senescence associated if it is caused or mediated in part by senescent cells, which may be induced by multiple etiologic factors including age, DNA damage, oxidative stress, genetic defects, etc. Lists of senescence associated disorders that ca potentially be treated or managed using the methods and products taught in this disclosure include those discussed in this disclosure and the previous disclosures to which this application claims priority.

A compound is typically referred to as “senolytic” if it eliminates senescent cells, compared with replicative cells of the same tissue type, or quiescent cells lacking SASP markers. Alternatively, or in addition, a compound or combination may effectively be used according to this invention if it decreases the release of pathological soluble factors or mediators as part of the senescence associated secretory phenotype that play a role in the initial presentation or ongoing pathology of a condition, or inhibit its resolution. In this respect, the term “senolytic” is exemplary, with the understanding that compounds that work primarily by inhibiting rather than eliminating senescent cells (senescent cell inhibitors) can be used in a similar fashion with ensuing benefits.

“Small molecule” senolytic agents according to this invention have molecular weights less than 20,000 daltons, and are often less than 10,000, 5,000, or 2,000 daltons. Small molecule inhibitors are not antibody molecules or oligonucleotides, and typically have no more than five hydrogen bond donors (the total number of nitrogen-hydrogen and oxygen-hydrogen bonds), and no more than 10 hydrogen bond acceptors (all nitrogen or oxygen atoms).

Successful “treatment” of a liver disease according to this invention may have any effect that is beneficial to the subject being treated. This includes decreasing severity, duration, or progression of a condition, or of any adverse signs or symptoms resulting therefrom. In some circumstances, senolytic agents can also be used to prevent or inhibit presentation of a condition for which a subject is susceptible, for example, because of an inherited susceptibility of because of medical history.

A “therapeutically effective amount” is an amount of a compound of the present disclosure that (i) treats the particular disease, condition, or disorder, (ii) attenuates, ameliorates, or eliminates one or more symptoms of the particular disease, condition, or disorder, (iii) prevents or delays the onset of one or more symptoms of the particular disease, condition, or disorder described herein. (iv) prevents or delays progression of the particular disease, condition or disorder, or (v) at least partially reverses damage caused by the condition prior to treatment.

A “phosphorylated” form of a compound is a compound in which one or more —OH or —COOH groups have been substituted with a phosphate group which is either —OPO₃H₂ or —CₙPO₃H₂ (where n is 1 to 4), such that the phosphate group may be removed in vivo (for example, by enzymolysis). A non-phosphorylated or dephosphorylated form has no such group.

This invention includes senolytic agents that are adapted to “home” preferentially either to target hepatocytes, to cholangiocytes, or to both. Alternatively or in addition, senolytic agents can be adapted to home preferentially to senescent cells in the target tissue, characterized by expression of p16 or other senescent cell markers. Specific “homing” is a process by which an agent contacts a target cell, it is at least 5-times (preferably at least 20- or at least 100-times more likely to bind to the surface an/or be taken up into the target cell than to cells outside the liver. A “homing agent” or means for homing is a chemical moiety conjugated to a senolytic agent such that the chemical moiety causes preferential uptake of the senolytic agent by the target cell, resulting in selective elimination of the target cell.

Unless otherwise stated or required, all the compound structures referred to in the invention include conjugate acids and bases having the same structure, crystalline and amorphous forms of those compounds, pharmaceutically acceptable salts, and dissolved and solid forms thereof, including, for example, polymorphs, solvates, hydrates, unsolvated polymorphs (including anhydrates), conformational polymorphs, and amorphous forms of the compounds, as well as mixtures thereof.

Except where otherwise stated or required, other terms used in the specification have their ordinary meaning.

### REFERENCES

1. Z. Cui, M. C. Willingham, The effect of aging on cellular immunity against cancer in SR/CR mice. Cancer immunology immunotherapy: CII 53, 473-478 (2004).
2. I. Tabas, G. Garcia-Cardena. G. K. Owens, Recent insights into the cellular biology of atherosclerosis. J Cell Biol 209, 13-22 (2015).
3. C. Weber, H. Noels, Atherosclerosis: current pathogenesis and therapeutic options. Nat Med 17, 1410-1422 (2011).
4. C. E. Myers, N. N. Mirza, J. Lustgarten, Immunity, cancer and aging: lessons from mouse models. Aging and disease 2, 512-523 (2011).
5. K. Sakakura et al., Pathophysiology of atherosclerosis plaque progression. Heart, lung & circulation 22, 399-411 (2013).
6. I Gorenne, M. Kavurma, S. Scott. M. Bennett, Vascular smooth muscle cell senescence in atherosclerosis. Cardiovascular research 72, 9-17 (2006).
7. T. Minamino et al., Endothelial cell senescence in human atherosclerosis: role of telomere in endothelial dysfunction. Circulation 105, 1541-1544 (2002).
8. J. C. Wang, M. Bennett, Aging and atherosclerosis: mechanisms, functional consequences, and potential therapeutics for cellular senescence. Circ Res 111, 245-259 (2012).
9. D. Munoz-Espin, M. Serrano, Cellular senescence: from physiology to pathology. Nat Rev Mol Cell Biol 15, 482-496 (2014).
10. J. Wang et al., Vascular Smooth Muscle Cell Senescence Promotes Atherosclerosis and Features of Plaque Vulnerability. Circulation 132, 1909-1919 (2015).
11. A. K. Khanna, Enhanced susceptibility of cyclin kinase inhibitor p21 knockout mice to high fat diet induced atherosclerosis. J Biomed Sci 16, 66 (2009).
12. J. Mercer, N. Figg. V. Stoneman, D. Braganza, M. R. Bennett, Endogenous p53 protects vascular smooth muscle cells from apoptosis and reduces atherosclerosis in ApoE knockout mice. Circ Res 96, 667-674 (2005).
13. H. Gonzalez-Navarro et al., p19(ARF) deficiency reduces macrophage and vascular smooth muscle cell apoptosis and aggravates atherosclerosis. J Am Coll Cardiol 55, 2258-2268 (2010).
14. K. Wouters et al., Bone marrow p16^{Ink4a}-deficiency does not modulate obesity, glucose homeostasis or atherosclerosis development. PLoS One 7, e32440 (2012).
15. C. L. Kuo et al., Cdkn2a is an atherosclerosis modifier locus that regulates monocyte/macrophage proliferation. Arterioscler Thromb Vasc Biol 31, 2483-2492 (2011).
16. M. Demaria et al., An Essential Role for Senescent Cells in Optimal Wound Healing through Secretion of PDGF-AA. Dev Cell 31, 722-733 (2014).
17. H. Williams, J. L. Johnson, K. G. Carson, C. L. Jackson, Characteristics of intact and ruptured atherosclerotic plaques in brachiocephalic arteries of apolipoprotein E knockout mice. Arterioscler Thromb Vasc Biol 22, 788-792 (2002).
18. D. J. Baker et al., Clearance of p16^{Ink4a}-positive senescent cells delays ageing-associated disorders. Nature 479, 232-236 (2011).
19. D. J. Baker et al., Naturally occurring p16(Ink4a)-positive cells shorten healthy lifespan. Nature 530, 184-189 (2016).
20. J. Chang et al., Clearance of senescent cells by ABT263 rejuvenates aged hematopoietic stem cells in mice. Nat Med 22, 78-83 (2016).
21. Y. Nakashima, E. W. Raines, A. S. Plump, J. L. Breslow, R. Ross, Upregulation of VCAM-1 and ICAM-1 at atherosclerosis-prone sites on the endothelium in the ApoE-deficient mouse. Arterioscler Thromb Vasc Biol 18, 842-851 (1998).
22. J. L. Johnson, C. L. Jackson, Atherosclerotic plaque rupture in the apolipoprotein E knockout mouse. Atherosclerosis 154, 399-406 (2001).
23. M. C. Clarke et al., Apoptosis of vascular smooth muscle cells induces features of plaque vulnerability in atherosclerosis. Nat Med 12, 1075-1080 (2006).
24. N. Maldonado et al., A mechanistic analysis of the role of microcalcifications in atherosclerotic plaque stability: potential implications for plaque rupture. American journal of physiology. Heart and circulatory physiology 303, H619-628 (2012).
25. C. Silvestre-Roig et al., Atherosclerotic plaque destabilization: mechanisms, models, and therapeutic strategies. Circ Res 114, 214-226 (2014).
26. S. M. Ghaderian, R. Akbarzadeh Najar, A. S. Tabatabaei Panah, Genetic polymorphisms and plasma levels of matrix metalloproteinases and their relationships with developing acute myocardial infarction. Coronary artery disease 21, 330-335 (2010).
27. A. V. Finn, M. Nakano, J. Narula, F. D. Kolodgie, R. Virmani, Concept of vulnerable/unstable plaque. Arterioscler Thromb Vasc Biol 30, 1282-1292 (2010).
28. K. Inoue et al., Serial coronary CT angiography-verified changes in plaque characteristics as an end point: evaluation of effect of statin intervention. JACC. Cardiovascular imaging 3, 691-698 (2010).
29. D. J. Baker et al., Naturally occurring p16-positive cells shorten healthy lifespan. Nature, (2016).
30. S. Curado, D. Y. Stainier. R. M. Anderson, Nitroreductase-mediated cell/tissue ablation in zebrafish: a spatially and temporally controlled ablation method with applications in developmental and regeneration studies. Nature protocols 3, 948-,954 (2008).
31. D. J. Baker et al., BubR1 insufficiency causes early onset of aging-associated phenotypes and infertility in mice. Nat Genet 36, 744-749 (2004).
32. C. A. Conover et al., Transgenic overexpression of pregnancy-associated plasma protein-A in murine arterial smooth muscle accelerates atherosclerotic lesion development. American journal of physiology. Heart and circulatory physiology 299, H284-291 (2010).

The several hypotheses presented in this disclosure provide a premise by way of which the reader may understand the invention. This premise is provided for the enrichment and appreciation of the reader. Practice of the invention does not require detailed understanding or application of the hypothesis. Except where stated otherwise, features of the hypothesis presented in this disclosure do not limit application or practice of the claimed invention. For example, except where the elimination of senescent cells expressing p16 or otherwise defined is explicitly required, the compounds and methodology of this invention may be used for treating the conditions described regardless of their effect on senescent cells. The invention may be practiced on patients of any age having the condition indicated, unless otherwise explicitly indicated or required.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed.

The invention claimed is:

1. A method of preventing progression of atherosclerosis in a subject, comprising contacting an atherosclerotic plaque in the subject with a compound that is either a means for selectively inhibiting Bcl 2 or Bcl-xL or a means for inhibiting mouse double minute 2 homolog (MDM2), wherein the compound is administered in an amount that is effective in eliminating p16 positive senescent cells from the plaque.

2. The method of claim 1, wherein the compound is a means for selectively inhibiting Bcl-2 or Bcl-xL.

3. The method of claim 2, wherein the compound is ABT-263 (Navitoclax), or a pharmaceutically acceptable salt thereof.

4. The method of claim 2, wherein the compound is selected from WEHI-539, A-1155463, ABT-737, ATB-199, Obatoclax, BXI-61, BXI-72, 2,3-DCPE, “Compound 21” (R)-4-(4-chlorophenyl)-3-(3-(4-(4-(4-((4-(dimethylamino)-1-(phenylthio)butan-2-yl)amino)-3-nitrophenylsulfonamido)phenyl)piperazin-1-yl)phenyl)-5-ethyl-1-methyl-1H-pyrrole-2-carboxylic acid, “Compound 14” (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid, “Compound 15” (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-isopropyl-2-methyl-1H-pyrrole-3-carboxylic acid, BM-957, BM-1074, BM-1197, and pharmaceutically acceptable salts thereof.

5. The method of claim 2, wherein the compound has the following structure: 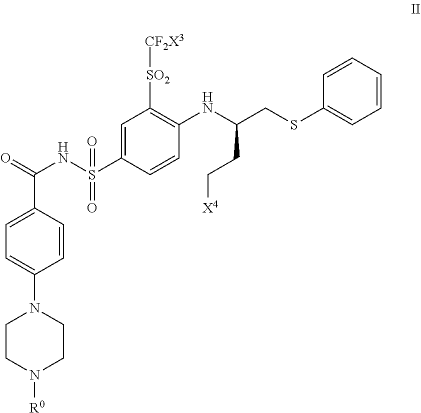
wherein X³ is Cl or F:
X⁴ is azepan-1-yl, morpholin-4-yl, 1,4-oxazepan-4-yl, pyrrolidin-1-yl, N(CH₃)₂, N(CH₃)(CH(CH₃)₂), 7-azabicyclo[2.2.1]heptan-1-yl or 2-oxa-5-azabicyclo[2.2.1]hept-5-yl, and R⁰ is 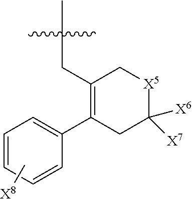
wherein X⁵ is CH₂, C(CH₃)₂, or CH₂CH₂; X⁶ and X⁷ are both hydrogen or are both methyl; and X⁸ is F, Cl, Br or I; or
X⁴ is azepan-1-yl, morpholin-4-yl, pyrrolidin-1-yl, N(CH₃)(CH(CH₃)₂) or 7-azabicyclo[2.2.1]heptan-1-yl, and R⁰ is 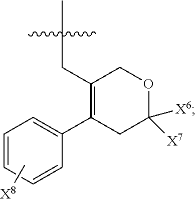
or
X⁴ is N(CH₃)₂ or morpholin-4-yl, and R⁰ is

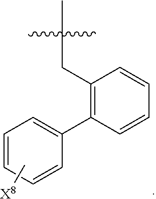

6. The method of claim 1, wherein the senescent cells are senescent foam cell macrophages.

7. The method of claim 1, wherein the senescent cells are positive for p16.

8. The method of claim 1, whereby thinning of fibrous caps on atherosclerotic plaques in the subject is inhibited or reversed

9. The method of claim 1, whereby the atherosclerotic plaque is stabilized, thereby reducing risk that the plaque will rupture.

10. The method of claim 1, wherein the compound is administered systemically.

11. The method of claim 1, wherein the compound is administered as a single dose, followed by a non-treatment interval of at least two weeks.

12. A method of improving the therapeutic effect of a regimen that is prescribed to a subject to promote regression of atherosclerotic plaques in their arterial vasculature,
the method comprising administering to the subject in combination with the regimen a compound that is either a means for selectively inhibiting Bcl 2 or Bcl-xL, or a means for inhibiting mouse double minute 2 homolog (MDM2),
wherein the compound is administered in an amount that is effective to inhibit or reverse thinning of fibrous caps on the atherosclerotic plaques, thereby inhibiting rupture of the plaques, without preventing the regimen from promoting regression of the plaques.

13. The method of claim 12, wherein the regimen includes consumption of a low fat or a calorie reduced diet.

14. The method of claim 12, wherein the regimen includes administration of a statin to the subject.

15. The method of claim 12, wherein the compound is a means for selectively inhibiting Bcl-2 or Bcl-xL.

16. The method of claim 15, wherein the compound is ABT-263 (Navitoclax), or a pharmaceutically acceptable salt thereof.

17. The method of claim 15, wherein the compound is selected from WEHI-539, A-1155463, ABT-737, ATB-199, Obatoclax, BXI-61, BXI-72, 2,3-DCPE, “Compound 21” (R)-4-(4-chlorophenyl)-3-(3-(4-(4-(4-((4-(dimethylamino)-1-(phenylthio)butan-2-yl)amino)-3-nitrophenylsulfonamido)phenyl)piperazin-1-yl)phenyl)-5-ethyl-1-methyl-1H-pyrrole-2-carboxylic acid, “Compound 14” (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-ethyl-2-methyl-1H-pyrrole-3-carboxylic acid, “Compound 15” (R)-5-(4-Chlorophenyl)-4-(3-(4-(4-(4-((4-(dimethylamino)-1-(phenylthio)butan-2-yl)amino)-3-nitrophenylsulfonamido)phenyl)piperazin-1-yl)phenyl)-1-isopropyl-2-methyl-1H-pyrrole-3-carboxylic acid, BM-957, BM-1074, BM-1197, and pharmaceutically acceptable salts thereof.

18. The method of claim 15, wherein the compound has the following structure: 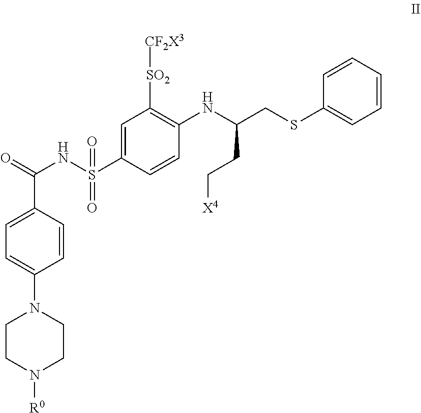
wherein X³ is Cl or F;
X⁴ is azepan-1-yl, morpholin-4-yl, 1,4-oxazepan-4-yl, pyrrolidin-1-yl, N(CH₃)₂, N(CH₃)(CH(CH₃)₂, 7-azabicyclo[2.2.1]heptan-1-yl or 2-oxa-5-azabicyclo[2.2.1]hept-5-yl, and R⁰ is 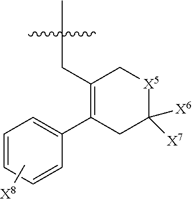
wherein X⁵ is CH₂, C(CH₃)₂, or CH₂CH₂; X⁶ and X⁷ are both hydrogen or are both methyl; and X⁸ is F, Cl, Br or I; or
X⁴ is azepan-1-yl, morpholin-4-yl, pyrrolidin-1-yl, N(CH₃)(CH(CH₃)₂) or 7-azabicyclo[2.2.1]heptan-1-yl, and R⁰ is 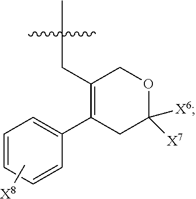
or
X⁴ is N(CH₃)₂ or morpholin-4-yl, and R⁰ is

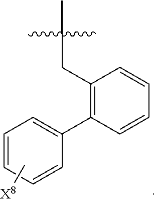

19. A unit dose of a pharmaceutical composition formulated for treatment of atherosclerosis, the composition containing an effective amount of a senolytic compound;
wherein the senolytic compound is a means for inhibiting Bcl-2, a means for inhibiting Bcl-xL, or a means for inhibiting MDM2, as hereintofore described or claimed;
wherein the formulation of the composition and the amount of the compound in the unit dose configure the unit dose to be effective in treating the atherosclerosis by eliminating p16 positive senescent cells in or around atherosclerotic plaques in the subject, thereby stabilizing the plaques so as to reduce the risk that the plaques will rupture.

20. A pharmaceutical product or product combination configured for treatment of atherosclerosis, comprising a unit dose according to claim 19 in combination with a statin.
